(19) United States
(12) Patent Application Publication
(10) Pub. No.: US 2002/0192665 A1
(43) Pub. Date: Dec. 19, 2002

(54) Compositions and methods for the therapeutic use of an atonal-associated sequence for a gastrointestinal condition

(76) Inventors: Huda Y. Zoghbi, Houston, TX (US); Qi Yang, The Woodlands, TX (US)

Correspondence Address: FULBRIGHT & JAWORSKI, LLP, 1301 MCKINNEY, SUITE 5100, HOUSTON, TX, 77010-3095, US

(21) Appl. No.: 10/004,717

(22) Filed: Dec. 5, 2001

Related U.S. Application Data
(63) Continuation of application No. 09/585,645, filed on Jun. 1, 2000.
(60) Provisional application No. 60/137,060, filed on Jun. 1, 1999.
(60) Provisional application No. 60/176,993, filed on Jan. 19, 2000.

(51) Int. Cl. C12Q 1/68; G01N 33/567; C12N 5/08

(52) U.S. Cl. 435/6; 435/366; 435/7.21

(57) ABSTRACT

Compositions and methods are disclosed for the therapeutic use of an atonal-associated nucleic acid or amino acid sequence. Also, an animal heterozygous for an atonal-associated gene inactivation is also disclosed having at least one atonal-associated nucleic acid sequence replaced by insertion of a heterologous nucleic acid sequence used to detect expression driven by an atonal-associated promoter sequence, wherein the inactivation of the atonal-associated nucleic acid sequence prevents expression of the atonal-associated gene.

This application claims priority to a provisional application Serial No. 60/137,060 filed Jun. 1, 1999; a second provisional application Serial No. 60/176,993 filed Jan. 19, 2000; and a nonprovisional application Ser. No. 09/585,645, filed Jun. 1, 2000.

The work herein was supported by grants from the United States Government. The United States Government may have certain rights in the invention.

### FIELD OF THE INVENTION

The present invention relates in general to the field of genetic diagnosis and therapy and, more particularly, to the characterization and use of an atonal-associated nucleic acid or amino acid sequence, or any of its homologs or orthologs, as a therapeutic agent for the treatment of a gastrointestinal condition.

### BACKGROUND OF THE INVENTION

An intricate pattern of interactions within and between cells directs the sequential development of neurons from dividing neuroepithelial progenitor cells. Multiple extracellular and intracellular signals moderate this process. Among the key intracellular signals are transcription factors, which induce the expression of a cascade of genes. One subclass of transcription factors, belonging to the basic helix-loop-helix (bHLH) family of proteins, is expressed early on when the decision to proliferate or differentiate is made. This function is a particularly crucial one as mutations in these genes early in development can wipe out entire neural structures.

In Drosophila, the gene atonal (ato), which is homologous to Math1, Math2, Hath1 and Hath2, encodes a bHLH protein essential for the development of chordotonal organs (sensory organs found in the body wall, joints and antenna that function in proprioception, balance and audition) (Eberl, 1999; McIver, 1985; van Staaden and Römer, 1998). CHOs populate the peripheral nervous system (PNS) in the body wall and joints (thorax, abdomen, sternum, wings, legs) and antennae (Moulins, 1976), providing the fly with sensory information much as touch and mechanoreceptors do in vertebrates (McIver, 1985; Moulins, 1976). Boyan (Boyan, 1993) proposed that, in the course of evolution, different CHOs became specialized for hearing in different insects. This hypothesis was recently confirmed by van Staaden and Römer (1998). In Drosophila, CHOs in the Johnston organ, located in the second antennal segment, function in near field hearing (Dreller and Kirschner, 1993; Eberl, 1999) and negative geotaxis.

During development ato is expressed in a cluster of progenitor cells from which the CHO founder cells are selected (Jarman et al., 1993). It likely functions by regulating the expression of genes necessary for the specification and development of the CHO lineage; as it encodes a basic helix-loop-helix protein (bHLH) that dimerizes with the Daughterless protein and binds to E-box sequences, thereby activating genes (Jarman et al., 1993). CHO specificity is encoded by the ato basic domain, which is required for DNA binding in bHLH proteins (Chien et al., 1996; Davis et al., 1990; Jarman and Ahmed, 1998; Vaessin et al., 1990). ato is both necessary and sufficient for the generation of CHOs in the fly: loss of ato function leads to the loss of CHOs, while ectopic ato expression causes ectopic CHO formation (Jarman et al., 1993). Adult flies that lack atonal function are uncoordinated, do not fly, and are deficient in hearing. Overexpression of the fly atonal gene can generate new chordotonal neurons, indicating that atonal is both essential and sufficient for the development of this neuronal population.

In vertebrates, during myogenesis and neurogenesis, cell fate specification requires basis helix-loop-helix (bHLH) transcription factors. Math1 (for mouse atonal homolog-1) is such a factor, and is expressed in the hindbrain, dorsal spinal cord, external germinal layer of the cerebellum, gut, joints, ear and Merkel cells of the skin (which function as mechanoreceptors) (Akazawa et al.,1995; Ben-Arie et al., 1996; Ben-Arie et al., 1997). Mice heterozygous for a targeted deletion of Math1 (Math1^{+/-}) are viable and appear normal, but Math1 null mice (Math1^{-/-}) die shortly after birth and lack cerebellar granule neurons.

Math1 is one of ato's closest known homologs, with 82% amino acid similarity in the bHLH domain and 100% conservation of the basic domain that determines target recognition specificity (Ben-Arie et al., 1996; Chien et al., 1996). Math1 is transiently expressed in the CNS starting at embryonic day 9 (E9) in the dorsal portion of the neural tube. Math1 is also expressed in the rhombic lip of the fourth ventricle of the brain, where cerebellar granule cell precursors are born at E13-15 (Alder et al., 1996). Upon proliferation and differentiation, these progenitor cells migrate to form the external granule layer (EGL) of the cerebellar primordia (Hatten and Heintz, 1995). Proliferating EGL cells continue to express Math1 during the first three postnatal weeks, until shortly before they migrate to their final adult destination to generate the internal granule layer (IGL) of the cerebellum (Akazawa et al., 1995; Ben-Arie et al., 1996). Another group of cells, a small population of neuronal precursors in the dorsal spinal cord, expresses Math1 during E10-E14 (Akazawa et al., 1995; Ben-Arie et al., 1996). These precursor cells also express the LIM homeodomain proteins (LH2A and LH2B), markers of the D1 class of commissural interneurons (Lee et al., 1998). Helms and Johnson (1998) reported that lacZ expression under the control of Math1 regulatory elements reproduced Math1 expression patterns in the developing cerebellum and spinal cord, and demonstrated that Math1 is expressed in precursors that give rise to a subpopulation of dorsal commissural interneurons.

To determine the in vivo function of Math1, the inventors generated mice (Math1^{-/-}) lacking the MATH1 protein. This null mutation causes major cerebellar abnormalities: lack of granule cell proliferation and migration from the rhombic lip at E14.5, and absence of the entire EGL at birth (Ben-Arie et al., 1997). It is not clear whether the agenesis of cerebellar granule neurons is due to failure of progenitor specification or the cells' inability to proliferate and/or differentiate. Neonates cannot breathe and die shortly after birth, but there are no gross defects in any cranial nerves or brain stem nuclei that could explain respiratory failure.

The fact that Math1 is expressed in the inner ear suggests that Math1expression is necessary for the development of auditory or balance organs. The inner ear initially forms as a thickening of the ectoderm, termed the otic placode, between rhombomeres 5 and 6 in the hindbrain. The otic placode gives rise to neurons of the VIIIth cranial nerve and invaginates to become the otocyst, from which the inner ear will develop. The mature mammalian inner ear comprises one auditory organ, the cochlea, and five vestibular organs: the utricle, the saccule, and three semicircular canals. The sensory epithelia of these organs consist of mechanoreceptive hair cells, supporting cells and nerve endings. Hair cells serve as mechanoreceptors for transducing sound waves and head motion into auditory and positional information. Hair cells and supporting cells both arise from a common progenitor cell and proliferate and differentiate within the sensory epithelia, with peak mitoses between embryonic day 13 and 18 (E13-18) in mice. Although several genes have been implicated in the development of the inner ear, such as int2 (Mansour et al., 1993; pax2 (Torres et al., 1996; and Hmx3 (Wang et al., 1998). None have been shown to be required for the genesis of hair cell specifically.

Damage to hair cells is a common cause of deafness and vestibular dysfunction, which are themselves prevalent diseases. Over 28 million Americans have impaired hearing; vestibular disorders affect about one-quarter of the general population, and half of our elderly. The delicate hair cells are vulnerable to disease, aging, and environmental trauma (i.e., antibiotics, toxins, persistent loud noise). Once these cells are destroyed, they cannot regenerate in mammals. Therefore, a need exists to address the problems of patients with congenital, chronic or acquired degenerative hearing impairment and loss or balance problems, and to provide compositions, methods and reagents for use in treating hearing loss and vestibular function.

In support of the teaching of the present invention, others have demonstrated that Math1, upon overexpression, induces significant production of extra hair cells in postnatal rat inner ears (Zheng and Gao, 2000). Briefly, although fate determination is usually completed by birth for mammalian cochlear hair cells, overexpression of Math1 in postnatal rat cochlear explant cultures results in additional ear hair cells which derive from columnar epithelial cells located outside the sensory epithelium in the greater epithelial ridge. Furthermore, conversion of postnatal utricular supporting cells into hair cells is facilitated by Math1 expression. The ability of Math1 to permit production of hair cells in the ear is strong evidence in support of the claimed invention.

In addition to Math1 being involved in governing differentiation of neuronal cells, including sensory cells in the inner ear, Math1 is involved in intestinal development, as described herein. The mouse gut begins developing at embryonic day 7.5 (E7.5). Invagination of the most anterior and posterior endoderm leads to the formation of the foregut and hindgut pockets, respectively, which extend toward each other and fuse to form the gut tube. By E15.5, the gut appears as a poorly differentiated, pseudostratified epithelium. From E15.5 to E19, nascent villi with a monolayer of epithelial cells develop in a duodenum-to-colon pattern. During the first two postnatal weeks, the intervillus epithelium, where proliferating and less differentiated cells reside, develops into the crypts of Lieberkühn. Stem cells in the intervillus epithelium (during embryogenesis) or crypts (in adulthood) give rise to four principle cell types: absorptive enterocytes or columnar cells, mucous-secreting goblet cells, regulatory peptide-secreting enteroendocrine cells in the large and small intestines, and antimicrobial peptide-secreting Paneth cells in the small intestine only. Enterocytic, goblet, and enteroendocrine cells continue to differentiate and mature while migrating up the villus, and are finally extruded into the lumen at the tip. This journey takes about 2 to 3 days. The Paneth cells migrate downward and reside at the base of the crypt for 21 days before being cleared by phagocytosis (Cheng and Leblond, 1974; Gordon et al., 1992; Back et al., 2000).

The epithelial-mesenchymal interaction has been shown to be critical in the proximal-distal, crypt-villus patterning during gut development. A number of signaling molecules and transcription factors are involved in these processes (Kaestner et al., 1997; Pabst et al., 1999; Beck et al., 2000; Clatworthy and Subramanian, 2001). Previous studies have suggested that all four epithelial cell lineages originate from a common ancestor (Cheng and Leblond, 1974; Gordon et al., 1992; Back et al., 2000; Bjerknes and Cheng, 1999), but the mechanisms that control the epithelial lineage differentiation are not well understood. T cell factor-4 (Tcf4) plays a role in the stem cell maintenance in the small intestine but does not induce epithelial cells to differentiate into enterocytes or goblet cells (Korlinek et al., 1998). The present invention is directed to methods and compositions utilizing Math1, particularly in gut development, as it is expressed in the gut (Akazawa et al., 1995) in addition to being involved in cell fate determination in the nervous system (Ben-Arie et al., 2000; Bermingham et al., 2001).

### SUMMARY OF THE INVENTION

In one embodiment of the present invention there is an animal having a heterologous nucleic acid sequence replacing an allele of an atonal-associated nucleic acid sequence under conditions wherein said heterologous sequence inactivates said allele. In a preferred embodiment said heterologous sequence is expressed under control of an atonal-associated regulatory sequence. In a specific embodiment both atonal-associated alleles are replaced. In an additional specific embodiment both atonal-associated alleles are replaced with nonidentical heterologous nucleic acid sequences. In an additional embodiment said animal has a detectable condition wherein said condition is selected from the group consisting of loss of hair cells, cerebellar granule neuron deficiencies, hearing impairment, imbalance, joint disease, osteoarthritis, abnormal proliferation of neoplastic neuroectodermal cells and formation of medulloblastoma. In another embodiment of the present invention said heterologous nucleic acid sequence is a reporter sequence selected from the group consisting of b-galactosidase, green fluorescent protein (GFP), blue fluorescent protein (BFP), neomycin, kanamycin, luciferase, β-glucuronidase and chloramphenicol transferase (CAT). In another specific embodiment said reporter sequence regulatable or is expressed in brain tissue, neural tissue, skin tissue, non-ossified cartilage cells, joint chondrocytes, Merkel cells, iriner ear sensory epithelia and brain stem nuclei. In additional specific embodiments said atonal-associated allele is replaced with an atonal-associated nucleic acid sequence under control of a regulatable promoter sequence or a tissue-specific promoter sequence wherein said tissue is selected from the group consisting of brain tissue, neural tissue, skin tissue, non-ossified cartilage cells, joint chondrocytes, Merkel cells, inner ear sensory epithelia and brain stem nuclei. In additional embodiments said animal is a mouse, Drosophila, zebrafish, frog, rat, hamster or guinea pig.

In another embodiment of the present invention is a method for screening for a compound in an animal, wherein said compound affects expression of an atonal-associated nucleic acid sequence comprising delivering said compound to said animal wherein said animal has at least one allele of an atonal-associated nucleic acid sequence inactivated by insertion of a heterologous nucleic acid sequence wherein said heterologous nucleic acid sequence is under control of an atonal-associated regulatory sequence, and monitoring for a change in said expression of said atonal-associated nucleic acid sequence. In specific embodiments said compound upregulates or downregulates said expression of an atonal-associated nucleic acid sequence. In additional embodiments said animal is a mouse or Drosophila. In a specific embodiment the heterologous nucleic acid sequence is a reporter sequence. In an additional specific embodiment the heterologous nucleic acid sequence is selected from the group consisting of β-galactosidase, green fluorescent protein (GFP), blue fluorescent protein (BFP), neomycin, kanamycin, luciferase, β-glucuronidase and chloramphenicol transferase (CAT).

Another embodiment of the present invention is a compound which affects expression of an atonal-associated nucleic acid sequence. In specific embodiments said compound upregulates or downregulates expression of an atonal-associated nucleic acid sequence. In a specific embodiment said compound affects a detectable condition in an animal wherein said condition is selected from the group consisting of loss of hair cells, cerebellar granule neuron deficiencies, hearing impairment, an imbalance disorder, joint disease, osteoarthritis, abnormal proliferation of neoplastic neuroectodermal cells and formation of medulloblastoma.

Another embodiment of the present invention is a method for screening for a compound in an animal, wherein said compound affects a detectable condition in said animal, comprising delivering said compound to said animal wherein at least one allele of an atonal-associated nucleic acid sequence in said animal is inactivated by insertion of a heterologous nucleic acid sequence, wherein said heterologous nucleic acid sequence is under the control of an atonal-associated regulatory sequence, and monitoring said animal for a change in the detectable condition. In a specific embodiment said detectable condition is selected from the group consisting of loss of hair cells, cerebellar granule neuron deficiencies, hearing impairment, an imbalance disorder, joint disease, osteoarthritis, abnormal proliferation of neoplastic neuroectodermal cells and formation of medulloblastoma. In another embodiment said delivery of said compound affects expression of said heterologous nucleic acid sequence. In specific embodiments said expression of said heterologous nucleic acid sequence is upregulated or downregulated. In additional specific embodiments said animal is a mouse, Drosophila, zebrafish, frog, rat, hamster or guinea pig.

Another embodiment of the present invention is a compound wherein said compound affects said detectable condition. In specific embodiments said compound affects expression of a heterologous nucleic acid sequence. In additional specific embodiments said compound upregulates or downregulates expression of a heterologous nucleic acid sequence.

In other embodiments of the present invention are methods of treating an animal, including a human, for cerebellar granule neuron deficiencies, for promoting mechanoreceptive cell growth, for generating hair cells, for treating hearing impairment or an imbalance disorder, for treating a joint disease, for treating for an abnormal proliferation of cells, and for treating for a disease that is a result of loss of functional atonal-associated nucleic acid or amino acid sequence. Said methods include administering a therapeutically effective amount of an atonal-associated nucleic acid or amino acid sequence. In specific embodiments said administration is by a vector selected from the group consisting of an adenoviral vector, a retroviral vector, an adeno-associated vector, a plasmid, or any other nucleic acid based vector, a liposome, a nucleic acid, a peptide, a lipid, a carbohydrate and a combination thereof of said vectors. In a specific embodiment said vector is a non-viral vector or a viral vector. In another specific embodiment said vector is a cell. In a preferred embodiment said vector is an adenovirus vector comprising a cytomegalovirus IE promoter sequence and a SV40 early polyadenylation signal sequence. In another specific embodiment said cell is a human cell. In an additional specific embodiment said joint disease is osteoarthritis. In a specific embodiment said atonal-associated nucleic acid or amino acid sequence is Hath1 or Math1. In another specific embodiment the cell contains an alteration in an atonal-associated nucleic acid or amino acid sequence. In an additional specific embodiment said amino acid sequence has at least about 80% identity to about 20 contiguous amino acid residues of SEQ ID NO:58. In an additional specific embodiment the nucleic acid sequence encodes a polypeptide which has at least about 80% identity to about 20 contiguous amino acid residues of SEQ ID NO:58.

In another embodiment of the present invention is a method for treating an animal for an abnormal proliferation of cells comprising altering atonal-associated nucleic acid or amino acid sequence levels in a cell. In a specific embodiment said alteration is reduction or said nucleic acid or amino acid sequence contains an alteration.

In another embodiment of the present invention is a composition comprising an atonal-associated amino acid sequence or nucleic acid sequence in combination with a delivery vehicle wherein said vehicle delivers a therapeutically effective amount of an atonal-associated nucleic acid sequence or amino acid sequence into a cell. In specific embodiments said vehicle is the receptor-binding domain of a bacterial toxin or any fusion molecule or is a protein transduction domain. In a specific embodiment said protein transduction domain is from the HIV TAT peptide. In a specific embodiment said atonal-associated amino acid sequence or nucleic acid sequence is Hath1 or Math1.

In another embodiment of the present invention there is a composition to treat an organism for loss of hair cells, wherein said organism comprises a defect in an atonal-associated nucleic acid sequence. In a specific embodiment the defect is a mutation or alteration of said atonal-associated nucleic acid sequence. In another specific embodiment the defect affects a regulatory sequence of said atonal-associated nucleic acid sequence. In an additional embodiment of the present invention there is a composition to treat an organism for loss of hair cells, wherein said organism comprises defect in a nucleic acid sequence which is associated with regulation of an atonal-associated nucleic acid sequence. In an additional embodiment of the present invention there is a composition to treat an organism for loss of hair cells, wherein said organism comprises a defect in an amino acid sequence which is associated with regulation of an atonal-associated nucleic acid sequence.

In another embodiment of the present invention there is a composition to treat an organism for a cerebellar neuron deficiency, wherein said organism comprises a defect in an atonal-associated nucleic acid sequence. In a specific embodiment the defect is a mutation or alteration of said atonal-associated nucleic acid sequence. In another specific embodiment the defect affects a regulatory sequence of said atonal-associated nucleic acid sequence. In an additional embodiment of the present invention there is a composition to treat an organism for a cerebellar neuron deficiency, wherein said organism comprises defect in a nucleic acid sequence which is associated with regulation of an atonal-associated nucleic acid sequence. In an additional embodiment of the present invention there is a composition to treat an organism for a cerebellar neuron deficiency, wherein said organism comprises a defect in an amino acid sequence which is associated with regulation of an atonal-associated nucleic acid sequence.

In another embodiment of the present invention there is a composition to treat an organism for hearing impairment, wherein said organism comprises a defect in an atonal-associated nucleic acid sequence. In a specific embodiment the defect is a mutation or alteration of said atonal-associated nucleic acid sequence. In another specific embodiment the defect affects a regulatory sequence of said atonal-associated nucleic acid sequence. In an additional embodiment of the present invention there is a composition to treat an organism for hearing impairment, wherein said organism comprises defect in a nucleic acid sequence which is associated with regulation of an atonal-associated nucleic acid sequence. In an additional embodiment of the present invention there is a composition to treat an organism for hearing impairment, wherein said organism comprises a defect in an amino acid sequence which is associated with regulation of an atonal-associated nucleic acid sequence.

In another embodiment of the present invention there is a composition to treat an organism for imbalance, wherein said organism comprises a defect in an atonal-associated nucleic acid sequence. In a specific embodiment the defect is a mutation or alteration of said atonal-associated nucleic acid sequence. In another specific embodiment the defect affects a regulatory sequence of said atonal-associated nucleic acid sequence. In an additional embodiment of the present invention there is a composition to treat an organism for imbalance, wherein said organism comprises defect in a nucleic acid sequence which is associated with regulation of an atonal-associated nucleic acid sequence. In an additional embodiment of the present invention there is a composition to treat an organism for imbalance, wherein said organism comprises a defect in an amino acid sequence which is associated with regulation of an atonal-associated nucleic acid sequence.

In another embodiment of the present invention there is a composition to treat an organism for osteoarthritis, wherein said organism comprises a defect in an atonal-associated nucleic acid sequence. In a specific embodiment the defect is a mutation or alteration of said atonal-associated nucleic acid sequence. In another specific embodiment the defect affects a regulatory sequence of said atonal-associated nucleic acid sequence. In an additional embodiment of the present invention there is a composition to treat an organism for osteoarthritis, wherein said organism comprises defect in a nucleic acid sequence which is associated with regulation of an atonal-associated nucleic acid sequence. In an additional embodiment of the present invention there is a composition to treat an organism for osteoarthritis, wherein said organism comprises a defect in an amino acid sequence which is associated with regulation of an atonal-associated nucleic acid sequence.

In another embodiment of the present invention there is a composition to treat an organism for a joint disease, wherein said organism comprises a defect in an atonal-associated nucleic acid sequence. In a specific embodiment the defect is a mutation or alteration of said atonal-associated nucleic acid sequence. In another specific embodiment the defect affects a regulatory sequence of said atonal-associated nucleic acid sequence. In an additional embodiment of the present invention there is a composition to treat an organism for a joint disease, wherein said organism comprises defect in a nucleic acid sequence which is associated with regulation of an atonal-associated nucleic acid sequence. In an additional embodiment of the present invention there is a composition to treat an organism for a joint disease, wherein said organism comprises a defect in an amino acid sequence which is associated with regulation of an atonal-associated nucleic acid sequence.

In another embodiment of the present invention there is a composition to treat an organism for abnormal proliferation of cells, wherein said organism comprises a defect in an atonal-associated nucleic acid sequence. In a specific embodiment the defect is a mutation or alteration of said atonal-associated nucleic acid sequence. In another specific embodiment the defect affects a regulatory sequence of said atonal-associated nucleic acid sequence. In an additional embodiment of the present invention there is a composition to treat an organism for abnormal proliferation of cells, wherein said organism comprises defect in a nucleic acid sequence which is associated with regulation of an atonal-associated nucleic acid sequence. In an additional embodiment of the present invention there is a composition to treat an organism for abnormal proliferation of cells, wherein said organism comprises a defect in an amino acid sequence which is associated with regulation of an atonal-associated nucleic acid sequence.

In another embodiment of the present invention there is a composition to treat an organism for cancer, wherein said organism comprises a defect in an atonal-associated nucleic acid sequence. In a specific embodiment the defect is a mutation or alteration of said atonal-associated nucleic acid sequence. In another specific embodiment the defect affects a regulatory sequence of said atonal-associated nucleic acid sequence. In an additional embodiment of the present invention there is a composition to treat an organism for cancer, wherein said organism comprises defect in a nucleic acid sequence which is associated with regulation of an atonal-associated nucleic acid sequence. In an additional embodiment of the present invention there is a composition to treat an organism for cancer, wherein said organism comprises a defect in an amino acid sequence which is associated with regulation of an atonal-associated nucleic acid sequence. In a specific embodiment said cancer is medulloblastoma.

In an object of the present invention, there is a method of predicting a differentiation state for a stem cell, comprising the steps of obtaining the cell; and determining the expression status of an atonal-associated sequence. In a specific embodiment, the stem cell is an intestinal stem cell. In another specific embodiment, the stem cell is obtained from an intestinal epithelium. In an additional specific embodiment, the expression status of the atonal-associated sequence is an upregulation of expression of the atonal-associated sequence. In another specific embodiment, the differentiation state is to a secretory cell of the intestine. In a specific embodiment, the secretory cell is at least one of a goblet cell, an enteroendocrine cell, or a Paneth cell. In an additional specific embodiment, the expression status of said atonal-associated sequence is a downregulation of expression of said atonal-associated sequence. In another specific embodiment, the differentiation state is to an absorptive cell of the intestine. In a further specific embodiment, the atonal-associated sequence is a polynucleotide. In another specific embodiment, the atonal-associated sequence is a polypeptide.

In another object of the present invention, there is a method for differentiating a stem cell, comprising altering expression of an atonal-associated sequence. In a specific embodiment, the stem cell is a gastrointestinal stem cell. In an additional specific embodiment, the stem cell differentiates into a secretory cell. In a specific embodiment, the secretory cell is at least one of a goblet cell, an enteroendocrine cell, or a Paneth cell. In a further specific embodiment, the stem cell differentiates into an absorptive cell.

In an additional object of the present invention, there is a method of regenerating secretory intestinal cells in an individual, comprising the step of administering to the individual a stem cell and a regulatory factor for said stem cell, wherein the expression of an atonal-associated sequence is upregulated in the stem cell. In a specific embodiment, the secretory intestinal cell is at least one of a goblet cell, an enteroendocrine cell, or a Paneth cell. In another specific embodiment, the regulatory factor is a bone morphogenetic protein. In a specific embodiment, the bone morphogenetic protein is GDF7.

In another object of the present invention, there is a method of regenerating absorptive intestinal cells in an individual, comprising the step of administering to the individual a stem cell and a regulatory factor for said stem cell, wherein the expression of an atonal-associated sequence is downregulated in the stem cell. In a specific embodiment, the regulatory factor is a member of the HES family.

In an additional object of the present invention there is a method of treating an animal for a gastrointestinal condition, comprising delivering to the animal a gastrointestinal stem cell. In a specific embodiment, the method further comprises delivery of a regulatory factor.

In another object of the present invention there is a method of treating an animal for a gastrointestinal condition comprising delivering a therapeutically effective amount of an atonal-associated amino acid sequence or nucleic acid sequence to a cell of said animal. In a specific embodiment, the gastrointestinal condition is cancer, damaged intestinal tissue, inflammatory bowel disease, irritable bowel syndrome, infection or necrotizing entercolitis. In a specific embodiment, the atonal-associated amino acid sequence or nucleic acid sequence is Math1. In another specific embodiment, the atonal-associated amino acid sequence or nucleic acid sequence is Hath1. In a further specific embodiment, the amino acid sequence or nucleic acid sequence is administered by a delivery vehicle. In a further specific embodiment, the delivery vehicle is an adenoviral vector, a retroviral vector, an adeno-associated viral vector, a plasmid, a liposome, a nucleic acid sequence, a peptide, a lipid, a carbohydrate or a combination thereof. In an additional specific embodiment, the delivery vehicle is selected from the group consisting of a viral vector or a non-viral vector. In another specific embodiment, the cell contains an alteration in an atonal-associated nucleic acid sequence or amino acid sequence. In an additional specific embodiment, the amino acid sequence has at least about 80% identity to about 20 contiguous amino acid residues of SEQ ID NO:58 (Hath1). In a further specific embodiment, the nucleic acid sequence encodes a polypeptide which has at least about 80% identity to about 20 contiguous amino acid residues of SEQ ID NO:58 (Hath1).

In another object of the present invention, there is a composition in a pharmaceutical carrier, comprising at least one stem cell, wherein the cell is upregulated for expression of an atonal-associated sequence; and at least one regulatory factor. In a specific embodiment, the stem cell is a gastrointestinal stem cell.

In an additional specific embodiment, the composition in a pharmaceutical carrier, comprising at least one stem cell, wherein the cell is downregulated for expression of an atonal-associated sequence; and at least one regulatory factor. In a specific embodiment, the stem cell is a gastrointestinal stem cell.

In another object of the present invention, there is a method of treating an individual for a gastrointestinal condition, comprising the step of administering to the individual a composition described herein.

In an additional object of the present invention, there is a method for screening for a compound in an animal, wherein said compound affects a detectable gastrointestinal condition in said animal, comprising delivering the compound to said animal wherein at least one allele of an atonal-associated nucleic acid sequence in said animal is inactivated by insertion of a heterologous nucleic acid sequence, wherein said heterologous nucleic acid sequence is under the control of an atonal-associated regulatory sequence, and monitoring said animal for a change in the detectable gastrointestinal condition. In a specific embodiment, the delivery of the compound affects expression of the heterologous nucleic acid sequence. In a specific embodiment, the compound affects the detectable condition.

In another object of the present invention, there is a kit comprising an intestinal stem cell. In a specific embodiment, the kit further comprises a regulatory protein.

In an additional object of the present invention, there is a method of treating an animal for a disease that is a result of loss of functional atonal-associated nucleic acid or amino acid sequence comprising delivering a therapeutically effective amount of an atonal-associated amino acid sequence or nucleic acid sequence to a cell of said animal. In a specific embodiment, the disease is a gastrointestinal disease.

Other and further objects, features and advantages would be apparent and eventually more readily understood by reading the following specification and by reference to the company drawing forming a part thereof, or any examples of the presently preferred embodiments of the invention are given for the purpose of the disclosure.

### DESCRIPTION OF THE DRAWINGS

FIG. 1A and 1B demonstrate that the inner ear β-Gal staining (blue) of Math1 heterozygous embryos as described hereinabove. FIG. 1A shows the otic vesicle (OV) at E12.5 and FIG. 1B the inner ear at E14.5 of Math1^{+/β-Gal} embryos. Sensory epithelia stained positively in the cochlea (C), saccule (S), utricle (U), and semicircular canal ampullae (SCA). A schematic diagram of the inner ear is depicted alongside the staining for reference, blue indicates location of the sensory epithelia. The original magnifications of the images taken under the microscope were ×100 for FIG. 1A and ×50 for FIG. 1B.

FIGS. 2A through 2F are scanning electron micrographs of E18.5 inner ear sensory epithelia in wild-type and Math1^{β-Gal/β-Gal} mice. Wild-type mice epithelia are shown in FIGS. 2A, 2C, and 2E and null mouse epithelia in FIGS. 2B, 2D, and 2F. The organ of Corti of the cochlea are shown and indicated in FIGS. 2A and 2B. In the wild-type mouse there are three rows of outer hair cells (1, 2, 3), one row of inner hair cells (I), all with hair bundles (HB). The tectorial membrane (TM), an accessory structure of the cochlea, can be observed at the bottom. Above the sensory epithelium are squamous cells (SQ) with rudimentary kinocilia (RK). In null mice (FIG. 2B), there are only squamous cells. Crista ampullaries of a vertical semicircular canal are depicted in FIGS. 2C and 2D. The null mouse crista is similar to the wild-type in overall shape, including the septum (eminentia) cruciatum (EC), but is smaller. The macula of the uticle is the focus of FIGS. 2E and 2F. Again, the macula of the null mouse is smaller than the wild-type. Scale bars are as follows: 10 μm in FIGS. 2A and 2B, 50 μm in FIGS. 2C and 2D, and 100 μm in FIGS. 2E and 2F.

FIGS. 3A through 3F are light micrographs of semi-thin transverse sections of inner ear sensory epithelia in wild-type mice (FIGS. 3A, 3C, and 3E) and Math1^{β-Gal/β-Gal} (FIGS. 3B, 3D, and 3F), all mice were observed at E18.5. As observed in the cochlea of wild-type mice, FIG. 3A, three outer hair cells (1, 2, 3) and one inner (I) hair cell are present. Conversely, the null mouse cochlea (FIG. 3B) has only squamous cells (SQ) in the same region. Hair cells (HC) and supporting cells (SC) are present in the wild-type crista ampullaris (FIG. 3C) and utricular macula (FIG. 3E), but only supporting cells are present in null mice (FIG. 3D and 3F). The crista was cut obliquely, accounting for the multiple layers of hair cells in FIG. 3C. The otolithic membrane (OM), an accessory structure of the utricle, is present in both wild-type mice (FIG. 3E) and null mice (FIG. 3F). Scale bars equal 100 μm in (FIGS. 3A and 3B); 50 μm in (FIGS. 3C and 3D); and 25 μm in (FIGS. 3E and 3F).

FIGS. 4A and 4B are transmission electron micrographs of E18.5 utricular macula in wild-type and Math1^{β-Gal/β-Gal} mice. FIG. 4A shows that the hair cells (HC) and supporting cells (SC) are present in wild-type utricular macula. By contrast, only supporting cells are present in the null mouse (FIG. 4B). Hair cells have hair bundles (HB) and supporting cells have miicrovilli (MV). Hair cells are less electron-dense and have more apical nuclei than supporting cells, but only the latter have secretory granules (SG). Some immature hair cells (IM) are evident in the wild-type, but not in the null mouse. The scale bar in all the figures equals 10 μm.

FIGS. 5A through 5F show the Calretinin staining pattern of inner ear sensory epithelia. Sections through the utricle of E16.5 wild-type (FIGS. A5 and 5C) and Math1^{β-Gal/β-Gal} (FIGS. 5B and 5D) littermates were counterstained with propidium iodide (red) for confocal microscopy. Sections were cut through the crista ampullaris of E18.5 wild-type (FIG. 5E) and Math1^{β-Gal/β-Gal} (FIG. 5F) were counterstained with DAPI (blue) for immunofluorescent microscopy. The crista is cut at an oblique angle, which accounts for the multiple layers of hair cells in (FIG. 5E). Immunostaining of Calretinin (green, arrows) is evident in hair cells of wild-type (FIGS. 5A, 5C, and 5E) but not null mice (FIGS. 5B, 5D, and 5F). Boxed areas in FIGS. 5A and 5B indicate the regions magnified in FIGS. 5C and 5D. Scale bar equals 100 μm in (FIGS. 5A and 5B), 15 μm in (FIGS. 5C and 5D) and an original magnification of ×200 in (FIGS. 5E and 5F).

FIGS. 6A and 6B show the expression pattern of Math1 in mouse articular cartilage using the Math1^{+/β-Gal} heterozygote. FIG. 6A shows the staining pattern of a P14 mouse forelimb and demonstrates expression in all joints. FIG. 6B is a magnification (20×) of an elbow joint from the same mouse that demonstrates that Math1 is expressed exclusively in the non-ossified articular chondrocytes.

FIGS. 7A through 7C show replacement of Math1 coding region by lacZ gene. FIG. 7A, Top, has a map of the Math1 genomic locus. The coding region is shown as a black box. The sites of the probes used to detect the wild-type and mutant alleles are shown as black bars. The targeting vector is in the middle with the sites for homologous recombination indicated by larger Xs. In the targeted locus shown at the bottom, lacZ is translated under the control of Math1 regulatory elements. FIG. 7B demonstrates Southern blot analysis of embryonic stem cells using the 3′ external probe. The upper band represents wt allele and the lower band the targeted mutant allele (mut) in targeted clones. FIG. 7C demonstrates Southern blot analysis of DNA from the progeny of heterozygous mice demonstrating the presence of the targeted allele and absence of the wild-type allele in Math1^{β-gal/β-gal} mice (asterisks). The abbreviations are as follows: (A) ApaI; (H) HindIII; (RI) EcoRI; (S) SalI; and (X) XbaI.

FIGS. 8A through 8H show Math1/lacZ expression and cerebellar phenotype in Math1^{+/β-gal} and Math1^{β-gal/β-gal} mice. FIG. 8A shows Math1/lacZ expression in the dorsal neural tube at E9.5 and (FIG. 8B) E10.5. FIG. 8C indicates a section through the hind brain at E10.5 has Math1/lacZ expression in the dorsal portion (arrows). FIG. 8D demonstrates that in a spinal cord section from E12.5 embryo, dorsal cells migrate ventrally (arrows). FIG. 8E shows at E14.5 expression is observed in the EGL progenitors at the rhombic lip and in migrating cells that will populate the EGL. FIG. 8F demonstrates in Math1^{β-gal/β-gal} mice, Math1/lacZ expression is limited to a few cells in the rhombic lip, which is significantly reduced in size. FIG. 8G shows that at P0 Math1/lacZ is expressed in the EGL. FIG. 8H demonstrates that the EGL is absent in the null mice. Original magnification for FIGS. 8C through 8H was 100×.

FIGS. 9A through 9G shows expression of Math1/lacZ in the inner ear and brain stem and histological analysis of ventral pontine nucleus. X-gal staining of E18.5 Math1^{+/β-gal} utricular crista (FIG. 9A) and inner ear sensory epithelia of Math1^{+/β-gal} (FIG. 9B) and Math1^{β-gal/β-gal} (FIG. 9C). The Math1/lacZ expression in the upper hair cell layer of the sensory epithelia of (FIGS. 9A and 9B) and the characteristic calyx appearance (arrowhead). In the null mice X-gal staining of epithelial cells is non-specific in the absence of hair cells (FIG. 9C). Whole-brain X-gal staining of Math1^{+/β-gal} (FIG. 9D) and Math1^{β-gal/β-gal} (FIG. 9E) at E18.5 is demonstrated. There is positive staining of the pontine nucleus (arrowhead) and cerebellum (arrow) in Math1^{+/β-gal} mice, which is lacking or greatly reduced in null mutants in both the cerebellum, and pontine nucleus (inset). FIGS. 9F and 9G show haematoxylin and eosin staining of sagittal sections through the pons of a wild type and null mutant (FIGS. 9F and 9G, respectively), showing the loss of the ventral pontine nucleus in null mutants. The original magnifications were as follows: (A) 400× (B & C) 1000×, (D & E) 8×, inset in D & E 100×, (F & G) 10×.

FIGS. 10A through 10E demonstrate Math1/lacZ is expressed in joint chondrocytes. X-gal staining of whole embryos at (FIG. 10A) E12.5 and (FIG. 10B) E16.5 illustrates that Math1/lacZ is expressed in all joints (FIG. 10C). Horizontal section through the elbow joint of E18.5 Math1^{+/β-gal} mouse shows that it is expressed in resting chondrocytes (arrow). FIG. 10D shows a horizontal section through a humero-radial joint at P10 that has expression in the articular chondrocytes (arrowhead) and resting chondrocytes (arrow). FIG. 10E shows high magnification of a section through a wrist joint indicating Math1/lacZ is expressed in articular chondrocytes. The original magnification is as follows: (C) 10×; (D) 20×; and (E) 40×.

FIGS. 11A through 11L show Math1/lacZ expression in Merkel cells. To identify the structures stained on the hairy and non-hairy skin, E16.5 littermate embryos were stained as whole mounts, sectioned, and microscopically examined. Shown are sections through the vibrissae (FIG. 11A), foot pad at low (FIG. 11B) and high magnification of the region marked by an arrow in B (FIG. 10C), and hairy skin (FIG. 11D). In all sections the localization of the stained cells was as expected from Merkel cells. To look for macroscopic defects in null mice, close-up pictures were taken through a stereomicroscope of Math1^{+/β-gal} (control, panels E-H) and Math1^{β-gal/β-gal} (null, panels I-L) littermate mice. Staining in null mice appeared stronger because of a dosage effect in the vibrissae (E, I), limb joints (F, J), and foot pads (G, K). In contrast, the staining intensity of null (J, L) mice was markedly weaker than that of heterozygous (F, H) mice in the touch domes associated with the hairy skin. The original magnification was follows: A ×200; B ×50; C ×400; D ×500; EG-H-I-K-L ×32; F-J ×16.

FIGS. 12A through 12E show lack of lacZ-stained touch domes in Tabby mice. Tabby/Tabby females were crossed with Math1^{+/β-gal} males, and their progeny were X-gal stained and gender-determined at E16.5. Staining around primary vibrissae in the snout was detected in both female embryos heterozygous for the Tabby mutation (FIG. 12A) and male embryos hemizygous for the mutation (FIG. 12B). Secondary vibrissae, which are known to vary in number in the Tabby mutants (black arrows), were also stained. The staining of the touch domes was less intense in the Tabby/X female (FIG. 12D) than Math1^{+/β-gal} (wt for Tabby) embryos (FIG. 12C), since Tabby is a semidominant mutation. However, patches of stained touch domes were detected in a female embryo that carried a wild-type allele at the Tabby locus (FIG. 12A, red arrow, and 12D). In contrast, a hemizygous male completely lacked both staining and touch domes, due to the loss of hair follicles that abolishes the development of Merkel cells (FIGS. 12B and 12E).

FIGS. 13A through 13F demonstrate marker analysis of Merkel cells in wild type and Math1 null mice. Skin sections from Math1^{+/+} and Math1^{β-gal/β-gal} reacted with antibodies against MATH1 (FIGS. 13A and 13B), cytokeratin 18 (FIGS. 13C and 13D), and chromogranin A (FIGS. 13E and 13F). Polyclonal antibodies to MATH1 identify multiple basal nuclei in rare abdominal hair follicles of wild type (FIG. 13A) but not mutant mice (FIG. 13B). Monoclonal antibodies to cytokeratin 18 and chromogranin A identify Merkel cells in both wild type (FIGS. 13C and 13E) and mutant (FIGS. 13D and 13F) mice. The original magnification was 100×.

FIGS. 14A through 14G show Math1 rescues the lack of chordotonal neurons in Drosophila ato mutant embryos. FIG. 14A shows a dorsal view of the thorax of a wild-type fly. Note there are regular array of bristles or macrochaetae. FIG. 14B shows a similar view of a transgenic fly in which Math1 was overexpressed using the UAS/GAL4 system (Brand and Perrimon, 1993). This ectopic expression leads to numerous extra bristles that are external sensory organs (another type of mechano receptor), not CHOs. Ectopic CHOs were produced in many other regions. FIG. 14C shows a lateral view of two abdominal clusters containing 6 CHOs in addition to external sensory organs, revealed by a neuronal-specific antibody (Mab 22C10). The 5 lateral CHOs form a cluster, and the sixth is dorsal to the cluster. FIG. 14D shows a similar view of an ato mutant embryo showing lack of the CHOs. FIG. 14E demonstrates ubiquitous expression of Math1 induces new CHO neurons in ato mutant embryos in the proper location. FIG. 14F shows in situ hybridization of whole mount third instar brain using the ato cDNA as a probe. Note expression in the developing optic lobes (“horse shoe” expression patterns) and two punctate clusters of cells in the middle of the brain lobes (arrow heads). FIG. 14G shows Math1 expression in Drosophila induces CHO formation in normal and ectopic locations. The (+) indicates presence of CHOs and (−) indicates their absence. Number of (+) in the first column is used to quantify the relative increase in the number of CHOs observed when Math1 is expressed.

FIG. 15 shows Math1/LacZ expression detected by X-gal staining. Math1/LacZ expression in E18.5 intestines (A to D). Cross section of Math1^{β-Gal/+} ileum (A) and colon (C), Math1^{β-Gal/-} ileum (B)and colon (D); Arrows indicate sparse lacZ-positive cells in heterozygous animals. Math1/LacZ expression in 5-month-old Math1^{β-Gal/+} mice (E and F). Longitudinal section of jejunum (E) and colon (F). Original magnification, ×400.

FIG. 16 illustrates Math1/lacZ expression detected by X-Gal staining. Longitudinal section of duodenum (A) and ileum (B) from five-month old Math1^{β-Gal/+} mice. Original magnification 400×.

FIG. 17 demonstrates a lack of goblet and enteroendocrine cells in E18.5 intestines. H&E staining reveals several goblet cells in wild-type duodenum [arrowheads in (A)] and non in null mutant (B); Alcian blue staining shows positively stained goblet cells in wild-type ileum [(arrowheads in (C)] but none in Math1^{β-Gal/-} null ileum (D). Serotonin-positive enteroendocrine cells [red-stained cells in (E), the arrow points to the cell enlarged in the inset] are evident in wild-type (E) but not Math1^{β-Gal/-} (F) jejunum. Original magnification, ×200.

FIG. 18 shows electron microscopy, cryptdin RT-PCR and colocalization of Math1/LacZ and proliferation marker Ki-67.(A) EM of the ileum reveals goblet cells (G)and enteroendocrine cells (E) in wild-type mice; neither of these secretory cells is formed in the Math1 null mice (B). Enterocytes [arrowheads in (A)and (B)] appear normal in Math1 null mice (B). Cryptdin mRNA (C) was detected in wild-type duodenum, jejunum, and ileum, but not in colon, whereas the Math1 null mutant lacked cryptdin RNA in all intestinal tissues examined. G6PDH mRNA level was used as a control. X-gal and Ki-67 antibody staining (blue cytoplasmic and red nuclear, respectively) in sections from adult duodenum (D) and ileum (E) (no hematoxylin counterstaining was applied). Paneth cells with apical granules, located at the bottom of crypts (arrow), show no Ki-67 staining. A subset of Ki-67-positive cells are also Math1/LacZ -positive (arrowheads). Original magnification, ×2500 (A and B), ×1000 (D and E).

FIG. 19 shows alkaline phosphatase and lactase activity of enterocytes in E18.5 intestine. The dark purple staining of the enterocyte brush border indicates that alkaline phosphatase activity (arrows) is similar in wild-type (A) and Math1 null (B) intestines. Arrowheads in the lower panels indicate that lactase activity (the dark blue line) is also similar in wild type (C) and null (D) intestines. The arrow in (D) indicates clustered b-galactosidase activity in the intervillus region of Math1 null intestine. Original magnification 400×.

FIG. 20 demonstrates colocalization of Math1/LacZ and proliferation marker Ki-67. X-Gal and Ki-67 immunostaining in hematoxylin counter-stained sections from duodenum (A) and ileum (B) from a five-month-old Math1^{β-Gal/+} mouse. Several cells in the crypts are Ki-67-positive (brown nuclear staining); a subset of these cells also express Math1/lacZ (blue cytoplasmic staining, arrows). Insets provide enlarged views of the boxed areas. Original magnification 400×.

FIG. 21 shows expression of Notch components and model for epithelial cell lineage differentiation in mouse intestine. (A) E18.5 small intestines were subjected to RT-PCR using primers specific to the indicated genes. G6PDH mRNA level served as a control. (B) Math1 is essential for secretory cells. Abbreviations: Sec, secretin; L, glucagons/peptide YY; CCK, cholecystokinin; SP, substance P; 5HT, serotonin; Som, somatostatin; GIP, gastric inhibitory peptide; Gas, gastrin.

FIG. 22 illustrates Hes-1 immunohistochemistry of E18.5 intestine. Jejunum sections from E18.5 embryos were subjected to Hes-1 antibody staining (no hematoxylin counterstaining was applied). The enterocytes are positive for Hes-1 (red nuclear staining, indicated by arrowheads) in wild type (A) and Math1 null (B) mice. The dark apical patches [arrow in (A)] represent nonspecific staining of the secretory products of the goblet cells. Original magnification 400×.

### DETAILED DESCRIPTION OF THE INVENTION

It is readily apparent to one skilled in the art that various embodiments and modifications can be made to the invention disclosed in this Application without departing from the scope and spirit of the invention.

I. Definitions

The term “a” or “an” as used herein in the specification may mean one or more. As used herein in the claim(s), when used in conjunction with the word “comprising”, the words “a” or “an” may mean one or more than one. As used herein “another” may mean at least a second or more.

The term “abnormal proliferation” as used herein is defined as any proliferation of any type of cell, wherein said cell is not under the constraints of normal cell cycle progression and wherein said proliferation can result in a tumor or any cancerous development.

The term “alteration” as used herein is defined as any type of change or modification to a nucleic acid or amino acid. Said change or modification includes any mutation, deletion, rearrangement, addition to a nucleic acid. This includes posttranscriptional processing such as addition of a 5 cap, intron processing and polyadenylation. Mutations can be nonsense, missense, frameshift, or could lead to a truncated amino acid or could alter the conformation of the amino acid. The alteration to a nucleic acid can be present in regulatory sequences or can affect trans-acting factors. Also, multiple alterations can be present. Said change or modification also includes any change to an amino acid including methylation, myristilation, acetylation, glycosylation, or a change to signals associated with processing of said amino acid including intracellular or intercellular localization signals and cleaving of extraneous amino acids. Said alteration can also affect degradation or folding of said protein.

The term “atonal-associated” as used herein is defined as any nucleic acid sequence or amino acid sequence which is the Drosophila atonal nucleic acid sequence or amino acid sequence, or is any sequence which is homologous to or has significant sequence similarity to said nucleic acid or amino acid sequence, respectively. The sequence can be present in any animal including mammals and insects. As used herein, significant sequence similarity means similarity is greater than 25% and can occur in any region of another sequence. Examples of atonal-associated include but are not limited to Math1 (mouse atonal homolog 1), Cath1 (chicken atonal homolog 1), Hath1 (human atonal homolog 1), and Xath1 (Xenopus atonal homolog 1). Furthermore, multiple homologous or similar sequences can exist in an animal.

The term “defect” as used herein is defined as an alteration, mutation, flaw or loss of expression of an atonal-associated sequence. A skilled artisan is aware that loss of expression concerns expression levels of an atonal-associated sequence which are not significant or detectable by standard means in the art. A skilled artisan is also aware that loss, or absence, of expression levels in an adult organism, such as a human, occurs naturally and leads to impairment of hearing over time. Thus, “defect” as used herein includes the natural reduction or loss of expression of an atonal-associated sequence.

The term “delivering” as used herein is defined as bringing to a destination and includes administering, as for a therapeutic purpose.

The term “delivery vehicle” as used herein is defined as an entity which is associated with transfer of another entity. Said delivery vehicle is selected from the group consisting of an adenoviral vector, a retroviral vector, an adeno-associated vector, a plasmid, a liposome, a nucleic acid, a peptide, a lipid, a carbohydrate and a combination thereof.

The term “detectable condition” as used herein is defined as any state of health or status of an animal, organ or tissue characterized by specific developmental or pathological symptoms. Examples include but are not limited to loss of hair cells, cerebellar granule neuron deficiencies, hearing impairment, imbalance, joint disease, osteoarthritis, abnormal proliferation of neoplastic neuroectodernal cells and formation of medulloblastoma.

The term “downregulated expression” as used herein is defined as the expression of an atonal-associated sequence in approximately less than wild type quantities in a cell, including substantially lacking any expression, in which the atonal-associated sequence is not naturally found in the cell.

The term “gastrointestinal condition” as used herein is defined as a condition or disease which affects at least one aspect of the gastrointestinal system of an individual, including the small intestine and large intestine (colon). The gastrointestinal condition may be the direct result of a disease, for example, although it may be an indirect result of a disease. Examples include damage to the intestine as a result of a disease or infection. Examples of gastrointestinal diseases include inflammatory bowel disease, irritable bowel syndrome, necrotizing entercolitis, cancer, and pathogenic infection.

The term “heterologous” as used herein is defined as nucleic acid sequence which is of or relating to nucleic acid sequence not naturally occurring in a particular locus. In an alternative embodiment, the heterologous nucleic acid sequence naturally occurs in a particular locus, but contains a molecular alteration compared to the naturally occurring locus. For instance, a wild-type locus of an atonal-associated sequence can be used to replace a defective copy of the same sequence.

The term “inactivated” as used herein is defined as a state in which expression of a nucleic acid sequence is reduced or completely eliminated. Said inactivation can occur by transfer or insertion of another nucleic acid sequence or by any means standard in the art to affect expression levels of a nucleic acid sequence.

The term “precursors” as used herein is defined as progenitor cells from which other cells derive their origin and/or properties.

The term “regulatable reporter sequence” as used herein is defined as any sequence which directs transcription of another sequence and which itself is under regulatory control by an extrinsic factor or state. Examples of extrinsic factors or states include but are not limited to exposure to chemicals, nucleic acids, proteins, peptides, lipids, carbohydrates, sugars, light, sound, hormones, touch, or tissue-specific milieu. Examples of regulatable reporter sequences include the GAL promoter sequence and the tetracycline promoter/transactivator sequence.

The term “regulatory sequence” as used herein is defined as any sequence which controls either directly or indirectly the transcription of another sequence. Said control can be either regarding the initiation or cessation of transcription or regarding quantity or tissue distribution of transcription.

The term “reporter sequence” as used herein is defined as any sequence which demonstrates expression by a regulatory sequence. Said reporter sequence can be used as a marker in the form of an RNA or in a protein. Examples of reporter sequences are b-galactosidase, green fluorescent protein (GFP), blue fluorescent protein (BFP), neomycin, kanamycin, luciferase, b-glucuronidase and chloramphenicol transferase (CAT). In a specific aspect of the present invention, the presence and quantity of the reporter sequence product, whether it be a nucleic acid or amino acid, reflects the level of transcription by the promoter sequence which regulates it.

The term “therapeutically effective” as used herein is defined as the amount of a compound required to improve some symptom associated with a disease. For example, in the treatment of hearing impairment, a compound which improves hearing to any degree or arrests any symptom of hearing impairment would be therapeutically effective. In the treatment of a joint disease, a compound which improves the health or movement of a joint to any degree or arrests any symptom of a joint disease would be therapeutically effective. In the treatment of abnormal proliferation of cells, a compound which reduces the proliferation would be therapeutically effective. In the treatment of cancer, a compound which reduces proliferation of the cells, reduces tumor size, reduces metastases, reduces proliferation of blood vessels to said cancer, facilitates an immune response against the cancer would be therapeutically effective, for example. A therapeutically effective amount of a compound is not required to cure a disease but will provide a treatment for a disease.

The term “upregulated expression” as used herein is defined as the expression of an atonal-associated sequence in approximately wild type or greater than wild type quantities in a cell in which the atonal-associated sequence is naturally found.

The term “vector” as used herein is defined as a biological vehicle for delivery of a specific entity. In a specific embodiment the entity is an atonal-associated nucleic acid.

II. The Present Invention

The mouse small intestinal epithelium consists of four principal cell types deriving from one multipotent stem cell: enterocytes, goblet, enteroendocrine, and Paneth cells. Previous studies showed that Math1, a basic helix-loop-helix (bHLH) transcription factor, is expressed in the gut. However, the present invention demonstrates that, although Math1 is involved in governing differentiation in neuronal cells, it was surprising that Math1 has no detectable levels in the gut nervous system. The present invention demonstrates that loss of Math1 leads to depletion of goblet cells (which secrete mucous important for food movement), enteroendocrine cells (which secrete regulatory peptides), and Paneth cells (which secrete microbe-fighting peptides) without affecting enterocytes. Colocalization of Math1 with Ki-67 in some proliferating cells suggests that secretory cells (goblet, enteroendocrine, and Paneth cells) arise from a common progenitor that expresses Math1, whereas absorptive cells (enterocytes, which absorb nutrients) arise from a progenitor that is Math1-independent. The continuous, rapid renewal of these cells makes the intestinal epithelium a model system for the study of stem cell regeneration and lineage commitment. Furthermore, the MATH1 protein regulates the Delta-Notch signaling pathway that governs endocrine cell differentiation. Clearly, a basic understanding of the intestinal cell differentiation will provide new treatments for such diseases as irritable bowel syndrome and other abnormalities in motility of the gut. Also, since these intestinal cells depend on these regulatory pathways to signal them to stop proliferating, these pathways are useful for addressing the mechanisms of colon cancers. Furthermore, understanding the regulatory control of intestinal stem cells could lead to treatments to regenerate damaged intestinal tissue. In a specific embodiment, a dormant stem cell is provided to an individual with at least one regulatory factor to encourage differentiation and replace cells lost to injury. In a specific embodiment, the regulatory factor for enhancing differentiation of an intestinal stem cell to a secretory intestinal cell is a bone morphogenetic protein (BMP), such as GDF7, a BMP known to induce Math1 in spinal cord. In another specific embodiment, the regulatory factor for enhancing differentiation of an intestinal stem cell to an absorptive intestinal cell is a member of the hairy/enhancer of split (HES) family. In a specific embodiment, homologs of Hes family members downregulate atonal-related sequences. Examples of Hes family members include Hes1, Hes2, Hes3, Hes4, Hes5, Hes6, Hes7, and Hes/ESp1 related proteins (HERP1 and HERP2) (which are upregulated by Notch and can heterodimerize with Hes members to repress bHLH gene expression).

In a specific embodiment, similar methods and compositions are utilized for other types of stem cells.

In one aspect of the present invention there are methods and reagents which include utilization of an atonal-associated nucleic acid or amino acid sequence for the therapeutic use of a gastrointestinal. Thus, any homolog or ortholog of atonal (from Drosophila) including but not limited to Cath1 (from chicken), Hath1 (from human), Math1 (from mice) or Xath1 (from Xenopus) can be used in the present invention. In a preferred embodiment these sequences are directed to treatment of an animal, specifically a human, for a gastrointestinal condition. It is within the scope of the invention to encompass any sequence which is homologous to or has significant sequence similarity to said nucleic acid or amino acid sequence, respectively. The sequence can be present in any animal including mammals and insects. As used herein, significant sequence similarity means similarity (identity of amino acid residues or nucleic acid bases) is greater than 25% and can occur in any region of the sequence. In another embodiment an atonal-associated sequence as used herein has greater than about 50% sequence similarity, greater than about 70% similarity, or greater than about 80% similarity.

It is within the scope of the present invention that an atonal-associated nucleic acid sequence or amino acid sequence is utilized wherein domains important for activity, such as the basic HLH region, are included in a molecule but further comprise alterations, mutations, deletions or substitutions in regions of the nucleic acid or amino acid sequence which are not part of a domain important for an activity and do not affect its function.

Examples of atonal-associated sequences include but are not limited to Math1 (mouse atonal homolog 1), Cath1 (chicken atonal homolog 1), Hath1 (human atonal homolog 1), and Xath1 (Xenopus atonal homolog 1). Such examples are represented in SEQ ID NO:1 through SEQ ID NO:66, although others very likely exist in related organisms. A skilled artisan is cognizant of means to identify such sequences which have significant similarity, such as searching database collections of nucleic and amino acid sequence located on the World Wide Web, including at the site for the National Center for Biotechnology Information's GenBank database.

The sequences provided herein and the corresponding GenBank Accession numbers are listed parenthetically as follows: SEQ ID NO:1 (NM_{—}005172); SEQ ID NO:2 (NP_{—}005163.1); SEQ ID NO:3 (AW413228); SEQ ID NO: 4 (NM_{—}009719); SEQ ID NO:5 (NP_{—}033849.1); SEQ ID NO:6 (NM_{—}009718); SEQ ID NO: 7 (NP_{—}033848.1) SEQ ID NO:8 (NM_{—}009717); SEQ ID NO: 9 (NP_{—}033847.1); SEQ ID NO:10 (NM_{—}007500); SEQ ID NO: 11(NP_{—}031526.1); SEQ ID NO:12 (NM_{—}007501); SEQ ID NO:13 (AW280518); SEQ ID NO:14(AW236965); SEQ ID NO:15(AW163683); SEQ ID NO:16 (AF134869); SEQ ID NO: 17(AAD31451.1); SEQ ID NO:18 (AJ012660); SEQ ID NO:19 (CAA10106.1); SEQ ID NO:20 (AJ012659); SEQ ID NO:21 (CAA10105.1); SEQ ID NO:22 (AF071223); SEQ ID NO:23 (AAC68868.1); SEQ ID NO:24 (U76208); SEQ ID NO:25 (AAC53029.1); SEQ ID NO:26 (U76210); SEQ ID NO:27 (AAC53033.1); SEQ ID NO:28 (U76209); SEQ ID NO:29 (AAC53032.1); SEQ ID NO:30 (U76207); SEQ ID NO:31 (AAC53028.1); SEQ ID NO:32 (AF036257); SEQ ID NO:33 (AAC15969.1); SEQ ID NO:34 (AF034778); SEQ ID NO:35 (AJ001178); SEQ ID NO:36 (CAA04572.1); SEQ ID NO:37 (Y07621); SEQ ID NO:38 (CAA68900.1); SEQ ID NO:39 (AF024536); SEQ ID NO:40 (AAB82272.1); SEQ ID NO:41 (D85188); SEQ ID NO:42 (BAA12738.1); SEQ ID NO:43 (D44480); SEQ ID NO:44(BAA07923.1); SEQ ID NO:45 (D43694); SEQ ID NO:46 (BAA07791.1); SEQ ID NO:47 (D85845); SEQ ID NO:48 (BAA12880.1); SEQ ID NO:49 (U93171); SEQ ID NO:50 (AAB58669.1); SEQ ID NO:51 (U93170); SEQ ID NO:52 (AAB58668.1); SEQ ID NO:53 (U61152); SEQ ID NO:54 (AAB41307.1); SEQ ID NO:55 (U61151); SEQ ID NO:56 (AAB41306.1); SEQ ID NO:57 (U61148); SEQ ID NO:58 (AAB41305.1); SEQ ID NO:59 (U61149); SEQ ID NO:60 (AAB41304.1); SEQ ID NO:61 (U61150); SEQ ID NO:62 (AAB41303.1); SEQ ID NO:63 (L36646); and SEQ ID NO:64 (AAA21879.1).

In an aspect of the invention there is an animal having a heterologous nucleic acid sequence replacing an allele of an atonal-associated nucleic acid sequence under conditions wherein said heterologous sequence inactivates said allele. In an alternative embodiment a heterologous sequence is delivered to a cell for extrachromosomal propagation. In another alternative embodiment a heterologous sequence is integrated into the chromosome of a cell in a locus other than the locus of an atonal-associated nucleic acid sequence. In a preferred embodiment said heterologous sequence is expressed under control of an atonal-associated regulatory sequence. In a specific embodiment both atonal-associated alleles are replaced. In an additional specific embodiment both atonal-associated alleles are replaced with nonidentical heterologous nucleic acid sequences. Methods to generate transgenic animals are well known in the art, and a skilled artisan would refer to such references as Transgenic Animals by Grosveld and Kollias (eds.) or Mouse Genetics and Transgenics: A Practical Approach by Jackson et al. (eds.).

In another embodiment of the present invention is a method for screening for a compound in an animal, wherein said compound affects expression of an atonal-associated nucleic acid sequence comprising delivering said compound to said animal wherein said animal has at least one allele of an atonal-associated nucleic acid sequence inactivated by insertion of a heterologous nucleic acid sequence wherein said heterologous nucleic acid sequence is under control of an atonal-associated regulatory sequence, and monitoring for a change in said expression of said atonal-associated nucleic acid sequence. Examples of regulatory sequences can include promoter sequences, enhancers or silencers.

In specific embodiments there is a compound which upregulates or downregulates said expression of an atonal-associated nucleic acid sequence. The upregulation or downregulation can be by increasing the rate of transcription or decreasing the rate of mRNA decay.

Another embodiment of the present invention is a compound which affects expression of an atonal-associated nucleic acid sequence. In specific embodiments said compound upregulates or downregulates expression of an atonal-associated nucleic acid sequence. In a specific embodiment said compound affects a gastrointestinal condition.

Another embodiment of the present invention is a method for screening for a compound in an animal, wherein the compound affects a detectable condition in the animal, comprising delivering the compound to the animal wherein at least one allele of an atonal-associated nucleic acid sequence in said animal is inactivated by insertion of a heterologous nucleic acid sequence, wherein said heterologous nucleic acid sequence is under the control of an atonal-associated regulatory sequence, and monitoring said animal for a change in the detectable condition. In another embodiment said delivery of said compound affects expression of said heterologous nucleic acid sequence. In specific embodiments said expression of said heterologous nucleic acid sequence is upregulated or downregulated. In additional specific embodiments the animal is a mouse, Drosophila, frog, zebrafish, rat, hamster and guinea pig.

Another embodiment of the present invention is a compound wherein said compound affects a gastrointestinal condition in a transgenic animal of the present invention. In specific embodiments said compound affects expression of a heterologous nucleic acid sequence. In additional specific embodiments said compound upregulates or downregulates expression of a heterologous nucleic acid sequence.

In other embodiments of the present invention are methods of treating an animal, including a human, for a gastrointestinal condition. Said methods include administering a therapeutically effective amount of an atonal-associated nucleic acid or amino acid sequence. In specific embodiments said administration is by a vector selected from the group consisting of a viral vector (including bacteriophage, animal and plant viruses), a plasmid, cosmid or any other nucleic acid based vector, a liposome, a nucleic acid, a peptide, a lipid, a carbohydrate and a combination thereof of said vectors. In a specific embodiment said viral vector is an adenovirus vector, a retrovirus vector, or an adeno-associated vector, including a lentivirus vector, Herpes virus vector, alpha virus vector, etc. Thus, a vector can be viral or non-viral. In another specific embodiment said vector is a cell. In a preferred embodiment said vector is an adenovirus vector comprising a cytomegalovirus IE promoter sequence and a SV40 early polyadenylation signal sequence. In another specific embodiment said cell is a human cell.

In an embodiment of the present invention there is provided a method for treating an organism for a disease that is a result of loss of functional atonal-associated nucleic acid or amino acid sequence. A skilled artisan is aware that this loss can be due to natural reduction or absence of significant (or to detectable levels) expression which occurs in an adult human.

In a specific embodiment, the present invention also provides a method of treating an animal in need of treatment for a deficiency in the intestine. This method comprises delivering a transcription factor having an amino acid with at least about 70% identity, preferably at least about 80% identity, and more preferably at least about 90% identity to the sequence AANARERRRMHGLNHAFDQLR to a cell in the animal. In some embodiments, the cell in the animal is located in the inner ear of the animal. Preferably, the transcription factor competes with atonal for binding to Daughterless protein (Jarman et al., 1993) or competes for binding with Math-1 to E47 protein (Akazawa et al., 1995).

In a preferred embodiment of the present invention there are compositions to treat an organism for various medical conditions, discussed herein, comprising an atonal-associated nucleic acid sequence or amino acid sequence in combination with a delivery vehicle, wherein said organism comprises a defect in an atonal-associated nucleic acid sequence. A skilled artisan is aware that an adult organism, such as an adult human, naturally does not express atonal to significant or detectable levels, but instead expresses atonal in an embryonic stage of development (see the Examples). Thus, in a preferred embodiment, compositions to treat an organism as discussed herein, include compositions to treat organisms who do not contain a mutation in an atonal nucleic acid or amino acid sequence but who naturally have atonal no longer expressed to significant or detectable levels.

In another embodiment of the present invention is a composition comprising an atonal-associated amino acid sequence or nucleic acid sequence in combination with a delivery vehicle wherein said vehicle delivers a therapeutically effective amount of an atonal-associated nucleic acid sequence or amino acid sequence into a cell. In specific embodiments said vehicle is the receptor-binding domain of a bacterial toxin or any fusion molecule or is a protein transduction domain. In a specific embodiment said protein transduction domain is from the HIV TAT peptide.

In another embodiment of the present invention there is a composition to treat an organism for cancer, wherein said organism comprises a defect in an atonal-associated nucleic acid sequence. In a specific embodiment the defect is a mutation or alteration of said atonal-associated nucleic acid sequence. In another specific embodiment the defect affects a regulatory sequence of said atonal-associated nucleic acid sequence. In an additional embodiment of the present invention there is a composition to treat an organism for cancer, wherein said organism comprises defect in a nucleic acid sequence which is associated with regulation of an atonal-associated nucleic acid sequence. In an additional embodiment of the present invention there is a composition to treat an organism for cancer, wherein said organism comprises a defect in an amino acid sequence which is associated with regulation of an atonal-associated nucleic acid sequence. In a specific embodiment said cancer is medulloblastoma.

III. Gastrointestinal Conditions

In specific embodiments, the methods of treatment for a gastrointestinal condition described herein are used alone or in conjunction with standard therapies for the gastrointestinal condition.

A. Inflammatory Bowel Disease

Inflammatory bowel disease is the name of a group of disorders that cause the intestines to become inflamed (red and swollen). The inflammation lasts a long time and usually is recurring. Symptoms include abdominal cramps and pain, diarrhea, weight loss and/or bleeding from the intestines. Two kinds of inflammatory bowel disease include Crohn's disease and ulcerative colitis. Crohn's disease usually causes ulcers (open sores) along the length of the small and large intestines. Crohn's disease either does not affect the rectum or causes inflammation or infection with drainage around the rectum. Ulcerative colitis usually causes ulcers in the lower part of the large intestine, often starting at the rectum.

Treatment for inflammatory bowel disease includes removing the inflammation by taking anti-inflammatory medicines including sulfasalazine (brand name: Azulfidine), olsalazine (brand name: Dipentum) and mesalamine (brand names: Asacol, Pentasa, Rowasa). An antibiotic, such as metronidazole (brand name: Flagyl), may be helpful for killing germs in the intestines, especially for Crohn's disease. Corticosteroids, such as prednisone, often are also prescribed.

B. Irritable Bowel Syndrome

Irritable bowel syndrome (IBS), also referred to as functional bowel syndrome, irritable colon, spastic bowel or spastic colon, is a problem with the intestines, wherein they squeeze too hard or not hard enough, causing food to move too quickly or too slowly through the intestines. Symptoms include bloating and gas, constipation, diarrhea, especially after eating or first thing in the morning, feeling an urge to have a bowel movement after already having had one, feeling a strong urge to have a bowel movement, and/or abdominal pain and cramping that may go away after having a bowel movement.

C. Pathogenic Organisms

1. Helicobacteriosis

Helicobacteriosis refers to infection of the gastrointestinal tract with the bacteria, Helicobacter pylori (H. pylori). It is a primary cause of ulcer disease and has revolutionized the treatment of peptic ulcer disease. It is also believed to be a cause of various cancers of the stomach.

H. pylon is a gram-negative spiral shaped organism that contains flagella (tail-like structure) and other properties that allow it to survive in the acidic environment of the stomach. In addition to flagella, which allow the organism to move around in the liquid mucous layer of the stomach, H. pylori also produces the enzyme “urease” that protects it from harm by gastric acid. As the production of this enzyme is relatively unusual, new diagnostic tests have enabled rapid identification of the bacteria. H. pylori also produces two other chemicals: the cytotoxin VacA and the protein CagA. Patients with ulcer disease are more likely to produce the cytotoxin (VacA). The CagA protein not only occurs frequently in ulcer disease but also in cancer. The bacteria is well adapted to survival within the stomach, surviving there for years. However, once infection begins, a form of chronic inflammation (chronic gastritis) always develops. In most individuals, initial infection causes little or no symptoms, although some individuals experience abdominal pain and nausea.

In about 15% of infected persons, ulcer disease develops either in the stomach or duodenum. Acid secretion increases in most patients with duodenal ulcers. This increase returns to normal once H. pylori is eliminated. It is now known that elimination of the bacteria will decrease substantially the risk of recurrent bouts of ulcer disease in the far majority (85% or so) of patients.

In the last decade it has been shown that H. pylon is not only the prime cause of ulcer disease of the stomach and duodenum, but is also strongly associated with various tumors of the stomach. Bacterial infection is 9 times more common in patients with cancer of the stomach, and 7 times more common in those with lymphoma of the stomach (tumor of the lymphatic tissue), called a MALT tumor. It is believed that the prolonged inflammation leads to changes in cell growth and tumors. Eliminating H. pylon can lead to regression of some tumors.

In addition to the above-described damage caused by H. pylon, some individuals lose normal gastric function, such as the ability to absorb vitamin B-12.

2. Giardiasis

Giardia is a microscopic parasite that can live in the human bowel, and infection by this parasite is called giardiasis. Some symptoms of giardiasis are diarrhea, belching, gas and cramps. Giardiasis is easy to contract upon drinking untreated spring water or stream water. Many animals carry Giardia in their feces and may introduce this parasite into rivers, streams and springs in rural areas.

IV. Nucleic Acid-Based Expression Systems

A. Vectors

One of skill in the art would be well equipped to construct a vector through standard recombinant techniques, which are described in Sambrook et al., 1989 and Ausubel et al., 1994, both incorporated herein by reference.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors can contain nucleic acid sequences that serve other functions as well and are described infra.

1. Promoters and Enhancers

A “promoter” is a control sequence that is a region of a nucleic acid sequence at which initiation and rate of transcription are controlled. It can contain genetic elements at which regulatory proteins and molecules can bind such as RNA polymerase and other transcription factors. A promoter may or may not be used in conjunction with an “enhancer,” which refers to a cis-acting regulatory sequence involved in the transcriptional activation of a nucleic acid sequence.

Naturally, it will be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. (1989), incorporated herein by reference. The promoters employed can be constitutive, tissue-specific, inducible, and/or useful under the appropriate conditions to direct high level expression of the introduced DNA segment, such as is advantageous in the large-scale production of recombinant proteins and/or peptides. The promoter can be heterologous or endogenous.

The identity of tissue-specific promoters or elements, as well as assays to characterize their activity, is well known to those of skill in the art. Examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), D1A dopamine receptor gene (Lee, et al., 1997), insulin-like growth factor II (Wu et al., 1997), human platelet endothelial cell adhesion molecule-1 (Almendro et al., 1996).

2. Initiation Signals and Internal Ribosome Binding Sites

A specific initiation signal also can be required for efficient translation of coding sequences. These signals include the ATG initiation codon or adjacent sequences. Exogenous translational control signals, including the ATG initiation codon, can need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression can be enhanced by the inclusion of appropriate transcription enhancer elements.

In certain embodiments of the invention, the use of internal ribosome entry sites (IRES) elements are used to create multigene, or polycistronic, messages. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

3. Multiple Cloning Sites

Vectors can include a multiple cloning site (MCS), which is a nucleic acid region that contains multiple restriction enzyme sites, any of which can be used in conjunction with standard recombinant technology to digest the vector. (See Carbonelli et al., 1999, Levenson et al., 1998, and Cocea, 1997, incorporated herein by reference.)

4. Splicing Sites

Most transcribed eukaryotic RNA molecules will undergo RNA splicing to remove introns from the primary transcripts. Vectors containing genomic eukaryotic sequences can require donor and/or acceptor splicing sites to ensure proper processing of the transcript for protein expression. (See Chandler et al., 1997, herein incorporated by reference.)

5. Polyadenylation Signals

In expression, one will typically include a polyadenylation signal to effect proper polyadenylation of the transcript. Specific embodiments include the SV40 polyadenylation signal and/or the bovine growth hormone polyadenylation signal, convenient and/or known to function well in various target cells.

6. Origins of Replication

In order to propagate a vector in a host cell, it can contain one or more origins of replication sites (often termed “ori”), which is a specific nucleic acid sequence at which replication is initiated.

7. Selectable and Screenable Markers

In certain embodiments of the invention, the cells contain nucleic acid construct of the present invention, a cell can be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP or enhanced GFP, whose basis is colorimetric analysis, are also contemplated. Alternatively, screenable enzymes such as herpes simplex virus thymidine kinase (tk) or chloramphenicol acetyltransferase (CAT) can be utilized. One of skill in the art would also know how to employ immunologic markers, possibly in conjunction with FACS analysis. Further examples of selectable and screenable markers are well known to one of skill in the art.

B. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®. Other examples of expression systems are well known in the art.

V. Nucleic Acid Detection

In addition to their use in directing the expression of atonal-associated proteins, polypeptides and/or peptides, the nucleic acid sequences disclosed herein have a variety of other uses. For example, they have utility as probes or primers or in any of the methods for embodiments involving nucleic acid hybridization, amplification of nucleic acid sequences, detection of nucleic acids, and other assays. A skilled artisan is aware of the following patents regarding details of these methods: U.S. Pat. Nos. 5,840,873; 5,843,640; 5,843,650; 5,843,651; 5,843,663; 5,846,708; 5,846,709; 5,846,717; 5,846,726; 5,846,729; 5,846,783; 5,849,481; 5,849,483; 5,849,486; 5,849,487; 5,849,497; 5,849,546; 5,849,547; 5,851,770; 5,851,772; 5,853,990; 5,853,993; 5,853,992; 5,856,092; 5,858,652; 5,861,244; 5,863,732; 5,863,753; 5,866,331; 5,866,336; 5,866,337; 5,900,481; 5,905,024; 5,910,407; 5,912,124; 5,912,145; 5,912,148; 5,916,776; 5,916,779; 5,919,626; 5,919,630; 5,922,574; 5,925,517; 5,925,525; 5,928,862; 5,928,869; 5,928,870; 5,928,905; 5,928,906; 5,929,227; 5,932,413; 5,932,451; 5,935,791; 5,935,825; 5,939,291; 5,942,391; European Application No. 320 308; European Application No. 329 822; GB Application No. 2 202 328; PCT Application No. PCT/US87/00880; PCT Application No. PCT/US89/01025; PCT Application WO 88/10315; PCT Application WO 89/06700; and PCT Application WO 90/07641.

VI. Kits

All of the essential materials and/or reagents required for detecting a sequence selected from SEQ ID NO:1 through SEQ ID NO:66 in a sample can be assembled together in a kit. This generally will comprise a probe or primers designed to hybridize specifically to individual nucleic acids of interest in the practice of the present invention, such as the nucleic acid sequences in SEQ ID NO:1 through SEQ ID NO:66. Also included can be enzymes suitable for amplifying nucleic acids, including various polymerases (reverse transcriptase, Taq, etc.), deoxynucleotides and buffers to provide the necessary reaction mixture for amplification. Such kits can also include enzymes and other reagents suitable for detection of specific nucleic acids or amplification products. Such kits generally will comprise, in suitable means, distinct containers for each individual reagent or enzyme as well as for each probe or primer pair.

VII. Atonal-Associated Nucleic Acids and Uses Thereof

A nucleic acid can be purified on polyacrylamide gels, cesium chloride centrifugation gradients, or by any other means known to one of ordinary skill in the art (see for example, Sambrook et al. 1989, incorporated herein by reference).

The term “nucleic acid” will generally refer to at least one molecule or strand of DNA, RNA or a derivative or mimic thereof, comprising at least one nucleobase, such as, for example, a naturally occurring purine or pyrimidine base found in DNA (e.g. adenine “A,” guanine “G,” thymine “T” and cytosine “C”) or RNA (e.g. A, G, uracil “U” and C). The term “nucleic acid” encompass the terms “oligonucleotide” and “polynucleotide.” The term “oligonucleotide” refers to at least one molecule of between about 3 and about 100 nucleobases in length. The term “polynucleotide” refers to at least one molecule of greater than about 100 nucleobases in length. These definitions generally refer to at least one single-stranded molecule, but in specific embodiments will also encompass at least one additional strand that is partially, substantially or fully complementary to the at least one single-stranded molecule. Thus, a nucleic acid can encompass at least one double-stranded molecule or at least one triple-stranded molecule that comprises one or more complementary strand(s) or “complement(s)” of a particular sequence comprising a strand of the molecule. As used herein, a single stranded nucleic acid can be denoted by the prefix “ss”, a double stranded nucleic acid by the prefix “ds”, and a triple stranded nucleic acid by the prefix “ts.”

Thus, the present invention also encompasses at least one nucleic acid that is complementary to a atonal-associated nucleic acid. In particular embodiments the invention encompasses at least one nucleic acid or nucleic acid segment complementary to the nucleic acid sequences set forth in SEQ ID NO:1 through SEQ ID NO:66, of those which are nucleic acid sequences. Nucleic acid(s) that are “complementary” or “complement(s)” are those that are capable of base-pairing according to the standard Watson-Crick, Hoogsteen or reverse Hoogsteen binding complementarity rules. As used herein, the term “complementary” or “complement(s)” also refers to nucleic acid(s) that are substantially complementary, as can be assessed by the same nucleotide comparison set forth above. The term “substantially complementary” refers to a nucleic acid comprising at least one sequence of consecutive nucleobases, or semiconsecutive nucleobases if one or more nucleobase moieties are not present in the molecule, are capable of hybridizing to at least one nucleic acid strand or duplex even if less than all nucleobases do not base pair with a counterpart nucleobase.

Herein certain embodiments, a “gene” refers to a nucleic acid that is transcribed. As used herein, a “gene segment” is a nucleic acid segment of a gene. In certain aspects, the gene includes regulatory sequences involved in transcription, or message production or composition. In particular embodiments, the gene comprises transcribed sequences that encode for a protein, polypeptide or peptide. In other particular aspects, the gene comprises an atonal-associated nucleic acid, and/or encodes an atonal-associated polypeptide or peptide coding sequences. In keeping with the terminology described herein, an “isolated gene” can comprise transcribed nucleic acid(s), regulatory sequences, coding sequences, or the like, isolated substantially away from other such sequences, such as other naturally occurring genes, regulatory sequences, polypeptide or peptide encoding sequences, etc. In this respect, the term “gene” is used for simplicity to refer to a nucleic acid comprising a nucleotide sequence that is transcribed, and the complement thereof. In particular aspects, the transcribed nucleotide sequence comprises at least one functional protein, polypeptide and/or peptide encoding unit. As will be understood by those in the art, this function term “gene” includes both genomic sequences, RNA or cDNA sequences or smaller engineered nucleic acid segments, including nucleic acid segments of a non-transcribed part of a gene, including but not limited to the non-transcribed promoter or enhancer regions of a gene. Smaller engineered gene nucleic acid segments can express, or can be adapted to express using nucleic acid manipulation technology, proteins, polypeptides, domains, peptides, fusion proteins, mutants and/or such like.

In certain embodiments, the nucleic acid sequence is a nucleic acid or nucleic acid segment. As used herein, the term “nucleic acid segment”, are smaller fragments of a nucleic acid, such as for non-limiting example, those that encode only part of the atonal-associated peptide or polypeptide sequence. Thus, a “nucleic acid segment” can comprise any part of the atonal-associated gene sequence(s), of from about 2 nucleotides to the full length of the atonal-associated peptide or polypeptide encoding region. In certain embodiments, the “nucleic acid segment” encompasses the full length atonal-associated gene(s) sequence. In particular embodiments, the nucleic acid comprises any part of the SEQ ID NO:1 through SEQ ID NO:66, of from about 2 nucleotides to the full length of the sequence disclosed in SEQ ID NO:1 through SEQ ID NO:66.

In certain embodiments, the nucleic acid segment can be a probe or primer. As used herein, a “probe” is a nucleic acid utilized for detection of another nucleic acid and is generally at least about 10 nucleotides in length. As used herein, a “primer” is a nucleic acid utilized for polymerization of another nucleic acid is generally at least about 10 nucleotides in length. A non-limiting example of this would be the creation of nucleic acid segments of various lengths and sequence composition for probes and primers based on the sequences disclosed in SEQ ID NO:1 through SEQ ID NO:66, of those which are nucleic acid sequences.

The nucleic acid(s) of the present invention, regardless of the length of the sequence itself, can be combined with other nucleic acid sequences, including but not limited to, promoters, enhancers, polyadenylation signals, restriction enzyme sites, multiple cloning sites, coding segments, and the like, to create one or more nucleic acid construct(s). As used herein, a “nucleic acid construct” is a recombinant molecule comprising at least two segments of different nucleic acid sequence. The overall length can vary considerably between nucleic acid constructs. Thus, a nucleic acid segment of almost any length can be employed, with the total length preferably being limited by the ease of preparation or use in the intended recombinant nucleic acid protocol.

In certain embodiments, the nucleic acid construct is a recombinant vector. As used herein, a “recombinant vector” is a nucleic acid comprising multiple segments of nucleic acids utilized as a vehicle for a nucleic acid sequence of interest. In certain aspects, the recombinant vector is an expression cassette. As used herein, an expression cassette is a segment of nucleic acid which comprises a gene of interest which can be transferred between different recombinant vectors by means well known in the art.

In particular embodiments, the invention concerns one or more recombinant vector(s) comprising nucleic acid sequences that encode an atonal-associated protein, polypeptide or peptide that includes within its amino acid sequence a contiguous amino acid sequence in accordance with, or essentially as set forth in, SEQ ID NO:2 through SEQ ID NO:66, of which sequences are amino acid sequences, corresponding to Homo sapiens or Mus musculus atonal-associated sequence. In other embodiments, the invention concerns recombinant vector(s) comprising nucleic acid sequences from other species that encode an atonal-associated protein, polypeptide or peptide that includes within its amino acid sequence a contiguous amino acid sequence in accordance with, or essentially as set forth in SEQ ID NO:2 through SEQ ID NO:66, of which sequences are amino acid sequences. In particular aspects, the recombinant vectors are DNA vectors.

It will also be understood that amino acid sequences or nucleic acid sequences can include additional residues, such as additional N- or C-terminal amino acids or 5′ or 3′ sequences, or various combinations thereof, and yet still be essentially as set forth in one of the sequences disclosed herein, so long as the sequence meets the criteria set forth above, including the maintenance of biological protein, polypeptide or peptide activity where expression of a proteinaceous composition is concerned. The addition of terminal sequences particularly applies to nucleic acid sequences that can, for example, include various non-coding sequences flanking either of the 5′ and/or 3′ portions of the coding region or can include various internal sequences, i.e., introns, which are known to occur within genes.

It will also be understood that this invention is not limited to the particular nucleic acid or amino acid sequences of SEQ ID NO: through SEQ ID NO:66, of which sequences are amino acids. Recombinant vectors and isolated nucleic acid segments can therefore variously include these coding regions themselves, coding regions bearing selected alterations or modifications in the basic coding region, and they can encode larger polypeptides or peptides that nevertheless include such coding regions or can encode biologically functional equivalent proteins, polypeptide or peptides that have variant amino acids sequences.

The nucleic acids of the present invention encompass biologically functional equivalent atonal-associated proteins, polypeptides, or peptides or atonal-associated proteins, polypeptides or polypeptides. Such sequences can arise as a consequence of codon redundancy or functional equivalency that are known to occur naturally within nucleic acid sequences or the proteins, polypeptides or peptides thus encoded. Alternatively, functionally equivalent proteins, polypeptides or peptides can be created via the application of recombinant DNA technology, in which changes in the protein, polypeptide or peptide structure can be engineered, based on considerations of the properties of the amino acids being exchanged. Changes designed by man can be introduced, for example, through the application of site-directed mutagenesis techniques as discussed herein below, e.g., to introduce improvements or alterations to the antigenicity of the protein, polypeptide or peptide, or to test mutants in order to examine atonal-associated protein, polypeptide or peptide activity at the molecular level.

Fusion proteins, polypeptides or peptides can be prepared, e.g., where the atonal associated coding regions are aligned within the same expression unit with other proteins, polypeptides or peptides having desired functions. Non-limiting examples of such desired functions of expression sequences include purification or immunodetection purposes for the added expression sequences, e.g., proteinaceous compositions that can be purified by affinity chromatography or the enzyme labeling of coding regions, respectively EP 266,032, or via deoxynucleotide H-phosphonate intermediates as described by Froehler et al., Nucl. Acids Res., 14:5399-5407, 1986,

As used herein an “organism” can be a prokaryote, eukaryote, virus and the like. As used herein the term “sequence” encompasses both the terms “nucleic acid” and “proteancecous” or “proteanaceous composition.” As used herein, the term “proteinaceous composition” encompasses the terms “protein”, “polypeptide” and “peptide.” As used herein “artificial sequence” refers to a sequence of a nucleic acid not derived from sequence naturally occurring at a genetic locus, as well as the sequence of any proteins, polypeptides or peptides encoded by such a nucleic acid. A “synthetic sequence”, refers to a nucleic acid or proteinaceous composition produced by chemical synthesis in vitro, rather than enzymatic production in vitro (i.e. an “enzymatically produced” sequence) or biological production in vivo (i.e. a “biologically produced” sequence).

VIII. Cancer Therapies

Given that the present invention is directed to methods and compositions for the treatment of abnormal cell proliferation, a discussion of therapies of cancer, which is the state of abnormal cell proliferation, is warranted. In a specific embodiment, a gastrointestinal condition is a cancer, and the cancer is treated with standard therapies in addition to treatments described herein.

A wide variety of cancer therapies, such as radiotherapy, surgery, chemotherapy and gene therapy, are known to one of skill in the art, can be used regarding the methods and compositions of the present invention.

A. Radiotherapeutic agents

Radiotherapeutic agents and factors include radiation and waves that induce DNA damage for example, g-irradiation, X-rays, UV-irradiation, microwaves, electronic emissions, radioisotopes, and the like. Therapy can be achieved by irradiating the localized tumor site with the above described forms of radiations.

Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

B. Surgery

Surgical treatment for removal of the cancerous growth is generally a standard procedure for the treatment of tumors and cancers. This attempts to remove the entire cancerous growth. However, surgery is generally combined with chemotherapy and/or radiotherapy to ensure the destruction of any remaining neoplastic or malignant cells. Thus, surgery can be used in the context of the present invention.

C. Chemotherapeutic Agents

These can be, for example, agents that directly cross-link DNA, agents that intercalate into DNA, or agents that lead to chromosomal and mitotic aberrations by affecting nucleic acid synthesis.

Agents that directly cross-link nucleic acids, specifically DNA, are envisaged and are shown herein, to eventuate DNA damage leading to a synergistic antineoplastic combination. Agents such as cisplatin, and other DNA alkylating agents can be used.

Agents that damage DNA also include compounds that interfere with DNA replication, mitosis, and chromosomal segregation. Examples of these compounds include adriamycin (also known as doxorubicin), VP-16 (also known as etoposide), verapamil, podophyllotoxin, and the like. Widely used in clinical setting for the treatment of neoplasms these compounds are administered through bolus injections intravenously at doses ranging from 25-75 mg/m2 at 21 day intervals for adriamycin, to 35-100 mg/m2 for etoposide intravenously or orally.

Cancer therapies also include a variety of combination therapies with both chemical and other types of treatments. Chemotherapeutics include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing.

D. Gene Therapy Administration

For gene therapy, a skilled artisan would be cognizant that the vector to be utilized must contain the gene of interest operatively linked to a promoter. For antisense gene therapy, the antisense sequence of the gene of interest would be operatively linked to a promoter. One skilled in the art recognizes that in certain instances other sequences such as a 3′ UTR regulatory sequences are useful in expressing the gene of interest. Where appropriate, the gene therapy vectors can be formulated into preparations in solid, semisolid, liquid or gaseous forms in the ways known in the art for their respective route of administration. Means known in the art can be utilized to prevent release and absorption of the composition until it reaches the target organ or to ensure timed-release of the composition. A pharmaceutically acceptable form should be employed which does not ineffectuate the compositions of the present invention. In pharmaceutical dosage forms, the compositions can be used alone or in appropriate association, as well as in combination, with other pharmaceutically active compounds. A sufficient amount of vector containing the therapeutic nucleic acid sequence must be administered to provide a pharmacologically effective dose of the gene product.

One skilled in the art recognizes that different methods of delivery can be utilized to administer a vector into a cell. Examples include: (1) methods utilizing physical means, such as electroporation (electricity), a gene gun (physical force) or applying large volumes of a liquid (pressure); and (2) methods wherein said vector is complexed to another entity, such as a liposome, viral vector or transporter molecule.

Accordingly, the present invention provides a method of transferring a therapeutic gene to a host, which comprises administering the vector of the present invention, preferably as part of a composition, using any of the aforementioned routes of administration or alternative routes known to those skilled in the art and appropriate for a particular application. Effective gene transfer of a vector to a host cell in accordance with the present invention to a host cell can be monitored in terms of a therapeutic effect (e.g. alleviation of some symptom associated with the particular disease being treated) or, further, by evidence of the transferred gene or expression of the gene within the host (e.g., using the polymerase chain reaction in conjunction with sequencing, Northern or Southern hybridizations, or transcription assays to detect the nucleic acid in host cells, or using immunoblot analysis, antibody-mediated detection, mRNA or protein half-life studies, or particularized assays to detect protein or polypeptide encoded by the transferred nucleic acid, or impacted in level or function due to such transfer).

These methods described herein are by no means all-inclusive, and further methods to suit the specific application will be apparent to the ordinary skilled artisan. Moreover, the effective amount of the compositions can be further approximated through analogy to compounds known to exert the desired effect.

Furthermore, the actual dose and schedule can vary depending on whether the compositions are administered in combination with other pharmaceutical compositions, or depending on interindividual differences in pharmacokinetics, drug disposition, and metabolism. Similarly, amounts can vary in in vitro applications depending on the particular cell line utilized (e.g., based on the number of vector receptors present on the cell surface, or the ability of the particular vector employed for gene transfer to replicate in that cell line). Furthermore, the amount of vector to be added per cell will likely vary with the length and stability of the therapeutic gene inserted in the vector, as well as also the nature of the sequence, and is particularly a parameter which needs to be determined empirically, and can be altered due to factors not inherent to the methods of the present invention (for instance, the cost associated with synthesis). One skilled in the art can easily make any necessary adjustments in accordance with the exigencies of the particular situation.

It is possible that cells containing the therapeutic gene can also contain a suicide gene (i.e., a gene which encodes a product that can be used to destroy the cell, such as herpes simplex virus thymidine kinase). In many gene therapy situations, it is desirable to be able to express a gene for therapeutic purposes in a host cell but also to have the capacity to destroy the host cell once the therapy is completed, becomes uncontrollable, or does not lead to a predictable or desirable result. Thus, expression of the therapeutic gene in a host cell can be driven by a promoter although the product of said suicide gene remains harmless in the absence of a prodrug. Once the therapy is complete or no longer desired or needed, administration of a prodrug causes the suicide gene product to become lethal to the cell. Examples of suicide gene/prodrug combinations which can be used are Herpes Simplex Virus-thymidine kinase (HSV-tk) and ganciclovir, acyclovir or FIAU; oxidoreductase and cycloheximide; cytosine deaminase and 5-fluorocytosine; thymidine kinase thymidilate kinase (Tdk::Tmk) and AZT; and deoxycytidine kinase and cytosine arabinoside.

The method of cell therapy can be employed by methods known in the art wherein a cultured cell containing a copy of a nucleic acid sequence or amino acid sequence of Math1 is introduced.

In yet another embodiment, the secondary treatment is a secondary gene therapy in which a second therapeutic polynucleotide is administered before, after, or at the same time a first therapeutic polynucleotide encoding all of part of an atonal-associated polypeptide. Delivery of a vector encoding either a full length or partial atonal-associated polypeptide in conjunction with a second vector encoding another gene product will have a combined anti-hyperproliferative effect on target tissues. Alternatively, a single vector encoding both genes can be used.

E. Immunotherapy

Immunotherapeutics, generally, rely on the use of immune effector cells and molecules to target and destroy cancer cells. The immune effector can be, for example, an antibody specific for some marker on the surface of a tumor cell. The antibody alone can serve as an effector of therapy or it can recruit other cells to actually effect cell killing. The antibody also can be conjugated to a drug or toxin (chemotherapeutic, radionuclide, ricin A chain, cholera toxin, pertussis toxin, etc.) and serve merely as a targeting agent. Alternatively, the effector can be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with Ad-atonal-associated gene therapy. The general approach for combined therapy is discussed below. Generally, the tumor cell must bear some marker that is amenable to targeting, i.e., is not present on the majority of other cells. Many tumor markers exist and any of these can be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

IX. Combination Treatments

It can be desirable in utilizing the present invention to combine the compositions with other agents effective in the treatment of hyperproliferative disease, such as anti-cancer agents. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process can involve contacting the cells with the expression construct and the agent(s) or multiple factor(s) at the same time. This can be achieved by contacting the cell with a single composition or pharmacological formulation that includes both agents, or by contacting the cell with two distinct compositions or formulations, at the same time, wherein one composition includes the expression construct and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with gene therapy. For example, the herpes simplex-thymidine kinase (HS-tK) gene, when delivered to brain tumors by a retroviral vector system, successfully induced susceptibility to the antiviral agent ganciclovir (Culver, et al., 1992). In the context of the present invention, it is contemplated that an atonal-associated gene therapy could be used similarly in conjunction with chemotherapeutic, radiotherapeutic, or immunotherapeutic intervention, in addition to other pro-apoptotic or cell cycle regulating agents.

Alternatively, the gene therapy can precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one can contact the cell with both modalities within about 12-24 h of each other and, more preferably, within about 6-12 h of each other. In some situations, it can be desirable to extend the time period for treatment significantly, however, where several d (2, 3, 4, 5, 6 or 7) to several wk (1, 2, 3, 4, 5, 6, 7 or 8) lapse between the respective administrations.

Various combinations can be employed, gene therapy is “A” and the secondary agent, such as radio- or chemotherapy, is “B”:

A/B/A B/A/B B/B/A A/A/B A/B/B B/A/A A/B/B/B B/A/B/B

B/B/B/A B/B/A/B A/A/B/B A/B/A/B A/B/B/A B/B/A/A

B/A/B/A B/A/A/B A/A/A/B B/A/A/A A/B/A/A A/A/B/A

Administration of the therapeutic expression constructs of the present invention to a patient will follow general protocols for the administration of chemotherapeutics, taking into account the toxicity, if any, of the vector. It is expected that the treatment cycles would be repeated as necessary. It also is contemplated that various standard therapies, as well as surgical intervention, can be applied in combination with the described hyperproliferative cell therapy.

A. Inhibitors of Cellular Proliferation

The tumor suppressor oncogenes function to inhibit excessive cellular proliferation. The inactivation of these genes destroys their inhibitory activity, resulting in unregulated proliferation. The tumor suppressors p53, p16 and C-CAM are specific embodiments utilized in the present invention. Other genes that can be employed according to the present invention include Rb, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, ab1, E1A, p300, genes involved in angiogenesis (e.g., VEGF, FGF, thrombospondin, BAI-1, GDAIF, or their receptors) and MCC.

B. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists. Different family members have been shown to either possess similar functions to Bcl-2 (e.g., BclXL, BclW, BclS, Mcl-1, A1, Bfl-1) or counteract Bcl-2 function and promote cell death (e.g., Bax, Bak, Bik, Bim, Bid, Bad, Harakiri).

C. Other agents

It is contemplated that other agents can be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers.

X. Dosage and Formulation

The amino acid sequences, nucleic acid sequences, stem cells and/or regulatory factors (all active ingredients) of this invention can be formulated and administered to treat a variety of disease states by any means that produces contact of the active ingredient with the agent's site of action in the body of an animal. They can be administered by any conventional means available for use in conjunction with pharmaceuticals, either as individual therapeutic active ingredients or in a combination of therapeutic active ingredients. They can be administered alone, or with a pharmaceutically acceptable carrier selected on the basis of the chosen route of administration and standard pharmaceutical practice.

The dosage administered will be a therapeutically effective amount of active ingredient and will, of course, vary depending upon known factors such as the pharmacodynamic characteristics of the particular active ingredient and its mode and route of administration; age, sex, health and weight of the recipient; nature and extent of symptoms; kind of concurrent treatment, frequency of treatment and the effect desired.

The active ingredient can be administered orally in solid dosage forms such as capsules, tablets and powders, or in liquid dosage forms such as elixirs, syrups, emulsions and suspensions. The active ingredient can also be formulated for administration parenterally by injection, rapid infusion, nasopharyngeal absorption or dermoabsorption. The agent can be administered intramuscularly, intravenously, or as a suppository. In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

Additionally, standard pharmaceutical methods can be employed to control the duration of action. These are well known in the art and include control release preparations and can include appropriate macromolecules, for example polymers, polyesters, polyamino acids, polyvinyl, pyrolidone, ethylenevinylacetate, methyl cellulose, carboxymethyl cellulose or protamine sulfate. The concentration of macromolecules as well as the methods of incorporation can be adjusted in order to control release. Additionally, the agent can be incorporated into particles of polymeric materials such as polyesters, polyamino acids, hydrogels, poly (lactic acid) or ethylenevinylacetate copolymers. In addition to being incorporated, these agents can also be used to trap the compound in microcapsules.

Useful pharmaceutical dosage forms for administration of the compounds of this invention can be illustrated as follows.

Capsules: Capsules are prepared by filling standard two-piece hard gelatin capsulates each with a therapeutically effective amount of powdered active ingredient, 175 milligrams of lactose, 24 milligrams of talc and 6 milligrams magnesium stearate.

Soft Gelatin Capsules: A mixture of active ingredient in soybean oil is prepared and injected by means of a positive displacement pump into gelatin to form soft gelatin capsules containing a therapeutically effective amount of the active ingredient. The capsules are then washed and dried.

Tablets: Tablets are prepared by conventional procedures so that the dosage unit is a therapeutically effective amount of active ingredient. 0.2 milligrams of colloidal silicon dioxide, 5 milligrams of magnesium stearate, 275 milligrams of microcrystalline cellulose, 11 milligrams of cornstarch and 98.8 milligrams of lactose. Appropriate coatings can be applied to increase palatability or to delay absorption.

Injectable: A parenteral composition suitable for administration by injection is prepared by stirring 1.5% by weight of active ingredients in 10% by volume propylene glycol and water. The solution is made isotonic with sodium chloride and sterilized.

Suspension: An aqueous suspension is prepared for oral administration so that each 5 millimeters contain a therapeutically effective amount of finely divided active ingredient, 200 milligrams of sodium carboxymethyl cellulose, 5 milligrams of sodium benzoate, 1.0 grams of sorbitol solution U.S.P. and 0.025 millimeters of vanillin.

Accordingly, the pharmaceutical composition of the present invention can be delivered via various routes and to various sites in an animal body to achieve a particular effect (see, e.g., Rosenfeld et al. (1991), supra; Rosenfeld et al., Clin. Res., 39(2), 311A (1991a); Jaffe et al., supra; Berkner, supra). One skilled in the art will recognize that although more than one route can be used for administration, a particular route can provide a more immediate and more effective reaction than another route. Local or systemic delivery can be accomplished by administration comprising application or instillation of the formulation into body cavities, inhalation or insufflation of an aerosol, or by parenteral introduction, comprising intramuscular, intravenous, peritoneal, subcutaneous, intradermal, as well as topical administration.

The composition of the present invention can be provided in unit dosage form wherein each dosage unit, e.g., a teaspoonful, tablet, solution, or suppository, contains a predetermined amount of the composition, alone or in appropriate combination with other active agents. The term “unit dosage form” as used herein refers to physically discrete units suitable as unitary dosages for human and animal subjects, each unit containing a predetermined quantity of the compositions of the present invention, alone or in combination with other active agents, calculated in an amount sufficient to produce the desired effect, in association with a pharmaceutically acceptable diluent, carrier, or vehicle, where appropriate. The specifications for the unit dosage forms of the present invention depend on the particular effect to be achieved and the particular pharmacodynamics associated with the pharmaceutical composition in the particular host.

These methods described herein are by no means all-inclusive, and further methods to suit the specific application will be apparent to the ordinary skilled artisan. Moreover, the effective amount of the compositions can be further approximated through analogy to compounds known to exert the desired effect.

The following examples are offered by way of example, and are not intended to limit the scope of the invention in any manner.

### EXAMPLE 1

### Mouse Atonal Homolog 1 (Math1)

It has been found that the present methods for the treatment of the hearing impaired have failed to address the problem directly, that is, the regeneration of auditory hair cell populations. The present invention in a preferred embodiment is directed to a member of the bHLH family, the Math1 gene or another atonal-associated nucleic acid sequence, and its requirement for generation of cerebellar granule neurons and inner ear hair cells. This discovery has wide ramifications not only for understanding neurodevelopment but also for therapies for a variety of prevalent disorders, as described below.

The mouse atonal homolog 1 (Math1) is expressed in the precursors of the cerebellar granule neurons; a few cells in the dorsal portion of the developing spinal cord; the inner ear; Merkel cells (touch receptors on the skins); and joints. Overexpressing Math1 in an otherwise differentiated cell can induce the formation or differentiation into a progenitor or mature inner ear hair cell-like cell.

Math1 expression in the precursors of the cerebellar granule neurons suggests it is required for function in the cerebellum and brain. The cerebellum is essential for fine motor coordination and posture, and its dysfunction disrupts balance, speech and limb movements. Cerebellar development typically begins at about embryonic day 9.5 (E9.5) when a small group of cells in the hindbrain proliferates and migrates rostrally to form the external granule layer, brain stem, and pontine neurons. This population of neuronal progenitors, which continues to express Math1, further proliferates and migrates internally to form the cerebellar granule neurons that are the predominant neuronal population in the cerebellum and brain. Mice that do not express Math1 completely lack cerebellar granule neurons and their precursors. Math1 is thus essential for the generation of these neurons and endows the very sparse population of neurons at E9.5 with the ability to proliferate into billions and then differentiate (Ben-Arie et al., 1997). Both these functions are of great medical significance. To understand normal proliferation provides necessary insight into abnormal proliferation, as observed in cancer. Cerebellar tumors of the primitive neuroectodermal type (e.g., medulloblastoma) are the most common solid malignancy in children. Math1 expressing cells contribute significantly to these tumors.

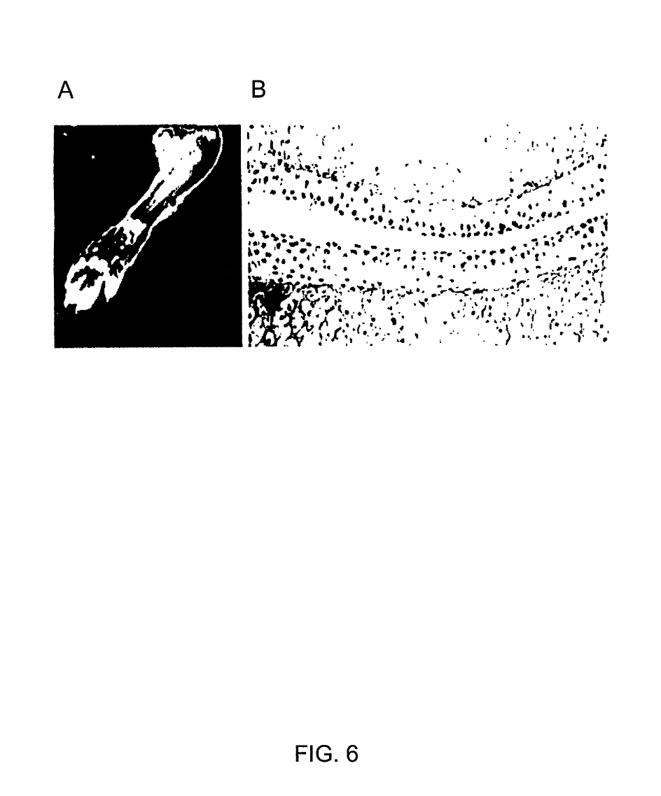

Math1 is expressed in the non-ossified joint cartilage (see FIG. 6) that typically degenerates in osteoarthritis. This is the most prevalent form of arthritis, with 90% of people over 40 showing some degree of osteoarthritis in one or more joints. Given the properties of Math1 in cellular generation and proliferation, its artificial expression in affected joints can allow regeneration of the cells that constitute non-ossified cartilage.

Disclosed herein are compositions and methods for the use of the Math1 gene, its human homolog (Hath1) or any of its homologs, orthologs, chimeric fusion proteins or derivatives of any suitable atonal-associated nucleic acid sequence or any another atonal-associated nucleic acid sequence. To learn about the functions of Math1 in mammals, the Math1 gene was deleted from a mouse using a strategy that permitted detection of cells that express Math1. Disclosed are the creation and characterization of mice that can be used to screen for compounds which could be utilized to decrease or augment Math1 expression in inner ear hair cells and other cells in which Math1 expression is associated.

Methods are also disclosed for the study, characterization and treatment of neoplastic proliferation of neuroectoderrnal origin since Math1 expression is essential for the generation and proliferation of cerebellar granule neurons. Also, it has been discovered that Math1 plays a role in the development of cells that produce non-ossified joint cartilage, which are associated with the development of osteoarthitis. These discoveries have led to a method of screening for compounds that can be helpful for the treatment of inner ear hair cell loss and other diseases that occur due to the functional loss of Math1, such as osteoarthritis.

More particularly, the present invention also provides an animal heterozygous for Math1 gene inactivation or an another atonal-associated nucleic acid sequence, wherein at least one Math1 allele or another atonal-associated nucleic acid sequence has been replaced by insertion of a heterologous nucleic acid sequence, wherein the inactivation of the Math1 or atonal-associated sequence prevents expression of the Math1 or atonal-associated allele. The mouse can be further used to generate mice homozygous for Math1 or another atonal-associated sequence gene inactivation and can further include a second heterologous nucleic acid sequence, wherein at least one of the heterologous genes is used to detect expression driven by the Math1 or atonal-associated sequence regulatory elements. The complete or partial inactivation of the functional Math1 or atonal-associated sequence can be detected in, e.g., proprioreceptory cells, granule neurons and their progenitor cells, or non-ossified cartilage cells.

Examples of heterologous nucleic acid sequences are reporter sequences such as b-galactosidase, green fluorescent protein (GFP), blue fluorescent protein (BFP), neomycin, kanamycin, luciferase, β-glucuronidase and chloramphenicol transferase (CAT). The Math1 or atonal-associated sequence can also be replaced under the control of regulatable promoter sequences or can be a tissue-specific promoter sequences. Said promoter sequences can be partial or can contain the entire promoter.

The present invention can also be used as, or as part of, a method for screening for a compound, wherein the administration of the compound affects a developmental and/or pathological gastrointestinal condition wherein said condition is a result of reduction in expression of the Math1 or atonal-associated sequence, the method including, administering the compound to a transgenic mouse that is homozygous for Math1 or atonal-associated sequence inactivation, wherein at least one Math1 or atonal-associated allele is inactivated by insertion of a heterologous nucleic acid sequence, wherein the inactivation of the Math1 or atonal-associated sequence prevents expression of the Math1 or atonal-associated gene, and monitoring the mouse for a change in the developmental and/or pathological gastrointestinal condition. As used herein, the screen provides for a compound that by upregulating expression of a heterologous nucleic acid sequence is a positive effector and for a compound that by downregulating expression of a heterologous nucleic acid sequence is a negative effector.

Yet another embodiment of the present invention is a method of promoting mechanoreceptive cell growth, that includes contacting a cell with a Math1 or atonal-associated protein or gene in an amount effective to cause said cell to express an inner ear hair cell marker. An example of a hair cell marker for use with the method is calretinin. The cell can be contacted with a vector that expresses a Math1 or atonal-associated nucleic acid sequence or amino acid sequence. Math1 or atonal-associated nucleic acid sequence-expressing recombinant vectors can include an adenoviral vector, a retroviral vector, an adeno-associated vector, a plasmid, a liposome, a protein, a lipid, a carbohydrate and a combination thereof of said vectors. Math1 or atonal-associated sequence can be under the control of, e.g., a cytomegalovirus IE promoter sequence or the cytomegalovirus IE promoter sequence and a SV40 early polyadenylation signal sequence, or any other combination of appropriate promoter sequences, enhancer sequence, and polyadenylation.

Furthermore, a method is disclosed for treating hearing impairment or an imbalance disorder that includes administering to an animal, including a human, with hearing loss or an imbalance disorder a therapeutically effective amount of a Math1 or atonal-associated amino acid sequence or nucleic acid sequence. The hearing or balance impairment can be complete or partial and can affect either one ear or both ears. In a preferred embodiment, there is a substantial impairment of hearing. Hearing and an imbalance disorder can be affected separately or concomitantly in an animal to be treated, and said hearing and/or an imbalance disorder could be as a result of trauma, disease, age-related condition, or could be due to loss of hair cells for any reason.

The present invention is also directed to a composition that includes a Math1 or atonal-associated protein or gene in combination with a delivery vehicle, wherein the delivery vehicle causes a therapeutically effective amount of Math1 or atonal-associated sequence to be delivered into a cell. The delivery vehicle can be further defined as a vector that comprises a Math1 or atonal-associated amino acid sequence or nucleic acid sequence in an animal cell. The vector can be a retroviral or an adenoviral vector or any other nucleic acid based vector, which can even be dispersed in a pharmacologically acceptable formulation, and used for intralesional administration. The composition can even be a partially or fully purified protein that is delivered using a liposome, a protein, a lipid or a carbohydrate that promotes the entry of a Math1 or atonal-associated protein into a cell. Examples of proteins that can be used as delivery vehicles include the receptor-binding domains (the non-catalytic regions) of bacterial toxins, such as, e.g., Exotoxin A, cholera toxin and Ricin toxin or protein transduction domains, such as from the HIV TAT protein (Schwarze et al., 1999) (see Example 22). The composition for delivering Math1 can be a fusion protein.

A skilled artisan is aware that methods to treat animals as disclosed in the invention can be either in utero or after birth. Treatment can be given to an embryo and can occur either ex vivo or in vivo.

### EXAMPLE 2

### Animal Model for Organogenesis

An effective animal model for deficiency in a gene that controls organogenesis will most often have both alleles stably inactivated so that, throughout embryogenesis, one or more tissues cannot revert to a functional wild-type allele. One method of generating animals with an altered genotype is gene targeting (Mansour et al., 1993), in which homologous recombination of newly introduced DNA sequence (i.e., the targeting sequence or construct) and a specific targeted DNA sequence residing in the chromosome results in the insertion of a portion of the newly introduced DNA sequence into the targeted chromosomal DNA sequence. This method is capable of generating animals of any desired genotype, and is especially useful for gene disruption (i.e., to “knock out”) at a specific chromosomal gene sequence by inserting a selectable marker into the gene or completely replacing the gene with another nucleotide sequence.

To knock out a genomic sequence, a cloned fragment must be available and intron-exon boundaries within the fragment defined (Mansour et al., 1993). Typically, the targeting construct contains a selectable marker such as Neo (neomycin resistance, see Mansour et al., 1993) flanked by sequences homologous to the chromosomal target DNA, and beyond one of these flanking sequences the herpes simplex virus thymidine kinase gene (HSV-TK, see generally, McKnight et al., 1980). The targeting construct is introduced, e.g., by electroporation, into embryo-derived stem (ES) cells where homologous recombination results in an insertion of the Neomycin resistance marker (Neo), but not the HSV-TK gene, into the targeted chromosomal DNA sequence. The altered ES cells are neomycin resistant and HSV-TK- and so are able to grow in the presence of both G418 and gancyclovir antibiotics. Random insertions contain the HSV-TK gene and are thus sensitive to gancyclovir (Mansour, et al.). Positive ES clones are then microinjected into blastocysts to generate germ-line chimeric mice, which are then bred to obtain progeny that are homozygous for the knock out gene. Such general methods of generating knock out animals have been demonstrated using mice. Genes in other animals such as rats, guinea pigs, gerbils, hamsters, and rabbits, can also be used as long as sufficient DNA sequence data are available to make an appropriate targeting construct to knock out the gene of interest.

Although ato and Math1 share a high degree of sequence conservation, there was an apparent discrepancy between their expression patterns and the consequences of their loss of function. Whereas ato is expressed primarily in the PNS of the fly and its absence causes loss of almost all CHOs (Jarman et al., 1993), Math1 is expressed in the CNS and its loss leads to absence of cerebellar granule neurons, the largest neuronal population in the CNS (Ben-Arie et al., 1997). To better understand the functional relations between ato and Math1, the present invention describes generation of a second Math1 null allele in mice (Math1^{β-gal/β-gal}) by replacement of the Math1 coding region with a β-galactosidase gene (lacZ) and performing a subsequent search for CNS expression of ato in the fruit fly. The Examples describe a functional link between ato and Math1: ato is expressed in the fly brain, and lacZ expression under the control of Math1 regulatory elements (Math1/lacZ) not only replicated the known expression pattern in the CNS (i.e., the neural tube, spinal cord and cerebellum), but appeared in many other cells of the murine PNS. Overexpression of Math1 in Drosophila caused ectopic CHO formation, providing further evidence that ato and Math1 are functionally conserved.

The connections and consistency of the relationship between atonal in Drosophila and Math1 in the mouse suggests that their use as model systems in the art is justified. A family of homologues have been cloned and analyzed in the mouse including MATH1, 2, 3, 4A, 4B, 4C and 5 (Azakawa et al., 1995; Bartholoma and Nave, 1994; Ben-Arie et al., 1997; Ben-Arie et al., 1996, Fode et al., 1998; Ma et al., 1998; McCormick et al., 1996; Shimizu et al., 1995; Takebayashi et al., 1997). It has been suggested that Math1 and Math5 are the only true ato homologues given their amino acid sequence criteria, sharing 67% and 71% identity with the bHLH domain of ATO, respectively (Ben-Arie et al., 2000). A Xenopus atonal homolog, Xath1 has been ectopically expressed in Drosophila and shown to behave similarly to ato (Kim et al., 1997). Furthermore, the ability of Math1 to induce ectopic CHO formation and to restore CHOs to ato mutant embryos (see Example 13) is strong evidence that Math1, and particularly its basic domain, encodes lineage identity information not unlike that encoded by ato and that mammalian cells expressing Math1 are functionally similar and perhaps evolutionarily related to Drosophila cells that require ato. Thus, the similarities between atonal in Drosophila, Xath1 in Xenopus and Math1 in the mouse indicate that these animals are comparable animal model systems. Furthermore, the widespread use of mice in particular as a model system for humans also suggests that it similarly would allow utilization of the invention in humans.

With advances in molecular genetics now standard in the art, sequences from humans and other species can be used interchangeably in a variety of organisms. For example, the rat inducible hsp70 gene was used to produce transgenic mice that overexpressed inducible hsp70, allowing organs from transgenic mice to be protected from ischemic injury (Marber et al. J. Clin. Invest. 95:1446-1456 (1995)) due to the increase in rat hsp70. Sequences in other animals have been interchanged including between humans and rodents to develop rodent models to study human disease, i.e. neurodegenerative diseases. One such example is the expression of the human SCAL gene, which encodes ataxin-1, in mice (Burright, E. N. et al. Cell 82:937-948 (1995)). Transgenic mice were generated expressing the human SCA1 gene with either a normal or an expanded CAG tract. The data illustrated that the expanded CAG repeats were expressed in sufficient amounts in the Purkinje cells to produce degeneration and ataxia. This example illustrates that a mouse model can be established to study spinocerebellar ataxia type 1, which is an autosomal dominant inherited neurologic disorder. In addition to developing mouse models, Drosophila is a hallmark model system in the field. Warrick et al. (1999) produced transgenic flies which co-expressed human hsp70 and a human mutant polyglutamine (MJDtr-Q78). Expression of the human mutant polyglutamine MJDtr-Q78 alone in the flies resulted in the formation of large aggregates in neurons. However, co-expression with human hsp 70 resulted in suppressed aggregation. These examples illustrate that interchangeability of genes is routine in the field of molecular genetics and model systems provide powerful tools to characterize gene function.

### EXAMPLE 3

### Generation of Transgenic Math1 Mice

To detect subtle Math1 expression patterns not identified by RNA in situ hybridization, and thus further illuminate this gene's role during embryonic development, Math1 null alleles (Math1^{β-Gal/β-Gal}) were generated by replacing the Math1 coding region with β-galactosidase (^{β-Gal}).

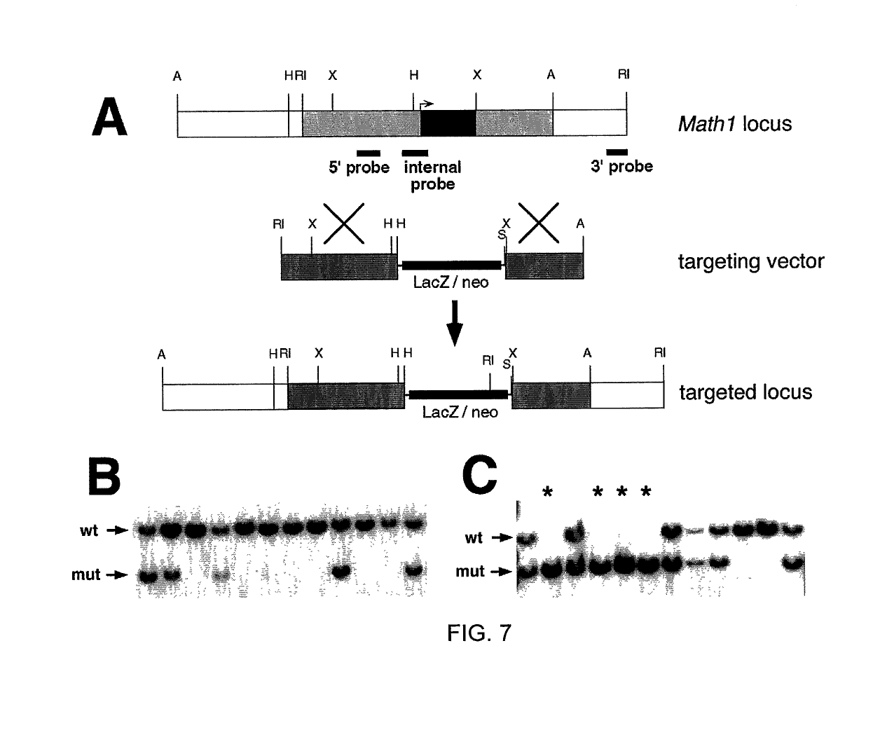

The targeting construct, containing a lacZ cassette and a PGK-neo cassette (FIG. 7A), was used to replace the Math1 coding region. To delete the entire coding region of Math1, a targeting construct was generated that contained the 5′ and 3′ genomic flanking fragments as described previously (Ben-Arie et al., 1997) flanking a pSAβgal/PGK-neo cassette (Friedrich and Soriano, 1991). The construct is designed so that lacZ expression is driven by endogenous Math1 control elements, while an independent PGK promoter drives the expression of the selectable marker neo.

The construct was electroporated into ES cells and selection for neo was achieved with G418. Fourteen out of 76 (18%) clones underwent homologous recombination. Genotyping of ES cells, yolk sac and tail DNA was performed using Southern analysis of EcoR I digested DNA and probes previously described (Ben-Arie et al., 1997). The targeting construct was electroporated into embryonic stem (ES) cells; 14/76 (18%) clones exhibited correct homologous recombination at the Math1 locus (FIG. 7B).

Three ES cell lines carrying the Math1^{+/β-gal} allele were injected into host blastocysts to generate chimeric mice. Math1^{+/β-gal} mice were identified and intercrossed to generate homozygotes (FIG. 7C). The Math1 deletion was confirmed by Southern analysis using both flanking and internal probes (FIG. 7A).

Math1^{β-Gal/β-Gal} mice show all the phenotypic features reported in the Math1^{-/-} mice (Ben-Arie et al., 1997; 2000).

### EXAMPLE 4

### X-Gal Staining, Histological and Immunohistochemical Analyses

Embryos were staged by vaginal plug, with the morning of the plug designated E0.5. Embryos were dissected out of the uterus, separated from extraembryonic membranes, and placed in cold phosphate buffered saline (PBS). The embryos were then fixed in 4% paraformaldehyde (PFA) in PBS for 30 minutes, and washed in cold PBS. Yolk sacs or tails were collected before fixation for DNA extraction and genotyping. Equilibration to improve the penetrability of the staining reagents was performed in 0.02% NP40, 0.01% sodium deoxycholate in PBS for 10 minutes at room temperature. Whole mount staining with X-gal (Bonnerot and Nicolas, 1993) was performed for 16-24 hours at 30° C. while shaking in the same equilibration buffer, which also contained 5 mM potassium ferricyanide, 5 mM potassium ferrocyanide, and 40 mg/ml X-gal (dissolved in DMSO). When the desired intensity of staining was achieved, usually within 18 hours, embryos were washed in PBS, postfixed for 30 minutes in buffered formalin, serially dehydrated in 25, 50, and 70% ethanol, and stored at 4° C.

For histological analysis embryos were further dehydrated in 80, 90, and 100% ethanol, treated in Histoclear (National Diagnostics), and embedded in Paraplast (Oxford Labware). Seven to 20 μm sections were cut using in a microtome (Microme). Counterstaining was performed using nuclear fast red (Vector Laboratories). Immunohistochemistry was performed as detailed previously (Ben-Arie et al., 1997). Antibodies: Anti-cytokeratin 18 (DAKO) 1:20; Anti-human Chromogranin A (DAKO) 1:100; Anti-MATH1 (see below) 1:200.

### EXAMPLE 5

### Expression Patterns in Transgenic Math1 Mice

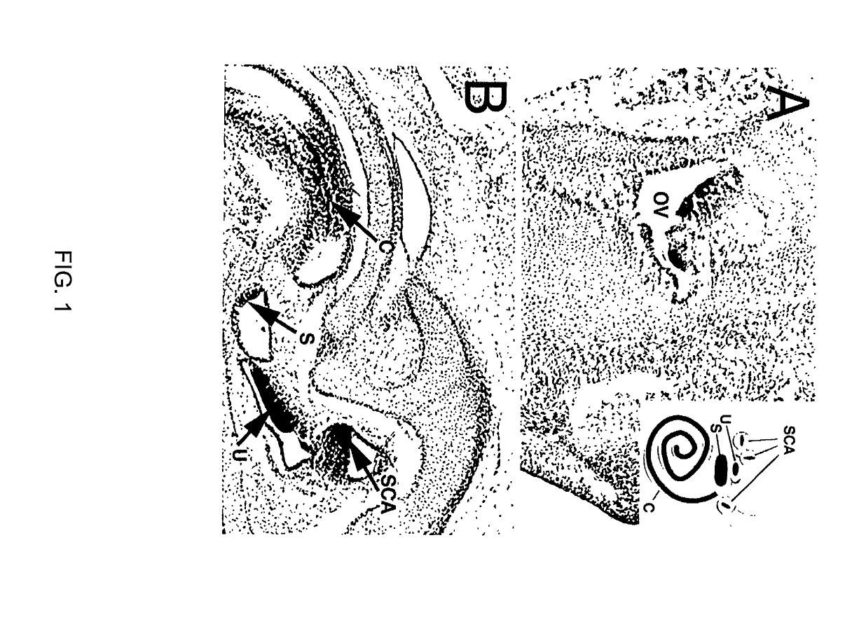

As expected, β-Gal expression in the cerebellum and dorsal spinal cord is identical to that of Math1, and interestingly, β-Gal is also expressed throughout the otic vesicle epithelia at E12.5 and in the sensory epithelia of the utricle, saccule, semicircular canals, and cochlea at E14.5 and E15.5 (FIGS. 1A and 1B). Utricles were obtained from C57BL/129SVEV mice.

Gross morphological analysis of the inner ear of Math1^{β-Gal/β-Gal} mice at E18.5, one day before full gestation, revealed no obvious defects in overall structure and size compared with wild type (wt) littermates. The branches of the VIIIth cranial nerve were present and reached the epithelia, but degenerated due to absence of the hair cells.

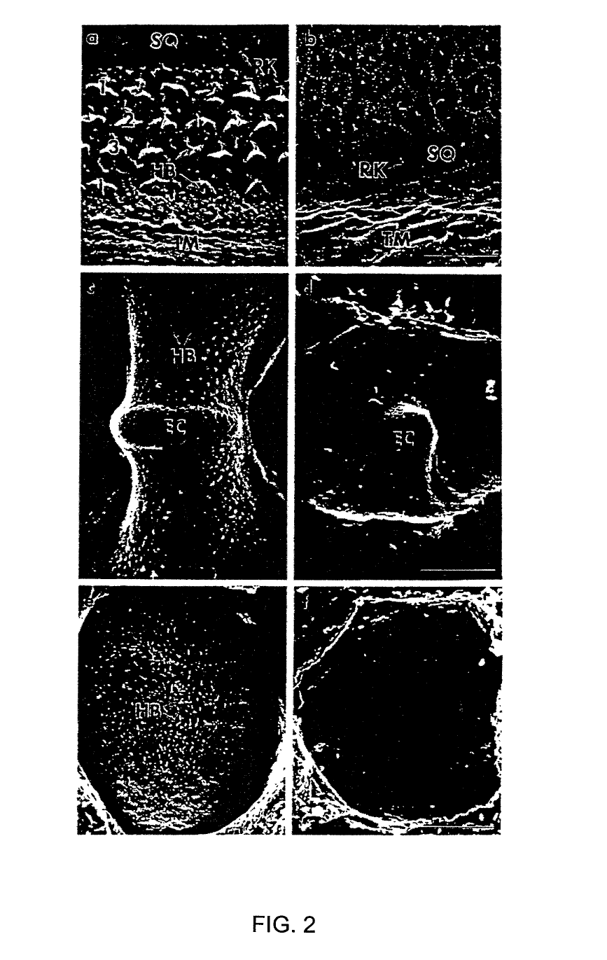

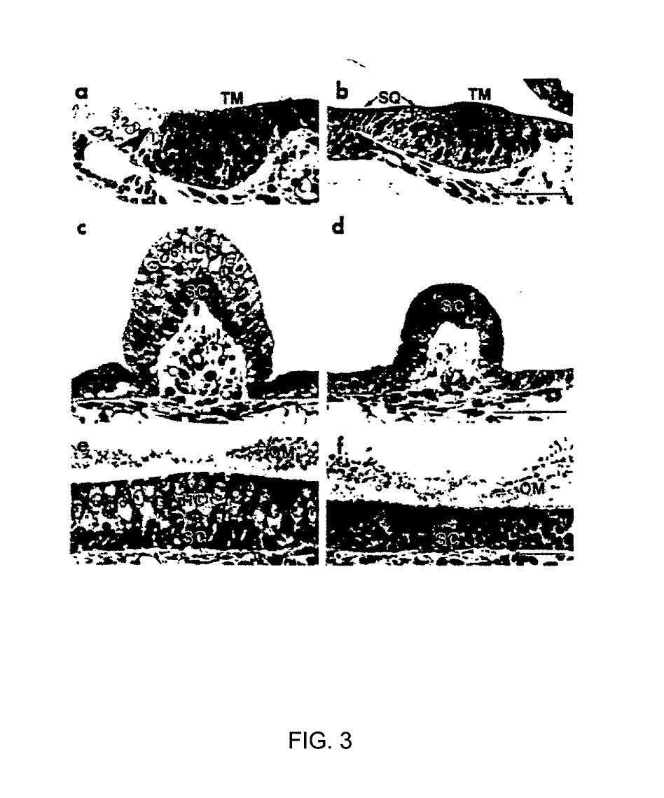

The sensory epithelia were examined in detail. The utricles and cochleas of wild-type, Math1^{+/β-Gal}, and Math1^{β-Gal/β-Gal} mice were excised to allow viewing of the sensory epithelia with Nomarski optics. Hair bundles were present in both organs of wild-type and heterozygotes, but were completely absent in Math1 null litter-mates. Scanning electron microscopy (SEM) of the cochlea and vestibular organs confirmed the absence of hair bundles in null mice (FIGS. 2A through 2F). To determine whether lack of hair bundles reflects the absence of hair cells, cross-sections of the sensory epithelia of all inner ear organs using both light and transmission electron microscopy (LM and TEM, respectively) were examined (FIGS. 3A through 3F). LM and TEM were carried out as described previously (Lysakowski and Goldberg, 1997). Tissue preparation for SEM consisted of osmication (1% OsO₄ in cacodylate buffer), dehydration, critical-point drying, sputter-coating with gold, and examination in a JEOL 35S electron microscope.

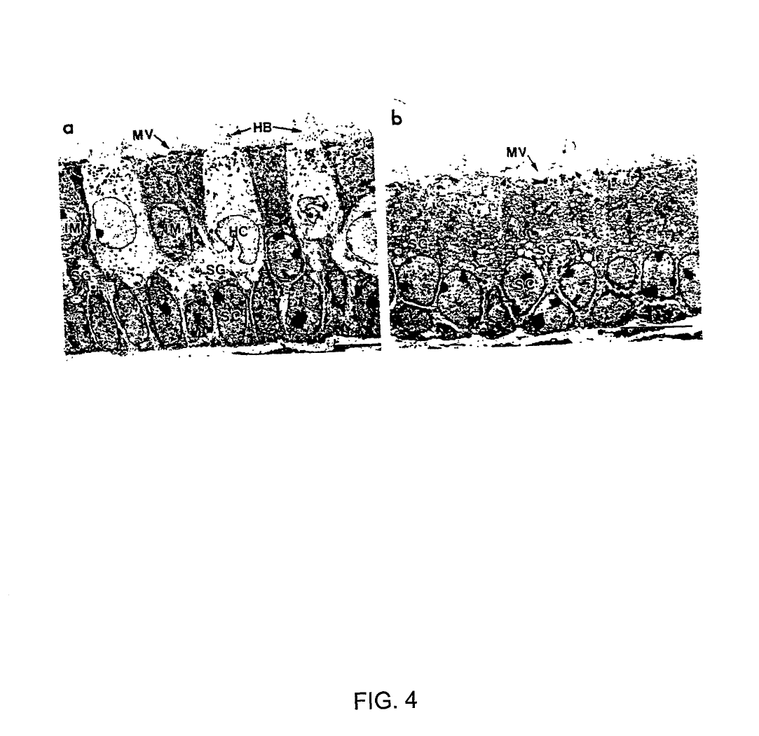

Light microscopy revealed that sensory epithelia in null mice are considerably thinner, lack the normal stratification of cell nuclei and stain uniformly, all of which are consistent with the absence of hair cells. TEM clearly distinguishes between hair cells and supporting cells in normal utricles: hair cells have hair bundles, less electron-dense cytoplasm, more apical nuclei, and no secretory granules (FIGS. 4A and 4B). The sensory epithelia of the null mutants lack hair cells entirely but do have supporting cells with normal appearance (Rüsch et al., 1998), including electron-dense cytoplasm, basal nuclei, and secretory granules. However heterozygous Math1^{+/β-Gal} mice retain hair cells.

### EXAMPLE 6

### Expression of a Hair Cell Specific Marker in Transgenic Math1 Mice

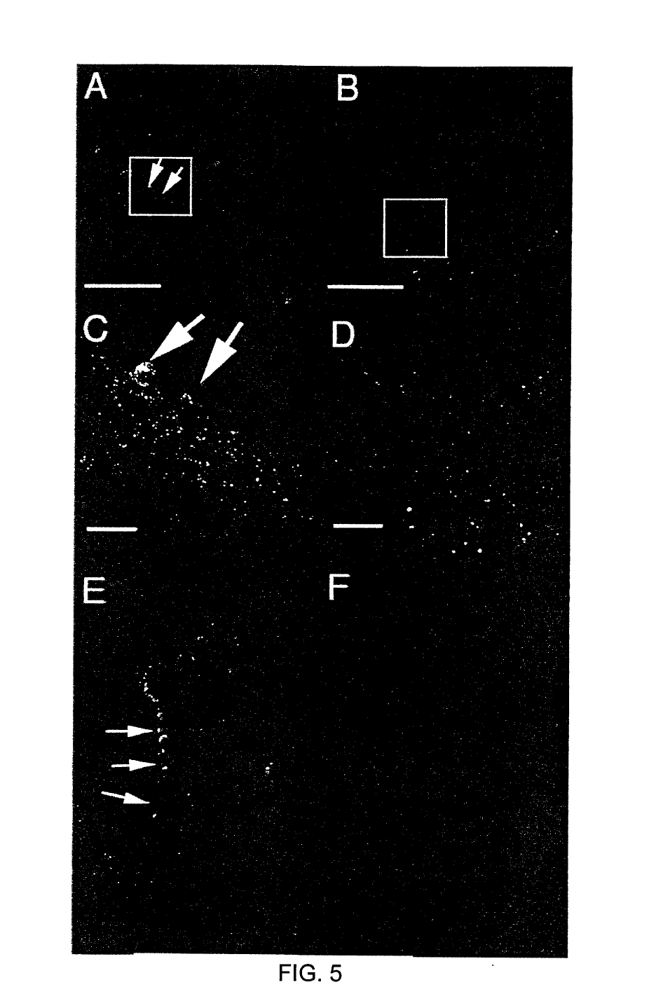

Lack of hair cells at E18.5 can be due to (1) lack of sensory cell progenitors, (2) the inability of progenitors to differentiate into hair cells, or (3) the inability of hair cells to maintain the differential states, as has been observed in the absence of the POU domain transcription factor Brn3c. The first possibility is unlikely because progenitors give rise to both hair cells and supporting cells. To evaluate the remaining possibilities, the expression of the hair cell specific marker, calretinin and myosin VI were examined. Calretinin is a member of the calcium binding family of proteins and is expressed in differentiating hair cells (prior to hair bundle formation) and mature inner ear and auditory hair cells, but not in supporting cells. Calretinin expression in Math1^{β-Gal/β-Gal} and wild-type mice was studied by immunofluorescense on coronal sections of E15.5, E16.5 and E18.5 embryos (FIGS. 5A through 5F).

For immunofluorescence, embryos were fixed for 1.5 hours in 4% paraformaldehyde/PBS at 4° C., sunk through 15% sucrose/PBS for 5 hours then 30% sucrose/PBS overnight, and snap frozen in a 2-methylbutane dry ice bath. 14 μm sections were cut on a cryostat and mounted onto gelatin-coated slides. Sections were fixed onto slides by dipping for 10 minutes in Streck tissue fixative (Streck laboratories) and air drying. Sections were blocked in 30% normal goat serum and 0.3% triton X-100 in PBS for 1 hr at room temperature (RT). Rabbit anti-calretinin polyclonal antibody (Chemicon laboratories) was diluted 1:200 in blocking solution and incubated overnight on sections at 4° C. Sections were washed 3 times (20 minutes each) in Phosphate-Buffered Saline (PBS) at RT. The secondary antibody anti-rabbit antibody, Alexa 488 (Molecular Probes), was diluted 1:400 in blocking solution and used to detect calretinin. Sections were covered and incubated at RT for 2 hours before washing and mounting in Vectashield containing DAPI (Vector). For confocal microscopy, sections were treated with 25 μg/ml RNAse before counterstaining with 50 μg/ml of propidium iodide and mounted in Vectashield without DAPI. Stained sections were viewed under a Bio-Rad 1024 confocal microscope.

Calretinin-positive cells are clearly visible in the sensory epithelia of the semicircular canals and utricles of wild-type mice, but Math1^{β-Gal/β-Gal} embryos lack calretinin expression at all three states. Using the mouse model disclosed herein the present inventors demonstrate that hair cells never develop within the sensory epithelia of Math1^{β-Gal/β-Gal} mice. The presence of the tectorial and otolithic membranes (secreted in part by the supporting cells), together with the TEM results, suggests that the remaining cells in the sensory epithelia of the Math1^{β-Gal/β-Gal} mice are functional supporting cells.

### EXAMPLE 7

### Math1/LacZ Expression Mimics Math1 Expression in the Developing CNS

The developing cerebellum at E14.5 and postnatal day 0 (P0) in Math1^{+/β-gal} and Math1^{β-gal/β-gal} mice were analyzed by RNA in situ hybridization analysis.

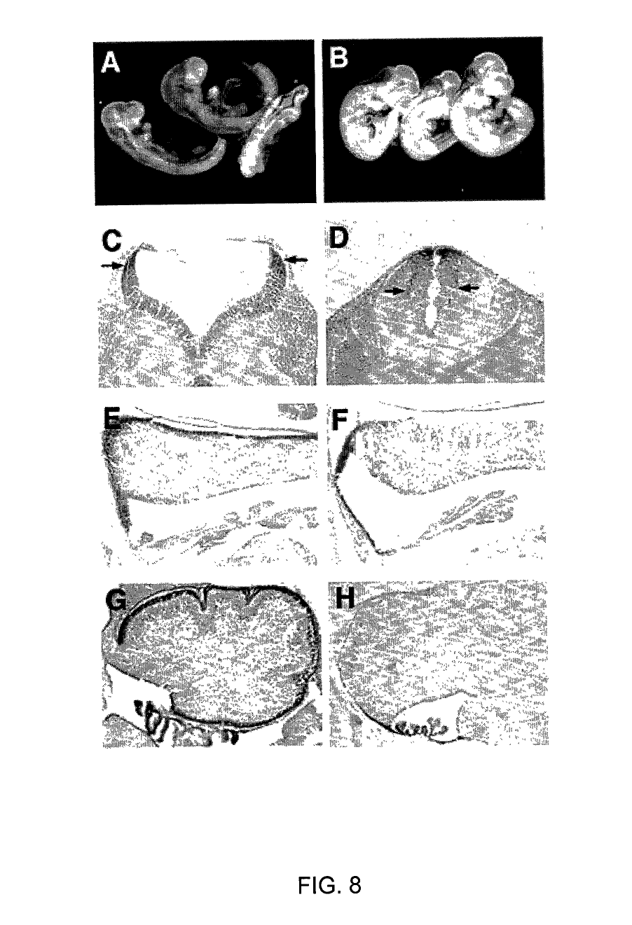

The analysis showed that the expression pattern of the lacZ gene faithfully reproduced the Math1 expression pattern observed by RNA in situ hybridization analysis shown previously (Akazawa et al., 1995; Ben-Arie et al., 1996) (FIGS. 2A, B, E, G). Moreover, the cerebellar phenotype in Math1^{β-gal/β-gal} mice (FIGS. 8F and 8H) was identical to that observed in Math1 null mice (Ben-Arie et al., 1997). At E14.5, the precursors of the EGL are present in the rhombic lip from which they migrate over the cerebellar anlage to populate the EGL (FIG. 8E). Mutant mice displayed far fewer of these cells than heterozygous mice (FIG. 8F). At P0, the neurons of the external granule layer (EGL) were completely lacking (FIG. 8H).

Math1/lacZ expression in the developing hind brain and spinal cord similarly reproduced the expression pattern of Math1 (FIGS. 8C, 8D). The only notable difference between the expression patterns established by in situ hybridization and lacZ staining is that b-galactosidase expression persists in differentiating or migrating cells of the spinal cord because of the stability of the β-GAL protein (FIG. 8D). In summary, the neural tissue expression pattern and cerebellar phenotype associated with the replacement of the Math1 coding region by lacZ is consistent with previously published data on Math1 expression (Akazawa et al., 1995; Ben-Arie et al., 1997; Ben-Arie et al., 1996; Helms and Johnson, 1998), demonstrating that the endogenous control elements were not disrupted by insertion of the lacZ gene. Moreover, many previously undetected clusters of lacZ-expressing cells became apparent upon X-gal staining of whole embryos and sections in Math1^{+/β-gal} mice (see below). It is likely that limitations in the spatial resolution of RNA in situ hybridization techniques used to detect the transcript in earlier studies prevented these sites of expression from being discerned (Akazawa et al., 1995; Ben-Arie et al., 1996). Alternatively, the stability of the lacZ gene product and the increased sensitivity due to signal amplification allowed us to identify sites of relatively low expression levels.

### EXAMPLE 8

### Math1/LacZ is Expressed in Inner Ear Sensory Epithelia

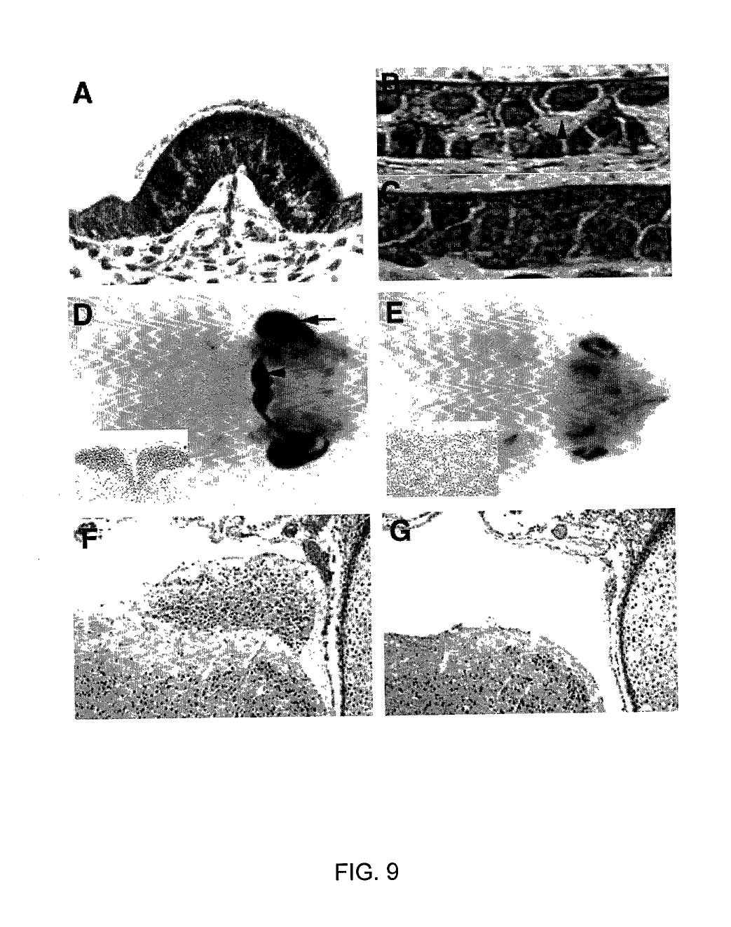

The sensory organs of the inner ear were among the newly identified sites of Math1/lacZ expression, demonstrated utilizing the methods described in Example 2. Expression in the otic vesicle was first detected at E12.5 and continued until E18.5 throughout much of the sensory epithelia (Bermingham et al., 1999) (FIGS. 9A, 9B). Null mutants displayed Math1/lacZ expression in the inner ear throughout embryogenesis (FIG. 9C). Math1 null mutants lack hair cells in all of the sensory organs (Bermingham et al., 1999), but maintain supporting cells, the other sensory epithelia-derived cells (FIG. 9C). These supporting cells seem to be functional, based on their morphology and the presence of overlying membranes secreted in part by these cells. Although the expression of Math1 in inner ear sensory epithelia was not demonstrated by RNA in situ hybridization analysis, the complete lack of inner ear hair cells in the null mutants leaves little doubt about the authenticity of the Math1/lacZ expression pattern.

Math1 is clearly essential for hair cell development in the inner ear. Its expression pattern and in vivo function are akin to those of Math1's proneural homolog, atonal (ato) (A. P. Jarman, Y. Grau, L. Y. Jan, Y. N. Jan, Cell 73, 1307-21 (1994)). ato is expressed in a ring of epithelial cells within the antennal disc of Drosophila. Some of these epithelial cells will subsequently develop into mechanoreceptors in the Johnston organ, which is necessary for hearing and negative geotaxis. It is interesting to note that mechanoreceptor progenitor cells are absent in ato mutants, whereas only the mechanoreceptors, and not their progenitors, are absent in Math1 null mice.

Based on the observations made herein, the present inventors have recognized that Math1 is required for the specification of inner ear hair cells. In a sense, Math1 acts as a “pro-hair cell gene” in the developing sensory epithelia. In conjunction with two recent studies, the present inventors have recognized that the results provided herein provide evidence supporting a lateral inhibition model for the determination of hair cells and supporting cells (Haddon et al., 1998; Adam, et al., 1998), in which the interplay of Delta, Notch, and Serratel results in the selection of individual hair cells from clusters of competent cells. Such a model entails that the sensory epithelia express a “pro-hair cell gene” whose function is essential for hair cell fate specification.

The ectopic expression of ato in the fruitfly and its homolog Xath1 in Xenopus (Kim et al., 1997) can recruit epithelial cells into specific neuronal fates, and the expression of Math1 in inner ear epithelia strongly suggests loss of a functional Math1 gene is likely to be a common cause of deafness and vestibular dysfunction.

### EXAMPLE 9

### Math1/LacZ is Expressed in Brain Stem Nuclei

In the brainstem Math1/lacZ staining appeared from E18.5 to P7 in the ventral pons in the regions corresponding to the pontine nuclei (FIG. 9D and inset). This finding is consistent with the hypothesis of Akazawa and colleagues that Math1-positive cells in the developing hind brain are precursors to the bulbopontine neurons (Akazawa et al., 1995). No such staining appeared in the null mutants (FIG. 9E and inset). These data raise the possibility that the absence of lacZ staining in pontine nuclei can be due to failure of their precursors to migrate, proliferate, and/or differentiate. Ventral pontine nuclei were examined upon haematoxylin and eosin staining of sections and were found to be missing in the brain stem of null mice (FIGS. 9F, G). Furthermore, the failure of null mouse newborns to breathe can be due to absence of these brainstem neurons.

### EXAMPLE 10

### Math1/LacZ is Expressed in Chondrocytes

Math1^{+/β-Gal} heterozygotes displayed expression of Math1 in articular cartilage (FIGS. 6A and 6B). FIG. 6A demonstrates expression in all joints of a forelimb. Upon closer examination of an elbow joint, Math1 is noted to be expressed exclusively in the non-ossified articular chondrocytes.

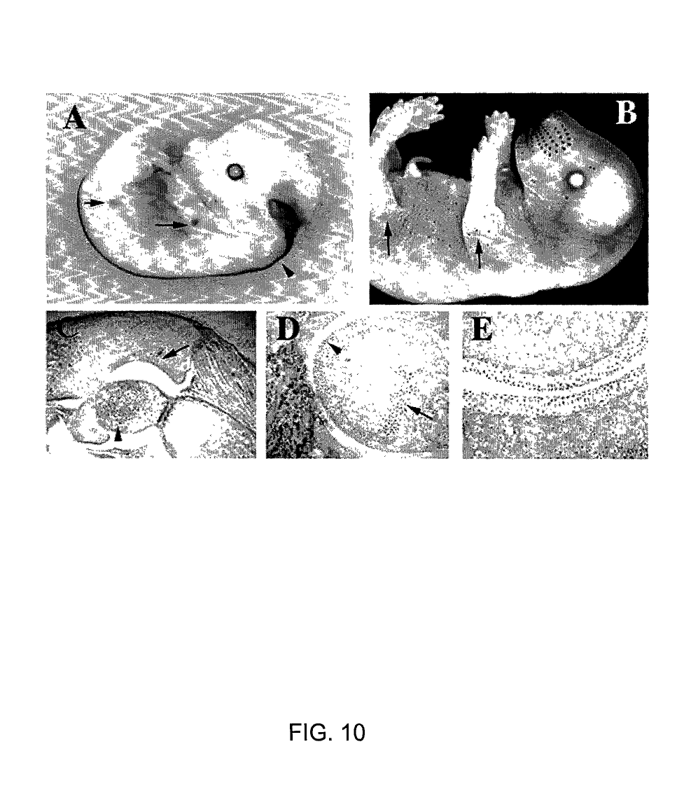

Expression of Math1/lacZ was detected in the developing proximal joints, such as those of the hip and shoulder, as early as E12.5 (FIG. 10A). X-gal positive staining was detected at subsequent developmental stages in a progressive proximal-distal pattern that paralleled the normal development of joints (FIG. 10B). In the joints, Math1/lacZ expression immediately follows mesenchymal condensation, which begins at E11.5. Condensed mesenchyme cells differentiate into chondrocytes (Bi et al., 1999; Horton et al., 1993; Karsenty, 1998).

Chondrocytes differentiate in three major phases during bone formation: resting, proliferating and hypertrophic. The resting chondrocytes that populate the articular cartilage are referred to as articular chondrocytes (Buckwalter and Mankin, 1998; Poole, 1997). Prior to birth, resting chondrocytes constitute the entire chondrocyte population in joints. To establish which cells expressed Math1/lacZ, sections from E18.5 and P7 Math1^{+/β-gal} mice were stained with X-gal. Math1/lacZ is expressed in the resting chondrocytes of all joints analyzed at E18.5; resting chondrocytes in the elbow joint are shown in FIG. 10C, and FIG. 10D shows the resting, proliferating, and articular chondrocytes of a P7 mouse.

The joints of E18.5 embryos were examined with anti-MATH1 antibody prepared by the following methods. An EcoR I-Hind III fragment encoding the N-terminal 156 amino acids of the Math1 open reading frame (Math1D) was cloned into the pET 28a+expression vector (Novagen). Math1D fragment was expressed as a His tag fusion protein. Soluble MATH1D protein was purified according to His-tag kit specifications (Novagen) and 2 mg of protein were used to immunize Chickens (Cocalico Biologicals Inc.).

Expression was found in resting chondrocytes, whereas no expression was observed in null embryos. It should be noted that not all articular cartilage cells express Math1/lacZ (FIG. 10E). Math1/lacZ expression in Math1 null mutants is similar to that in heterozygous mice at E18.5, suggesting that Math1 is not required for resting chondrocyte development.

### EXAMPLE 11

### Math1/LacZ is Expressed in Merkel Cells

By E14.5 Math1/lacZ-positive cells were apparent around the vibrissae and in the skin of much of the body (FIG. 10B). In the trunk, the stained cells were arranged in a striped pattern defined by the epidermal ridges. This staining was apparent only in the hairy, not the glabrous, skin. All the primary (mystical) vibrissae, including the lateral nasal, maxillary and four large hairs, were positive for Math1/lacZ. Staining was also detected in the secondary vibrissae, including the labial, submental, rhinal, and isolated orbital vibrissae (supra-, infra- and post-orbital) (Yamakado and Yohro, 1979). By E15.5 staining appeared in clusters of cells in the foot pads (FIG. 10B).

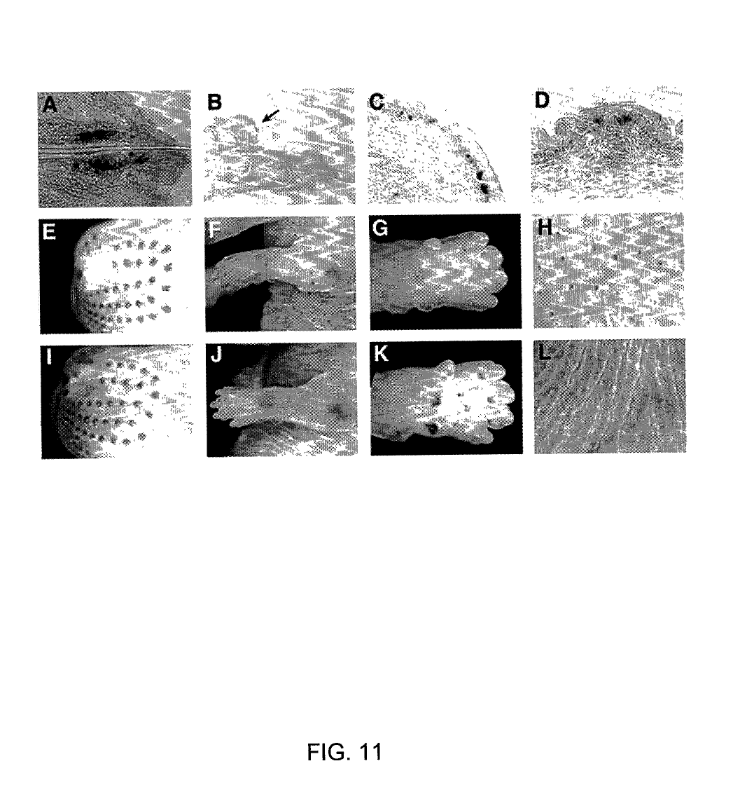

To identify the Math1/lacZ-positive cells in the vibrissae, footpad, and hairy skin, histological sections from Math1^{+/β-gal} mice were examined (FIGS. 11A-D). Sections through the vibrissae showed that the stained cells are localized to the more apical half of the hair shaft, but are not in the hair itself. Cross sections through the foot pad illustrated staining of cluster of cells in the epidermal layer (FIGS. 11B, C). As shown in FIG. 11D, sections through the truncal skin identified clusters of Math1/lacZ-stained cells. The stained cells were arranged in a horseshoe-shaped pattern centered within an elevated button-like structure in the hairy skin. These button-like structures were identified as touch domes or Haarscheiben (Pinkus, 1905), which are characterized by a thickened epidermis and an elevated dermal papilla with a capillary network. Touch domes are associated with large guard hairs dispersed between other hair types in the coat. The spatial distribution of Math1/lacZ-stained cells, the timing of their appearance at E14.5, and their localization within the mystical pads of the vibrissae and the touch domes in the hairy skin suggest that these cells correspond to Merkel cells, specialized cells in the epidermis that form slow-adapting type I mechanoreceptor complexes with neurites (Munger, 1991).

The results of comparative analysis of the Math1/lacZ expression pattern in heterozygous and homozygous E16.5 animals are shown in FIGS. 11E-L. Math1^{β-gal/β-gal} embryos displayed a staining pattern similar to that of Math1^{+/β-gal} littermates in the vibrissae and footpads (FIGS. 11E-G, I-K). In contrast, staining in the touch domes of the hairy skin was barely detectable in Math1^{β-gal/β-gal} embryos (FIGS. 11H-L). The reduction of staining in null animals was also obvious at E18.5.

To further define Math1/lacZ-positive cells in the skin, Math1^{+/β-gal} mice were mated to Tabby mice. Tabby (Ta) is a spontaneous X-linked mutation displaying a similar phenotype in hemizygous males and homozygous females (Ferguson et al., 1997). Tabby mutants lack hair follicles (tylotrich), a subset of Merkel cells that are associated with touch domes in the hairy skin of the trunk (Vielkind et al., 1995), and some of the five secondary vibrissae on the head (Gruneberg, 1971). Hence, in a cross of Ta/Ta females with a heterozygous Math1^{+/β-gal} male, 50% of the male progeny are Ta/Y: Math1^{+/β-gal}, allowing us to assess whether the Math1/lacZ-positive cells correspond to Merkel cells.

Ta/Ta females were time-mated with Math1^{+/β-gal} males, and embryos were harvested at E16.5. Each pup's gender was determined by PCR on tail DNA, using primers (forward 5′-TGAAGCTTTTGGCTTTGAG-3′; SEQ ID NO: 67, and reverse

5′-CCGCTGCCAAATTCTTTGG-3′; SEQ ID NO:68) that yielded a 320 bp product from chromosome X, and a 300 bp product from chromosome Y (Liu et al., 1999). Amplification conditions were: 92° C./1 min, 55° C./1 min, 72° C./1 min for 32 cycles, with an initial denaturation step of 94° C./7 min and last extension step of 72° C./7 min. Amplification products were separated on 2% agarose gels. X-gal-stained embryos were scored independently by 2 individuals, and only then were results matched with the determined gender.

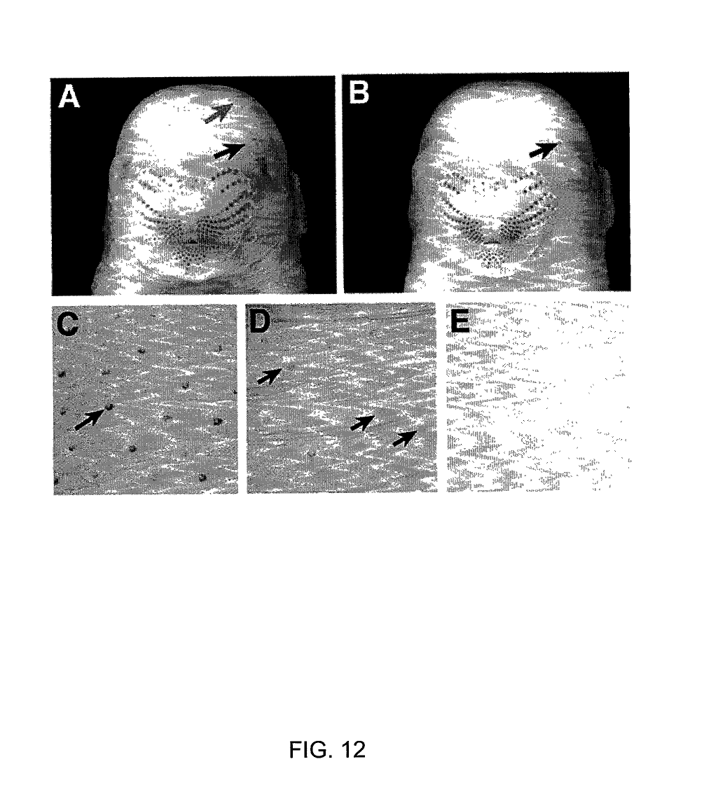

Both Tabby females and males carrying the Math1^{+/β-gal} allele displayed X-gal staining in the vibrissae and foot pads (FIGS. 12A, B). The effect of the Tabby mutation on the number of secondary vibrissae was quite clear: hemizygous males completely lacked Math1/lacZ-positive cells in the secondary vibrissae (typically lacking in Ta mutants) and on the trunk (FIG. 12E). Females that are heterozygous for Tabby showed patchy staining in the touch domes (although less than wt), as should be anticipated in female carriers of a mutation in a gene that undergoes random X chromosome inactivation (FIGS. 12C, 12D). The localization and distribution of the positive cells, as well as their absence in selected vibrissae and the trunk of Tabby males, strongly indicate that Math1 is expressed in the Merkel cells associated with guard follicles in the touch domes of the hairy skin.

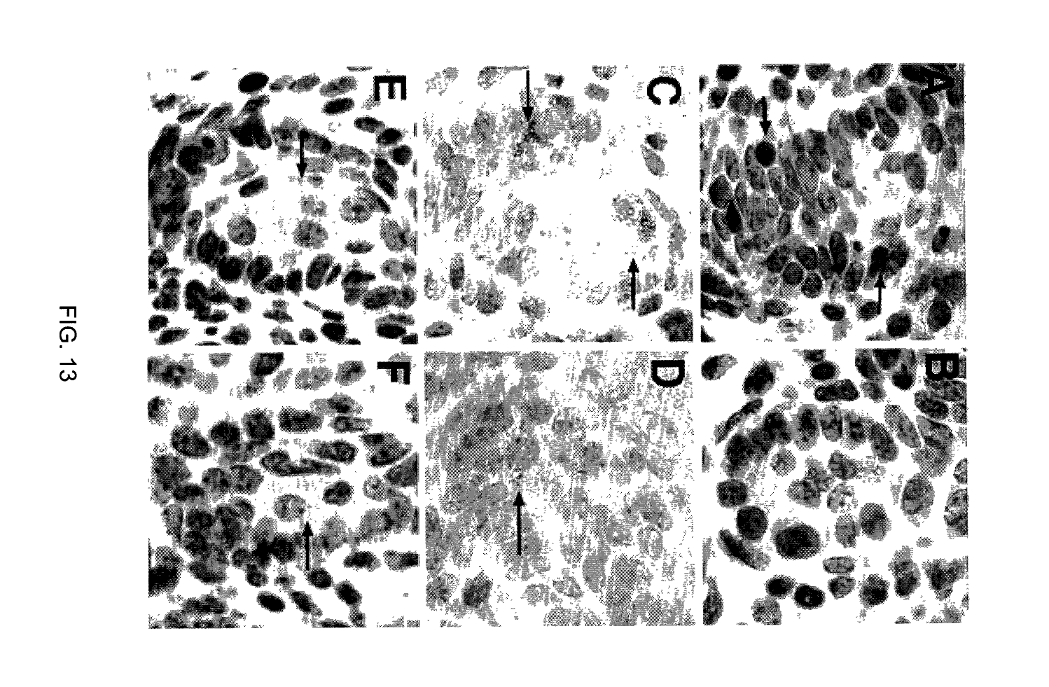

To ascertain whether Math1/lacZ staining pattern reflects normal Math1 expression pattern, immunohistochemical analysis of MATH1 was performed on sections from abdominal skin (see Example 2). As seen in FIGS. 13A and B, MATH1-positive cells were detected around the hair follicles of Math1^{+/+} but not Math1^{β-gal/β-gal} mice. Antibodies against two Merkel cells markers were chosen for further analysis: anti-cytokeratin18, expressed in simple epithelia, and chromogranin, localized to secretory granules of neuroendocrine, endocrine, and neuronal tissues. Both cytokeratin 18 (FIGS. 13C,D) and chromogranin A (FIGS. 13E, 13F) confirmed the identity of the Math1/lacZ-positive cells as Merkel cells, but did not reveal staining abnormalities in Math1^{β-gal/β-gal} mice. Thus, Math1 does not seem to be essential for the genesis of the neuroendocrine Merkel cells, in contrast to pure neuronal cell types like cerebellar EGL and pontine nuclei.

### EXAMPLE 12

### Math1 Partially Rescues Chinese Hamster Ovary Cells (CHO) in Flies Deleted for ATO

This Example demonstrates that atonal-associated genes can induce the development of CNS cells in animals deficient in a native atonal-associated gene or gene product. This Example also demonstrates that atonal-associated genes can therapeutically function in species in which they are not natively expressed.

Given the remarkable similarity in expression patterns of ato and Math1, and their identical basic domains, Math1 was tested to see if it would mimic the effects of ato overexpression by producing ectopic chordotonal organs as described by the following methods. Wild-type, also known as yw flies, were transformed with a UAS-Math1 construct as described (Brand and Perrimon, 1993). To overexpress Math1 in wild type flies, yw; UAS-Math1 flies were mated to HS-Gal4 flies. The progeny were heat shocked as previously described (Jarman et al., 1993). To rescue the loss of chordotonal organs in ato mutant flies, w; UAS-Math1/UAS-Math1; ato1/TM6 flies were crossed to w; HS-Gal4/CyO; ato1/TM6 flies. Embryos were collected for 3 hr., aged for 3 hr., heat shocked for 30 min. at 37° C. and allowed to develop for the next 12-15 hr. Embryos were fixed in 4% formaldehyde in PBS with 50% heptane. Embryos were washed with 100% ethanol, transferred to PBT and stained with mAb 22C10 as previously described (Kania et al., 1995) to detect PNS neurons. Chordotonal neurons were identified by their distinct morphology and position.

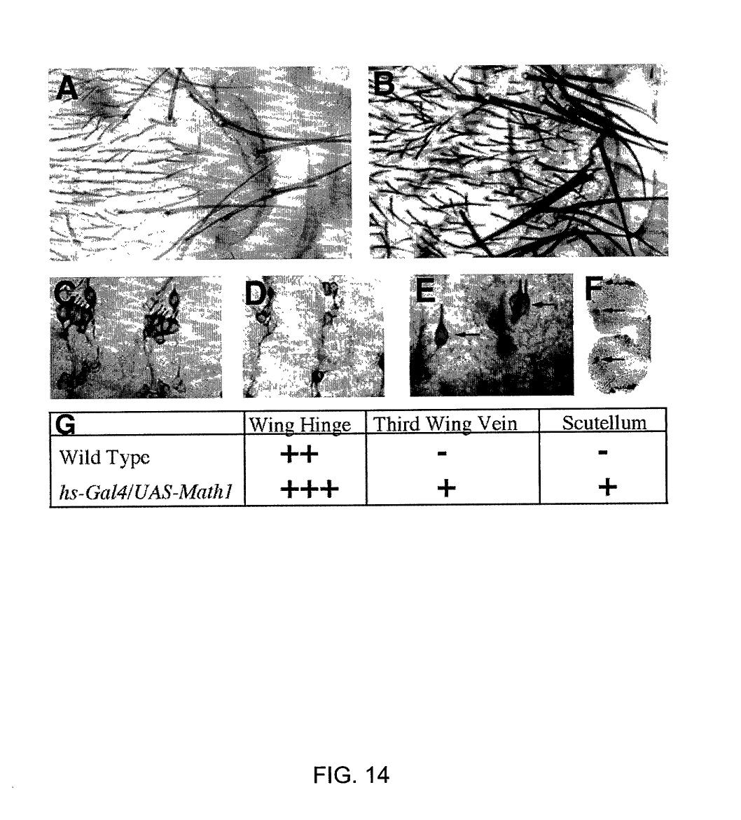

Expressing Math1 during pupal development by heat shock using the UAS-Gal4 system (Brand and Perrimon, 1993) resulted in supernumerary external sense organs on the notum (FIG. 14 A,B) and the wing blade, as reported for ato (Jarman et al., 1993) and the Achaete-Scute complex (AS-C) genes (Brand and Perrimon, 1993; Rodriguez et al., 1990). Math1 expression in flies, like ato, produced ectopic chordotonal organs (FIG. 8G), although with less efficiency. Overexpression of the AS-C genes does not, however, result in ectopic chordotonal organs (Jarman et al., 1993). Math1 thus has a similar functional specificity to ato.

Since several ato enhancers are ato-dependent (Sun et al., 1998), they can be activated by Math1, which would then lead to ectopic CHO specification. To determine whether Math1 can substitute for ato function in the fly, and to rule out the possibility that production of CHOs by Math1 is due to ato activation, Math1 was expressed in ato mutant embryos. The mutants lack all chordotonal neurons (FIG. 14C), but overexpressing Math1 partially rescues the loss of these neurons (FIG. 14D) in a manner similar to ato (Chien et al., 1996).

### EXAMPLE 13

### Significance of Atonal and Math1 in the CNS and PNS

Over the past few years significant progress has been made towards unraveling the roles of bHLH proteins in vertebrate neurogenesis. Neural vertebrate bHLH-encoding genes were isolated and characterized because Drosophila homologues such as ato or the AS-C genes had been previously shown to be required for neurogenesis (Anderson, 1995; Guillemot, 1046 1995; Lee, 1997; Takebayashi et al., 1997). Indeed, several genes were shown to be proneural because their absence caused a failure of neuroblast or sensory organ precursor (SOP) specification, whereas their overexpression lead to the recruitment of supernumerary neuronal precursors (Ghysen and Dambly-Chaudiere, 1989). With the exception of neurogenin (Ngn) 1 and 2 (Fode et al., 1998; Ma et al., 1998), it remains uncertain which of the vertebrate homologues play roles similar to their Drosophila counterparts, and what precise role different bHLH proteins play in neural development. In Drosophila, ato is required for the development of a specific subset of sense organs, the chordotonal organs (Jarman et al., 1993). CHOs are internal mechanosensors of the PNS (McIver, 1985). Thus, ato and the CHOs provide an excellent system in which to ascertain not only the molecular and developmental relationship between invertebrate and vertebrate neurogenesis vis-à-vis the function of the proneural genes, but also the evolutionary conservation of sensory organ function and specification. Seven ato homologues have been cloned and analyzed in the mouse: Mouse Atonal Homologues (MATH) 1, 2, 3, 4A (also known as Ngn2), 4B (Ngn3), 4C (Ngn1), and 5 (Akazawa et al., 1995; Bartholomä and Nave, 1994; Ben-Arie et al., 1997, 1996; Fode et al., 1998; Ma et al., 1998; McCormick et al., 1996; Shimizu et al., 1995; Takebayashi et al., 1997). Most are expressed during neurogenesis in both the CNS and PNS. These homologues vary in the degree of their sequence conservation, and can be divided into three groups. The most distantly related group, the neurogenins, includes Ngn 1, 2 and 3. These gene products share, on average, 53% identity in the bHLH domain with ATO. They are expressed largely in mitotic CNS and sensory ganglia progenitor cells. Recent work suggests that these genes can play a role in neuroblast determination, and can therefore be true proneural genes (Fode et al., 1998; Ma et al., 1998). The second group includes MATH2 and MATH3, which share 57% identity in the bHLH domain with ATO. These proteins have been postulated to function in postmitotic neural cells (Bartholomä and Nave, 1994; Shimizu et al., 1995). Math2 expression is confined to the CNS, while Math3 is expressed in both the CNS and the trigeminal and dorsal root ganglia. The third group includes MATH1 and MATH5, arguably the only true ato homologues by amino acid sequence criteria, sharing 67% and 71% identity with the bHLH domain of ATO, respectively. It is noteworthy that both genes encode a basic domain identical to that of ATO. Interestingly, the basic domain of ATO was shown to be sufficient, in the context of another proneural protein (SCUTE), to substitute for the loss of ato function (Chien et al., 1996). Math1 was initially shown to be expressed in the precursors of the cerebellar EGL and in the dorsal spinal cord (Ben-Arie et al., 1997, 1996). Math5 is expressed in the dividing progenitors in the developing retina and in the vagal ganglion (Brown et al., 1998). With the exception of Math5 expression in the neural retina, these observations pose a paradox: none of the vertebrate homologs appeared to be expressed in peripheral organs or tissues similar to those where ato is expressed. Jarman et al. (1993) reported that ato is expressed in the CNS. In the examples described herein it is shown that, in addition to the inner proliferation center of the optic lobe, ato is expressed in a small anteriomedial patch of cells in each brain lobe (FIG. 8F). Because it remains unclear, however, precisely what role ato plays in Drosophila CNS development, it has been difficult to argue that ato and its vertebrate homologues display functional conservation. The experiments presented herein reveal sites of previously uncharacterized Math1 expression. As expected, Math1/lacZ expression in the CNS corresponds to that of Math1, but Math1 is also expressed in the skin, the joints, and the inner ear, in striking parallel to ato expression in the fly. Moreover, the expression in the ear (sensory epithelium) and the skin (Merkel cells) is restricted to sensory structures whose function is to convert mechanical stimuli into neuronal electrochemical signals. It is important to point out that in Drosophila, ato appears to play two roles simultaneously. It is required not only to select the precursors of the CHOs (proneural role), but also to specify these precursors as CHO precursors (lineage identity role) (Jarman and Ahmed, 1998; Jarman et al., 1993). The specificity of Math1 expression in the periphery makes it tempting to speculate that it, too, can endow specific cells with very specific lineage identities to distinguish them functionally from other sensory structures. The ability of Math1 to induce ectopic CHO formation and to restore CHOs to ato mutant embryos supports the notion that Math1, and particularly its basic domain, encodes lineage identity information not unlike that encoded by ato. This suggests that the mammalian cells expressing Math1, at least in the ear and the skin, are functionally similar and perhaps evolutionarily related to Drosophila cells that require ato. Furthermore, Math5 expression in the neural retina suggests that the functions of atonal in the fly are carried out by two genes in the mouse: the development of some mechanoreceptors is under the control of Math1 and retinal development is possibly under the control of Math5. It is interesting to note that in the fully sequenced nematode C. elegans, only one homolog of atonal, lin-32, was identified (Zhao and Emmons, 1995). Mutants with the u282 allele of lin-32 are touch-insensitive, which strengthens the argument for evolutionary conservation of atonal function in mechanoreception. The pattern of Math1/lacZ expression in the pontine nuclei suggested this region should be carefully evaluated in null mutants. Although no defects in the pons of Math1 null mice (Ben-Arie et al., 1997) were originally detected, closer analysis revealed the lack of pontine nuclei at this site. These neurons derive from the rhombic lip (Altman and Bayer, 1996) as do the EGL neurons, which are also lacking in Math1 null mice. While it is possible to draw parallels between Math1 and ato expression in the skin and ear, it is not clear that such is the case for the joints. ato expression in the fly joints is required for the formation of leg CHOs. In contrast, Math1 is expressed in resting and articular chondrocytes that do not have any described neural function, and for which no parallels exist in the fly. It can be that Math1 expression in cartilage indicates a novel role for a mechanosensory gene, or it can simply reflect similarities in the molecular events underlying the development of the various Math1-expressing cell types. Alternatively, CHOs can also function as joint structural elements in the fly, or articular cartilage can have a mechanoreceptive or transducive capacity yet to be described. There is no evidence at this point to support one or another of these possibilities. Analyzing the functions of ato and Math1 will enhance the understanding of neural development and the evolutionary conservation of sensory function. The sites and specificity of Math1 expression can make it suitable as a tool of gene therapy or gene activation approaches to illnesses such as hearing loss and osteoarthritis that are due to age-related or environmental damage.

### EXAMPLE 14

### Atonal-Associated Nucleic Acid Delivery Using Adenovirus

Human adenoviruses are double-stranded DNA tumor viruses with genome sizes of approximate 36 kb. As a model system for eukaryotic gene expression, adenoviruses have been widely studied and well characterized, which makes them an attractive system for development of adenovirus as a gene transfer system. This group of viruses is easy to grow and manipulate and they exhibit a broad host range in vitro and in vivo. In lytically infected cells, adenoviruses are capable of shutting off host protein synthesis, directing cellular machineries to synthesize large quantities of viral proteins, and producing copious amounts of virus.

The E1 region of the genome includes E1A and E1B, which encode proteins responsible for transcription regulation of the viral genome, as well as a few cellular genes. E2 expression, including E2A and E2B, allows synthesis of viral replicative functions, e.g. DNA-binding protein, DNA polymerase, and a terminal protein that primes replication. E3 gene products prevent cytolysis by cytotoxic T cells and tumor necrosis factor and appear to be important for viral propagation. Functions associated with the E4 proteins include DNA replication, late gene expression, and host cell shutoff. The late gene products include most of the virion capsid proteins, and these are expressed only after most of the processing of a single primary transcript from the major late promoter has occurred. The major late promoter (MLP) exhibits high efficiency during the late phase of the infection.

As only a small portion of the viral genome appears to be required in cis, adenovirus-derived vectors offer excellent potential for the substitution of large DNA fragments when used in connection with cell lines such as 293 cells. Ad5-transformed human embryonic kidney cell lines have been developed to provide the essential viral proteins in trans. The inventors thus reasoned that the characteristics of adenoviruses rendered them good candidates for use in targeting Math1 deficient cells in vivo. In another embodiment these constructs include a HathI or any atonal-associated nucleic acid sequence.

Particular advantages of an adenovirus system for delivering foreign proteins to a cell include: (i) the ability to substitute relatively large pieces of viral DNA by foreign DNA; (ii) the structural stability of recombinant adenoviruses; (iii) the safety of adenoviral administration to humans; (iv) lack of any known association of adenoviral infection with cancer or malignancies; (v) the ability to obtain high titers of the recombinant virus; and (vi) the high infectivity of Adenovirus.

One advantage of adenovirus vectors over retroviruses is a higher level of gene expression. Additionally, adenovirus replication is independent of host gene replication, unlike retroviral sequences. Because adenovirus transforming genes in the E1region can be readily deleted and still provide efficient expression vectors, oncogenic risk from adenovirus vectors is thought to be negligible.

In general, adenovirus gene transfer systems are based upon recombinant, engineered adenovirus that is rendered replication-incompetent by deletion of a portion of its genome, such as E1, and yet still retains its competency for infection. Relatively large foreign proteins can be expressed when additional deletions are made in the adenovirus genome. For example, adenoviruses deleted in both E1 and E3 regions are capable of carrying up to 10 Kb of foreign DNA and can be grown to high titers in 293. Surprisingly, persistent expression of transgenes following adenoviral infection is possible. Use of the adenovirus gene transfer system can be more useful for the delivery of Math1 to cells in nascent or damaged cartilage in joints. In particular, the Math1 adenovirus can be used to deliver Math1, and confer Math1 gene expression in, non-ossified joint cartilage that has been damaged as a consequence of osteoarthritis.

### EXAMPLE 15

### Math1-Adenovirus Constructs

Recombinant virions for the controlled expression of Math1 can be constructed to exploit the advantages of adenoviral vectors, such as high titer, broad target range, efficient transduction, and non-integration in target cells for the transformation of cells into hair cells. In a specific embodiment these constructs include a Hath1 or any atonal-associated nucleic acid sequence. In one embodiment of the invention, a replication-defective, helper-independent adenovirus is created that expresses wild type Math1 sequences under the control of the human cytomegalovirus promoter or the metallothionine promoter.

Control functions on expression vectors are often provided from viruses when expression is desired in mammalian cells. For example, commonly used promoters are derived from polyoma, adenovirus 2 and simian virus 40 (SV40). The early and late promoters of SV40 virus are particularly useful because both are obtained easily from the virus as a fragment which also contains the SV40 viral origin of replication. Smaller or larger SV40 fragments can also be used provided there is included the approximately 250 bp sequence extending from the HindIII site toward the Bg1I site located in the viral origin of replication. Further, it is also possible, and often desirable, to use promoter or control sequences normally associated with the Math1 gene sequence, namely the Math1 promoter, provided such control sequences are compatible with the host cell systems or the target cell. One such target cell is located in the inner ear of a human patient in need of inner ear hair cells.

An origin of replication can be provided by construction of the vector to include an exogenous origin, such as can be derived from SV40 or other viral (e.g., polyoma, adeno, VSV, BPV) source, or can be provided by the host cell chromosomal replication mechanism. If the vector is integrated into the host cell chromosome, the latter is often sufficient.

### EXAMPLE 16

### Atonal-Associated Nucleic Acid Delivery Using Retrovirus

Another approach for gene delivery capitalizes on the natural ability of viruses to enter cells, bringing their own genetic material with them. Retroviruses have promise as gene delivery vectors due to their ability to integrate their genes into the host genome, transferring a large amount of foreign genetic material, infecting a broad spectrum of species and cell types and because they are easily packaged in special cell-lines. Retroviruses can be particularly useful for the delivery of Math1 into inner ear hair cells that have reduced expression of Math1, or that are in need of over-expression of Math1. In another embodiment these constructs include a Hath1 or any atonal-associated nucleic acid sequence.

### EXAMPLE 17

### Math1 Retroviral Constructs

The Math1 open reading frame (ORF) was excised from pBluescript by an EcoR I-XbaI digest. The fragment was gel purified, and blunt ended using Klenow DNA polymerase. The retroviral vector pLNCX (purchased from CLONTECH) was linearized with HpaI, and ligated with the Math1 ORF fragment. The ligation was transformed into transformation competent E. coli cells. The resulting antibiotic resistant colonies were assayed for the presence of the correct construct.

The cloning, reproduction and propagation retroviral expression vectors are well known to those of skill in the art. One example of a retroviral gene transfer and expression system that has been used to express Math1 is the CLONTECH pLNCX, pLXSN and LAPSN expression vectors. For propagation of these vectors PT67 and EcoPack packaging cell lines can be used. For more information on mammalian cell culture, the following general references can be used: Culture of Animal Cells, Third Edition, edition by R. I. Freshney (Wiley-Liss, 1993); and Current Protocols in Molecular Biology, ed. By F. M. Ausubel, et al., (Greene Publishing Associates and Wiley & Sons, 1994), relevant portions incorporated herein by reference.

In another embodiment constructs can be generated which include a Hath1 or any atonal-associated nucleic acid sequence.

### EXAMPLE 18

### Maintenance of Packaging Cell Lines

The maintenance of packaging cell lines, such as the 293 and PT67 packaging cell lines, is described briefly. A vial of frozen cells is transferred from liquid N2 to a 37° C. water bath until just thawed. In order avoid osmotic shock to the cells, and to maximize cell survival, 1 ml of (Dulbecco's Modified Eagle Medium) DMEM is added to the tube and the mixture is transferred to a 15-ml tube. Another 5 ml of DMEM is added and the cells are mixed. After repeating these steps the final volume in the tube should be about 12 ml. Next, the cells are centrifuged at 500×g for 10 min. Finally, the supernatant is removed and the cells are resuspended in maintenance media as described in the next step. Generally, the cells are maintained in DMEM (high glucose: 4.5 g/L) containing 10% Fetal Bovine Serum (FBS), and 4 mM L-glutamine. If desired or necessary, 100 U/ml penicillin/100 g/ml streptomycin can be added. It is recommended that are plated at 3-5×10⁵ per 100-mm plate and split every 2 to 3 days, when they reach 70-80% confluency (confluence is 3-4×10⁶ per 100-mm plate). The PT67 cell line, for example, has a very short doubling time (<16 h) and should be split before they become confluent. The doubling time for EcoPack-293 cells is 24-36 h.

Cells are split be removing the medium and washing the cells once with PBS. After treatment with 1-2 ml of trypsin-EDTA solution for 0.5-1 min, 5 to 10 ml of media and serum is added to stop trypsinization. The cells are dispersed gently, but thoroughly, by pipetting and are resuspended. Alternatively, a predetermined portion of the cells is replated in a 100-mm plate in 10 ml of medium, followed by rotation or shaking of the plate to distribute the cells evenly. A ratio of up to 1:20 for the PT67 or EcoPack-293 cells is common.

Generally, the percentage of PT67 or EcoPack-293 cells capable of packaging retroviral vectors decreases slowly with continued passage of the cell line. Therefore, packaging cells should be reselected after 2 months of growth in culture. Alternatively, new high-titer cells can be purchased from, e.g., CLONTECH, or low passage number stocks can be frozen, stored and thawed to increase the viral yield.

### EXAMPLE 19

### Methods Utilizing a Retroviral Vector

The following protocol is used to transfect the retroviral vector for virus production, infection of target cells, and selection of stable clones. Other methods and vectors can also be used with the present invention to express Math1, such as those described in Retroviruses, ed. by J. M. Coffin & H. E. Varmus (1996, Cold Spring Harbor Laboratory Press, NY) and Current Protocols in Molecular Biology, ed. by F. M. Ausubel et al. (1994, Greene Publishing Associates and Wiley & Sons), incorporated herein by reference. In another embodiment these constructs include a Hath1 or any atonal-associated nucleic acid sequence.

Briefly, the transfection of the retroviral vector into PT67 cells was as follows. Math1 was cloned into pLNX as described hereinabove. The packaging cells were plated to a density of 5-7×10⁵ cells per 100-mm plate 12-24 hours before transfection. 1-2 hours before transfection, the medium replace with fresh medium. 25 M chloroquine can be added just prior to transfection. Chloroquine increases transfection efficiency 2-3 fold. A 25 mM stock solution of chloroquine can be made in distilled water and filter sterilized.

To each 100-mm plate 10-15 g of plasmid DNA using the desired method is transfected using, e.g., standard calcium-phosphate procedures (CalPhos Mammalian Transfection Kit, #K2050-1). The final volume of transfection mixture should not exceed 1 ml. The transfection solution is added to the medium and the plate is rotated to ensure even distribution. About 8 hours after transfection, a glycerol shock treatment can be performed to increase the uptake of DNA. After 10 to 24 hours post-transfection the medium was removed and the cells were washed twice with PBS, before adding 5 ml DMEM containing 10% FBS. The culture was incubated for an additional 12-48 hours to allow increase in virus titer. The virus titer reaches a maximum 48 hours post-transfection and is generally at least 30% of maximum between 24 and 72 hours post-transfection.

Alternatively, a stable virus-producing cell lines can also be selected. To obtain stable virus-producing cell lines, the transfected packaging cells are plated in a selection medium 2-3 days post-transfection. For G418 selection of neomycin resistance, the cells are selected in the presence of G418 (0.5 mg/ml “active”) for one week. Vectors carrying other selectable markers such as Puro, Bleo, or Hyg, can be used to obtain stable virus producing cell populations as well. Cell populations producing virions that produce titers of 105-106 recombinant virus particles per ml are common. Generally, 10⁵-10⁶ recombinant virus particles per ml is suitable for most purposes. For some studies, higher titer clones can be required. In this case, after antibiotic selection, individual clones are selected using, e.g., clone cylinders or limiting dilution, prior to propagation. Viral titer can be determined in a variety or ways, one such method is described hereinbelow. The viral titer produced by transiently transfected or stable virus-producing packaging cell lines is determined as follows, NIH/3T3 cells are plated one day prior to beginning the titer procedure. Cells are plated in 6-well plates at a density of 5×10⁴-1×10⁵ cells per well and 4 ml of media are added per well. Virus-containing medium is collected from packaging cells, and polybrene is added to a final concentration of 4 g/ml. The medium is filter-sterilized through a 0.45-m filter. Polybrene is a polycation that reduces the charge repulsion between the virus and the cellular membrane. The filter should be cellulose acetate or polysulfonic (low protein binding) but not nitrocellulose. Nitrocellulose binds proteins in the retroviral membrane, and consequently destroys the virus. Serial dilutions are prepared as follows: six 10-fold serial dilutions are usually sufficient. To dilute the virus, fresh medium containing 4 g/ml of polybrene is utilized. Next, NIH/3T3 target cells are infected by adding virus-containing medium to the wells. After 48 hours, the NIH/3T3 cells are stained. The titer of virus corresponds to the number of colonies present at the highest dilution that contains colonies, multiplied by the dilution factor. For example, the presence of four colonies in the 105 dilution would represent a viral titer of 4×10⁵.

For the infection of cells, the following procedure was followed. The target cells were plated 12-18 hours before infection at a cell density of 3-5×10⁵ per 100-mm plate. For the infection of cells that can be used for a biological assay, control cells can be treated with an insert-free virus produced under identical conditions. Half-maximal infection generally occurs after 5-6 hours of exposure of cells to virus, with maximal infection occurring after approximately 24 hours of exposure. The actual reverse transcription and integration of the retrovirus takes place within 24-36 hours of infection, depending on cell growth kinetics. Expression can be observed at 24 hours, and reaches a maximum at approximately 48 hours. Alternatively, infections can be conducted sequentially, about 12 hours apart. Sequential infection generally increases the efficiency of infection and also increases viral copy number. A minimum of 12 hours between each infection is recommended in order to ensure that cellular receptors will be unoccupied by viral envelope.

### EXAMPLE 20

### Screening Assays

Finally, the present invention also provides candidate substance screening methods that are based upon whole cell assays, in vivo analysis and transformed or immortal cell lines in which a reporter gene is employed to confer on its recombinant hosts a readily detectable phenotype that emerges only under conditions where Math1 would be expressed, is under-expressed or is over-expressed. Generally, reporter genes encode a polypeptide not otherwise produced by the host cell that is detectable by analysis, e.g., by chromogenic, fluorometric, radioisotopic or spectrophotometric analysis. In the present invention the Math1 gene has been replaced with P-galactosidase in a mouse.

An example of a screening assay of the present invention is presented herein. Math1 expressing cells are grown in microtiter wells, followed by addition of serial molar proportions of the small molecule candidate to a series of wells, and determination of the signal level after an incubation period that is sufficient to demonstrate, e.g., calretinin expression in controls incubated solely with the vehicle used to resuspend or dissolve the compound. The wells containing varying proportions of candidate are then evaluated for signal activation. Candidates that demonstrate dose related enhancement of reporter gene transcription or expression are then selected for further evaluation as clinical therapeutic agents. The stimulation of transcription can be observed in the absence of expressed Math1, in which case the candidate compound might be a positive stimulator of hair cell differentiation. Alternatively, the candidate compound might only give a stimulation in the presence of low levels of Math1, which would suggest that it functions to stabilize the formation of Math1 dimers or the interaction of Math1 with one or more transcriptional factors. Candidate compounds of either class might be useful therapeutic agents that would stimulate production of inner ear hair cells and thereby address the need of patients with hearing loss or balance control impairments.

### EXAMPLE 21

### Transfection of Cells with Math1 Retroviral Vectors

The present invention provides recombinant host cells transformed or transfected with a polynucleotide that encodes Math1, as well as transgenic cells derived from those transformed or transfected cells. In another embodiment these constructs include a Hath1 or any atonal-associated nucleic acid sequence. Preferably, a recombinant host cell of the present invention is transfected with a polynucleotide containing a functional Math1 nucleic acid sequence or a chimeric Math1 gene. Methods of transforming or transfecting cells with exogenous polynucleotides, such as DNA molecules, are well known in the art and include techniques such as calcium-phosphate- or DEAE-dextran-mediated transfection, protoplast fusion, electroporation, liposome mediated transfection, direct microinjection and adenovirus infection.

Math1 expression using recombinant constructs can be used to target the delivery of Math1 to cells in need thereof. Different promoter-vector combinations can be chosen by a person skilled in these arts to drive Math1 expression in different cell types. In some cases, the desired outcome can not be protein, but RNA, and recombinant vectors would include those with inserts present in either forward or reverse orientations. In addition, some vectors, for instance retroviruses or artificial recombination systems, can be designed to incorporate sequences within a cellular or viral genome in order to achieve constitutive or inducible expression of protein or RNA.

Many of the vectors and hosts are available commercially and have specific features that facilitate expression or subsequent purification. For instance DNA sequences to be expressed as proteins often appear as fusion with unrelated sequences that encode polyhistidine tags, or HA, FLAG, myc and other epitope tags for immunochemical purification and detection, or phosphorylation sites, or protease recognition sites, or additional protein domains such as glutathione S-transferase (GST), maltose binding protein (MBP) (New England Biolabs), and so forth that facilitate purification. Vectors can also be designed that contain elements for polyadenylation, splicing, and termination, such that incorporation of naturally occurring genomic DNA sequences that contain introns and exons can be produced and processed, or such that unrelated introns and other regulatory signals require RNA processing prior to production of mature, translatable RNAs. Proteins produced in the systems described above are subject to a variety of post-translational modifications, such as glycosylation, phosphorylation, nonspecific or specific proteolysis or processing.

### EXAMPLE 22

### Delivery of Math1 as an Amino Acid Sequence

A peptide (11 amino acids) derived from HIV has been recently described that when fused to full length proteins and injected into mice allow a rapid dispersal to the nucleus of all cells of the body (Schwarze et al., 1999). Schwarze et al. made fusion proteins to Tat ranging in size from 15 to 120 kDa. They documented a rapid uptake of the fusion proteins to the nuclei of cells throughout the animal, and the functional activity of said proteins was retained.

In an embodiment of the present invention there are constructs containing the Tat or Tat-HA nucleic acid sequence operatively linked to a Math1 nucleic acid sequence. In another embodiment these constructs include a Hath1 or any atonal-associated nucleic acid sequence. The vectors are expressed in bacterial cultures and the fusion protein is purified. This purified Tat-Math1 protein or Tat-Hath1 protein is injected into animal to determine the efficiency of the Tat delivery system into the inner ear, skin, cerebellum, brain stem, spinal cord and joints. Analysis is carried out to determine the potential of the Tat/Math1/Tat-Hath1 protein in hair cell and neuronal regeneration. This is a viable therapeutic approach either in its own right or in association with other methods or genes.

It should be understood that the methods to screen for compounds which affect Math1 expression disclosed herein are useful notwithstanding that effective candidates can not be found, since it is of practical utility to know what upstream effector is necessary for Math1 transcription.

### EXAMPLE 23

### Math1 is Required for Secretory Cell Lineage Commitment in Intestine

Two null alleles were utilized for Math1: Math1^{-/-} (with the coding region replaced by Hprt) and Math1^{β-Gal/β-Gal} (with the coding region replaced by the β-galactosidase gene, which is then expressed under the control of the Math1 promoter) (Ben-Arie et al., 2000). Math1 null mice die shortly after birth, but Math1 heterozygous mice survive to adulthood and appear normal. It was previously shown that Math1/LacZ expression faithfully mimics the endogenous gene expression (Ben-Arie et al., 2000). Herein Math1^{β-Gal/-} was used instead of Math1^{βGal/β-Gal} null mice for X-gal staining experiments to ensure equal copy numbers of the LacZ gene in heterozygous and null animals. Math1/LacZ expression within the gut is restricted to the intestinal epithelium starting at E16.5 and is sustained until adulthood.

X-gal staining of adult intestines was performed as described (Stappenbeck and Gordon, 2000); for embryos, 10-μm sections from frozen blocks of 4% paraformaldehyde-fixed intestinal tissue were stained overnight at 37° C. in a pH 8.0 solution containing 1.3 mM MgCl₂, 15 mM NaCl, 44 mM Hepes buffer (pH 7.3), 3 mM potassium ferri-cyanide, 3 mM potassium ferrocyanide and 0.05% X-gal. Sections were counterstained with nuclear fast red.

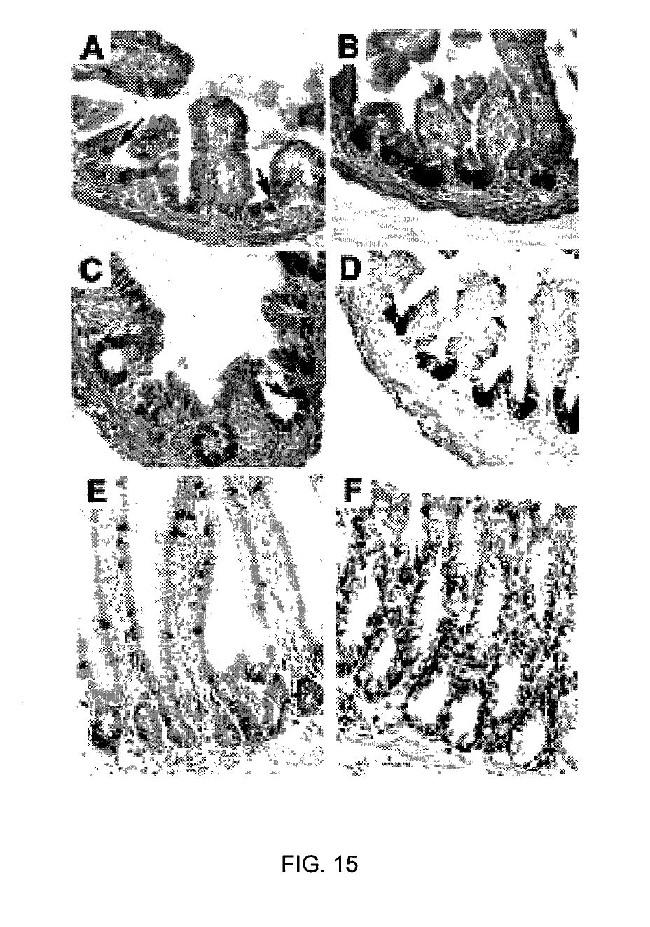

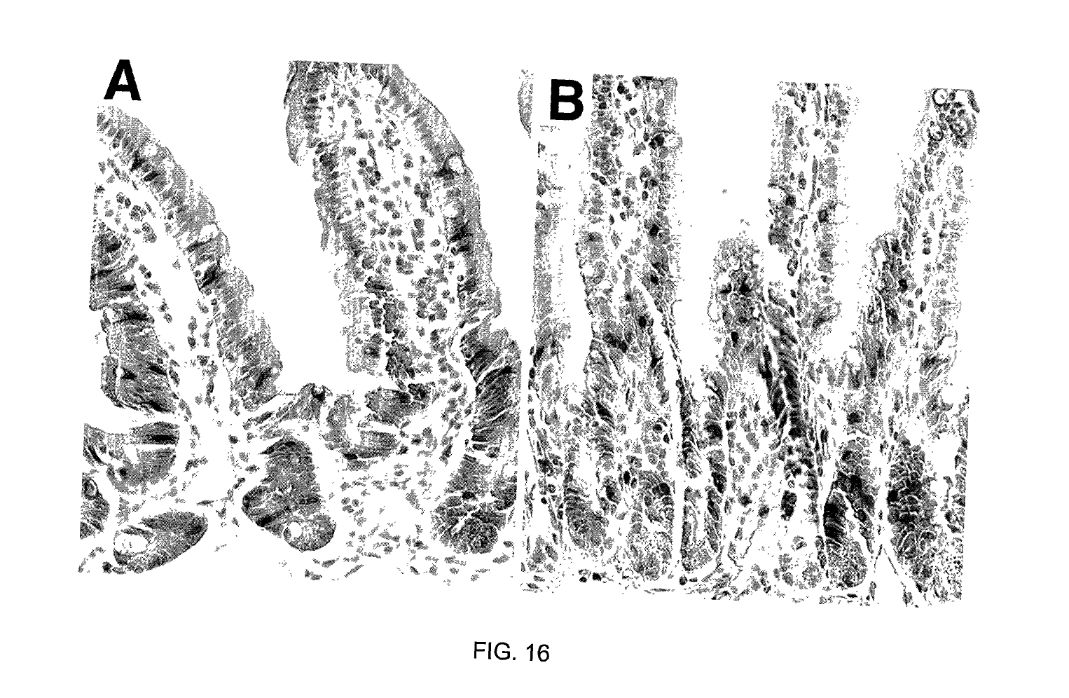

No Math1/LacZ expression was detected in the stomach, pancreas, or lung. In E18.5 heterozygous mice, LacZ-positive cells are sparsely scattered in the villi, the intervillus epithelium (FIG. 15A), and colonic crypts (FIG. 15C). In Math1 null littermates, however, LacZ-expressing cells are clustered in the intervillus region of ileum (FIG. 15B) and at the bases of the colonic crypts (FIG. 15D). Math1/LacZ expression persists throughout duodenum, jejunum, ileum, and colon (FIGS. 15, 15E and 15F; FIG. 16) in adult Math1^{β-Gal/β-Gal} mice. In the villi, the scattered blue cells appear to have a goblet cell morphology (a spherical vacuole); at the base of the crypt, most apical granule-containing Paneth cells appear to be LacZ-positive. X-gal stained cells are also found in the mid-crypt region. LacZ expression in adult crypts suggests that Math1 helps initiate cytodifferentiation of the epithelial cells. No Math1/LacZ expression was detected in the enteric nervous system (intestinal) from E14.5 to adult. An acetylcholinesterase activity assay (Blaugrund et al., 1996) revealed no gross abnormalities in the enteric neurons, although subtle deficits may not be apparent at these resolutions.

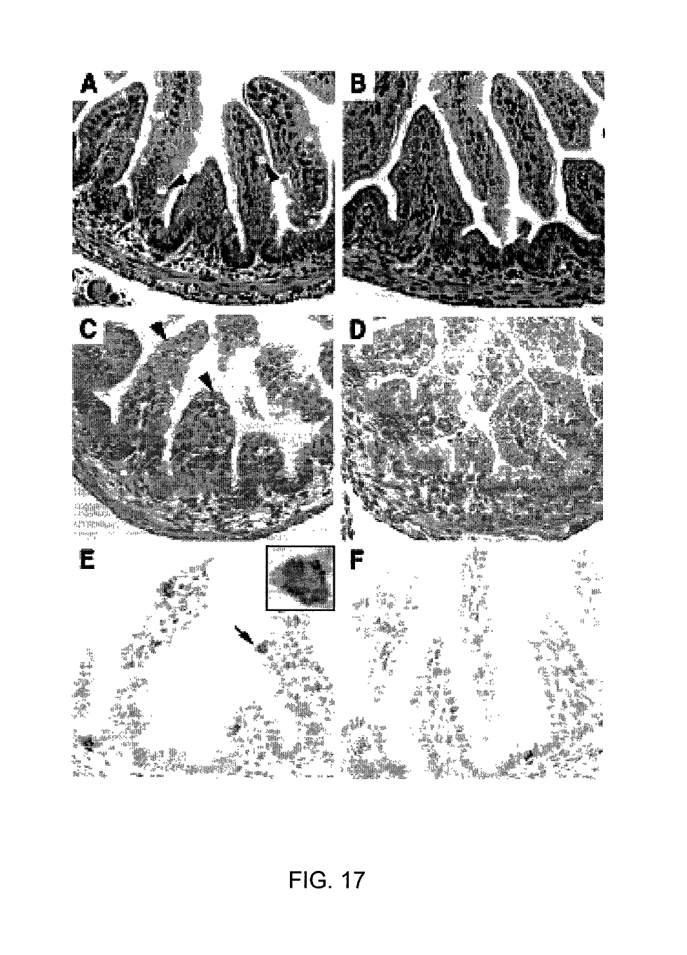

The small and large intestines of Math1 null embryos (E14.5 to E18.5) showed normal villus architecture, lamina propria, and musculature, but no goblet cells (FIGS. 17, A and B). In wild-type animals Alcian blue-positive goblet cells increased in number along the duodenal-colonal axis (FIG. 17C), but were not detected in Math1^{β-Gal/-} mice (FIG. 17D). The enteroendocrine lineage in the gut epithelium was then analyzed.

Hematoxylin and eosin or Alcian blue and neutral red staining and immunohistochemistry were performed according to standard protocols. The source and final dilution of the primary antibodies were as follows: rabbit chromogranin A antibody (1:2000), gastrin antibody (1:300), glucagon antibody (1:2000), serotonin antibody (1:20000), somatostatin antibody (1:4000), neurotensin antibody (1:2500) are from DiaSorin; rabbit synaptophysin antibody (1:200, Bio-Genex), and rabbit Ki-67 antibody (1:1000, Novocastra). For EM, different regions of E18.5 intestines were fixed in 3% phosphate-buffered glutaraldehyde and post fixed in phosphate-buffered osmium tetroxide. Specimens were dehydrated and embedded in Araldyte 502 resin. Semithin sections (0.4 μm) were stained with methylene blue and basic fuchsin. Thin sections (60 nM) were stained with uranyl acetate and lead citrate. The samples were observed on a JEOL 1210 electron microscope.

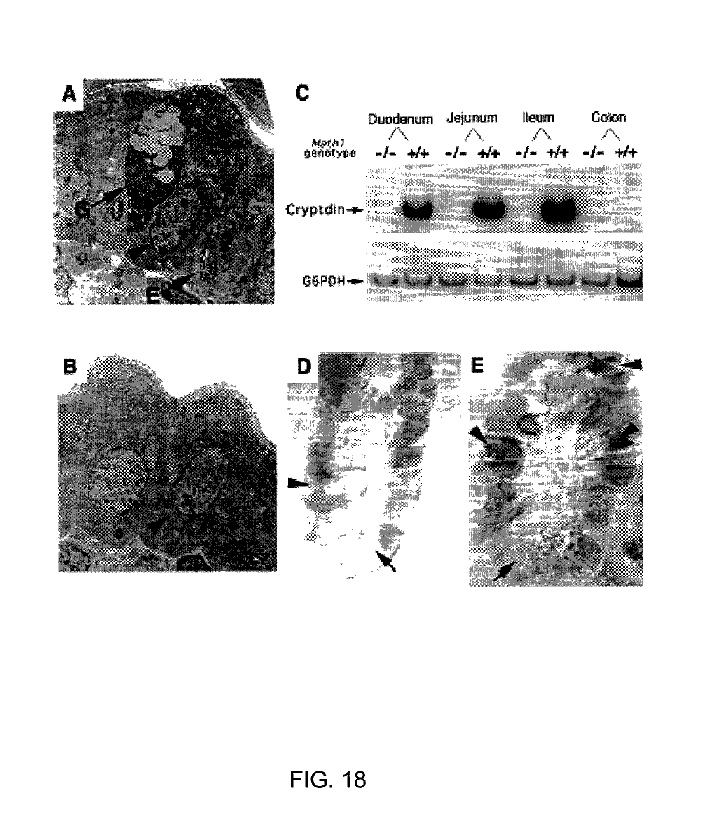

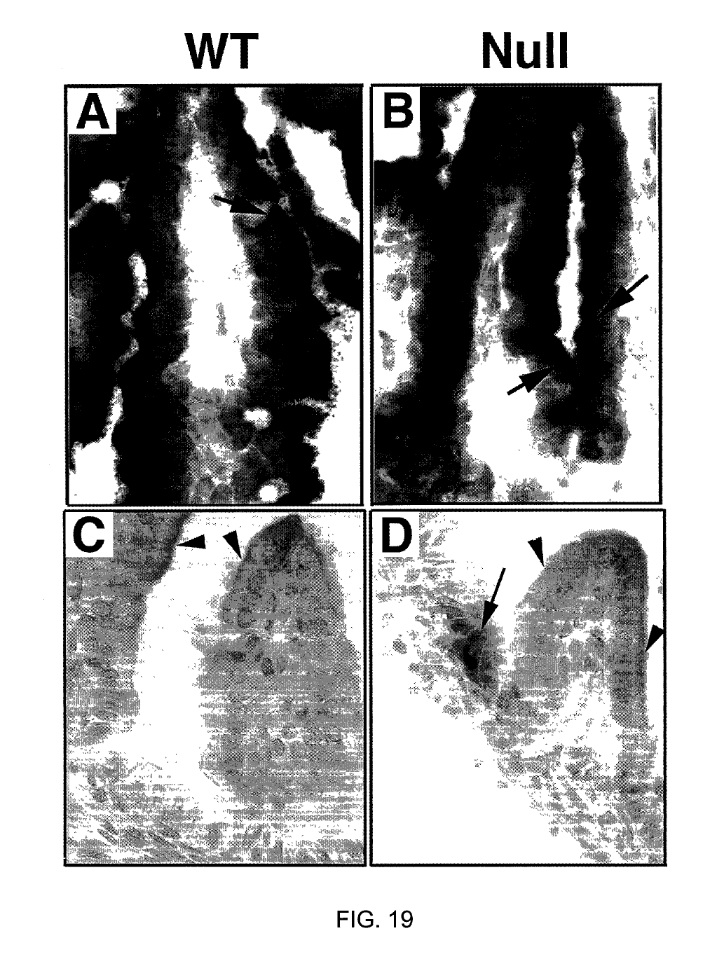

Neither panendocrine markers (chromogranin A, synaptophysin) nor specific endocrine markers (glucagon, gastrin, somatostatin, neurotensin, and serotonin) were detectable in any regions of Math1^{β-Ga;/-} null mouse intestine (FIG. 17F; cf. wild type, FIG. 17E). Electron microscopy (EM) on E18.5 embryos revealed no granular or common goblet or enteroendocrine cells in any region of Math1^{β-Gal/-} null mouse intestines (FIG. 18B, cf. wild type in 18A). Null mouse enterocytes, however, had a normal microvillus brush border: strongly positive for alkaline phosphatase and lactase, ample endoplasmic reticulum, a few secondary lysosomes, and regular columnar height with uniform nuclei close to the inner aspect of the cell (FIG. 18B; FIG. 19). Some mutant enterocytes have abundant glycogen, like immature enterocytes, whereas wild-type enterocytes no longer have cytoplasmic clusters. Electron microscopy cannot be used to evaluate Paneth cells in Math1 null animals, because their characteristic apical granules do not mature until after birth (Stappenbeck and Gordon, 2000). But cryptdin-1 is one of the earliest markers expressed in Paneth cells, starting at E15.5 (Bry et al., 1994), so its expression was examined.

RNA was extracted from E18.5 intestine using TRIzol (Gibco BRL) according to manufacturer's instructions. cDNA synthesis was performed as described (Jensen et al., 1998). Cryptdin and glucose-6-phosphate dehydrogenase (G6PDH) primers and PCR were as previously described (Darmoul et al., 1997; Jensen et al., 2000), except for the thermal cycle pro file: a single denaturing step at 96° C. for 1 min followed by 25 cycles of 96° C. for 30 s; 55° C. for 30 s;73° C. for 1 min.

Cryptdin-1 consensus primers were used to amplify a 272-bp product corresponding to nucleotides 80 to 352 (Darmoul et al., 1997). Cryptdin-1 expression was detected in wild-type duodenum, jejunum, and ileum but was completely absent in these three regions in Math1 null animals (FIG. 18C). As expected (Bry et al., 1994), no cryptdin-positive cells were detected in wild-type or Math1 null colon (FIG. 18C). Neither EM nor tunnel assays revealed signs of premature cell death in Math1 null gut (FIG. 18B).

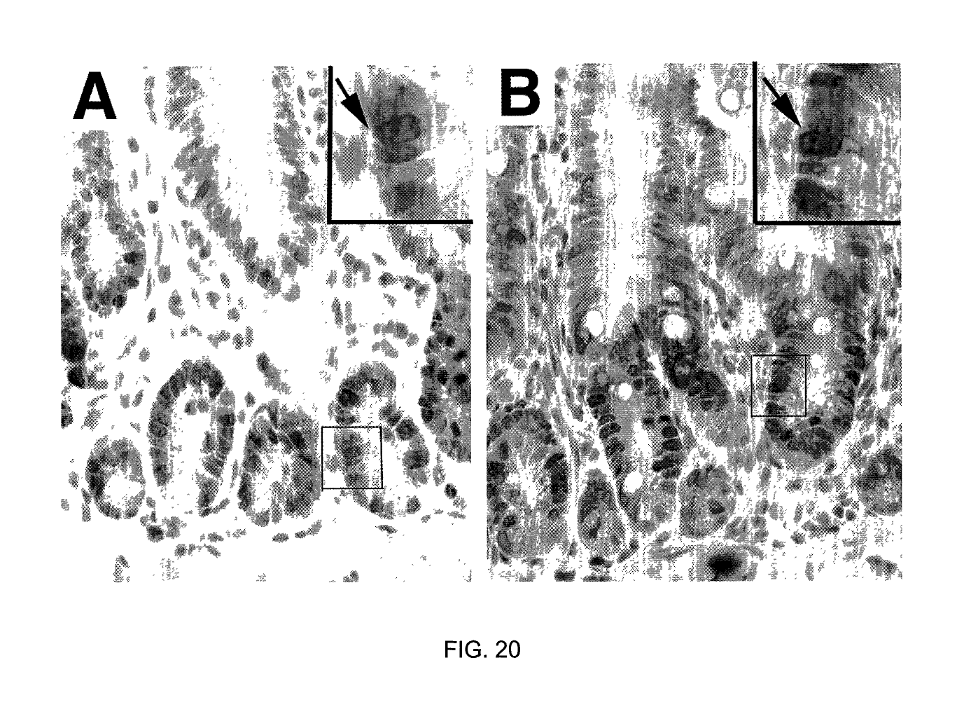

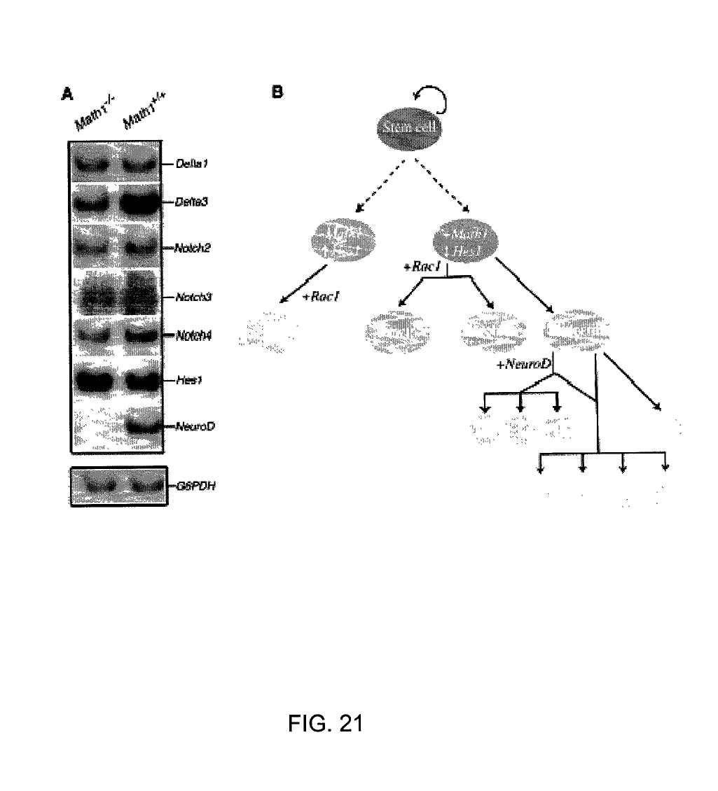

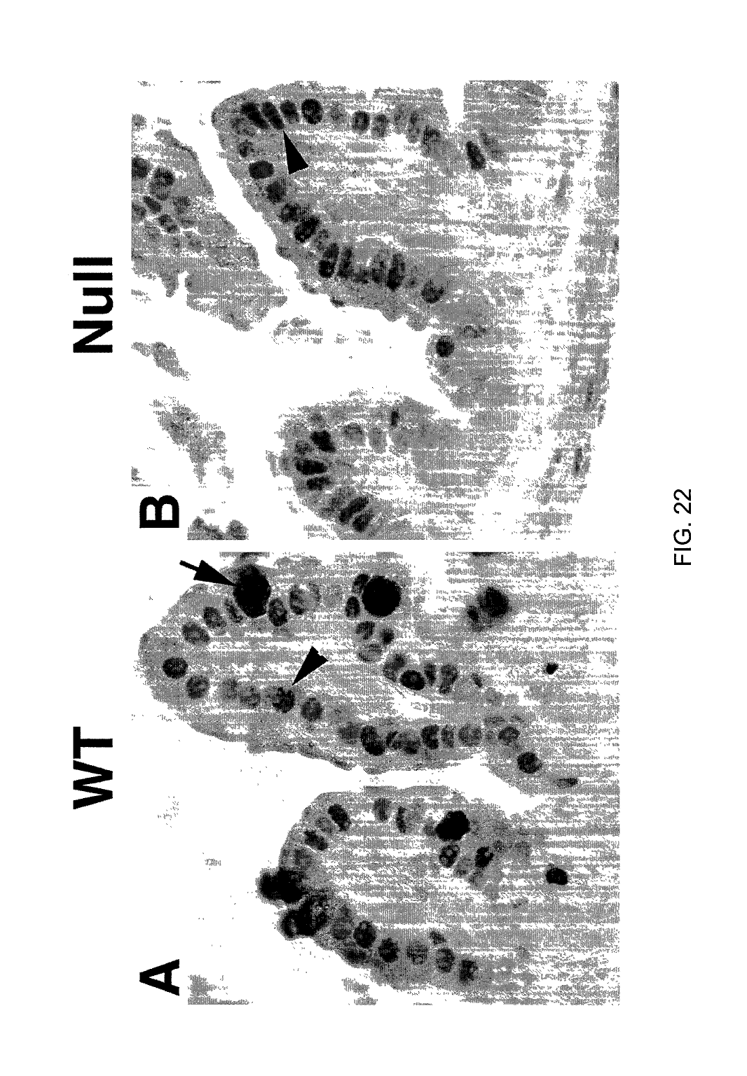

In adult crypts, epithelial stem cells and multipotent progenitor cells are proliferating and show nuclear staining for Ki-67, a cell proliferation marker (Korinek et al., 1998) (FIGS. 18D and 18E). In the Math1^{β-Gal/β} mice, the LacZ-expressing cells show a cytoplasmic blue staining pattern (Ben-Arie et al., 2000). This feature permits colocalization of Math1/β-galactosidase and Ki-67. In the crypts, double-positive cells are scattered from the 4th to 13th cell position from the base of the small intestine and are in the 2nd to 4th position in the colon (FIGS. 18D and 18E; FIG. 20). Clearly not all Ki-67-positive cells express Math1, suggesting that Math1-negative progenitors give rise to the entero-cytes, whereas Math1-expressing progenitors become goblet, enteroendocrine, and Paneth cells. Upon deletion of Math1, the latter group of cells fail to differentiate, and their progenitors remain in the proliferating stage, thus accounting for the intense X-gal staining seen in the crypts of null embryos (FIGS. 15B and 15D). To ascertain the effects of Math1 deletion on the proliferative status of the secretory lineage progenitors, 2500 Ki-67-positive cells were examined in three pairs of E18.5 Math1 null and heterozygous mice for LacZ-positive staining. Double-positive cells were scored as a fraction of the total cycling Ki-67-positive population in E18.5 Math1 heterozygous and null mouse intestines. The ratio of double-positive to Ki-67-positive cells in Math1 null animals, from duodenum to colon, was roughly three times that seen in heterozygotes (25 to 68% versus 7 to 22%), supporting the hypothesis that cells lacking Math1 fail to exit the cell cycle and differentiate. Previous studies have shown that members of the Notch signaling pathway (e.g., Mashl, Neurogenin 3, and NeuroD) are involved in endocrine cell differentiation (Ito et al., 2000; Apelqvist et al., 1999; Rind1 et al., 1999). Deletion of Hes1, a Notch signaling component that represses bHLH transcriptional activators, leads to an increased number of enteroendocrine and goblet cells but fewer enterocytes, and elevates expression of Delta1, Delta3, NeuroD, and Math1 in the small intestine (Jensen et al., 2000). Hesl also negatively regulates inner ear hair cell differentiation by suppressing Math1 (Zheng et al., 2000). These studies support the hypothesis that Math1 controls cell fate determination via a Delta-Notch signaling pathway. Quantitative reverse transcription-poly-merase chain reaction (RT-PCR) analysis revealed that Delta3 was reduced to half of wild-type levels in Math1 null mice, and NeuroD expression was lost completely (FIG. 21A). In contrast, Deltal, Hes-1, and Notch1, 2, 3, and 4 expression levels and cellular localization of Hes-1 appeared unaffected (FIG. 21A and FIG. 22). These observations are consistent with previous findings that Math1 is upstream of NeuroD (Miyata et al., 1999) and the notion that Math1 has a positive feedback effect on Notch ligand (e.g., Delta3) expression. These findings provide new insight into the role of Notch-mediated lateral inhibition in controlling differentiation of intestinal epithelial lineages. Building on the model set forth by Bjerknes and Cheng (1999), in a specific embodiment a single self-maintaining stem cell gives rise to two daughter cells directly or through intermediate progenitors (FIG. 21B). In one daughter cell, interaction between Delta and Notch homologs elevates Hes1 expression, inhibiting Math1 expression, and this cell adopts an enterocyte fate. In the other daughter cell, lack of Hesl expression increases Math1 expression, and this cell becomes a committed multipotent progenitor that will differentiate into a secretory lineage cell (FIG. 21B). Further differentiation of the secretory lineage into goblet, enteroendocrine, and Paneth cells requires other factors. NeuroD has been shown to play a role in differentiation of the secretin and cholecystokinin enteroendocrine cells (Rind1 et al., 1999); early committed multipotential endocrine cells can branch into at least three lineages (FIG. 21B) (Rind1 et al., 1999). Rac1 is reported to play a positive role in goblet and Paneth cell differentiation but does not seem to have any impact on the enteroendocrine lineage (Stappenbeck and Gordon, 2000), suggesting that goblet and Paneth cells share a closer relationship during later development. Constitutively activated Rac1 causes precocious enterocyte growth, indicating its positive role in the absorptive cell lineage (Stappenbeck and Gordon, 2000). These observations suggest that there is cross talk between the Notch and Rho GTPase pathways during formnation of the gut epithelium. In other specific embodiments, instead of arising from one Math1-positive progenitor, the goblet, enteroendocrine, and Paneth cells may differentiate from three distinct progenitors that each express Math1.

References

All patents and publications are herein incorporated by reference to the same extent as if each individual publication was specifically and individually indicated to be incorporated by reference.

Publications

Akazawa, C., Ishibashi, M., Shimizu, C., Nakanishi, S., and Kageyama, R. (1995). A mammalian helix-loop-helix factor structurally related to the product of Drosophila proneural gene atonal is a positive transcriptional regulator expressed in the developing nervous system. J Biol Chem 270, 8730-8.

Alder, J., Cho, N., and Hatten, M. (1996). Embryonic precursor cells from the rhombic lip are specified to a cerebellar granule neuron identity. Neuron 17, 389-399.

Altman, J., and Bayer, S. A. (1996). Development of the Cerebellar System: In Relation to its Evolution, Structure, and Functions. Boca Raton, Fla.: CRC Press.

Anderson, D. J. (1995). Neural development. Spinning skin into neurons. Curr Biol 5, 1235-8.

Apelqvist, A. et al., Nature 400, 877 (1999).

Bach, S. P., A. G. Renehan, C. S. Potten, Carcinogenesis 21, 469 (2000).

Bartholomä, A., and Nave, K. A. (1994). NEX-1: a novel brain-specific helix-loop-helix protein with autoregulation and sustained expression in mature cortical neurons. Mech Dev 48, 217-28.

Beck, F., F. Tata, K. Chawengsaksophak, Bioessays 22, 431 (2000).

Ben-Arie, N., Hassan, B. A., Birmingham, N. A., Malicki, D. M., Armstrong, D., Matzuk, M., Bellen, H. J., and Zoghbi, H. Y. (2000). Functional conservation of atonal and Math1 in the CNS and PNS. Development 127: 1039-1048.

Ben-Arie, N., Bellen, H. J., Armstrong, D. L., McCall, A. E., Gordadze, P. R., Guo, Q., Matzuk, M. M., and Zoghbi, H. Y. (1997). Math1 is essential for genesis of cerebellar granule neurons. Nature 390, 169-172.

Ben-Arie, N., McCall, A. E., Berkman, S., Eichele, G., Bellen, H. J., and Zoghbi, H. Y. (1996). Evolutionary conservation of sequence and expression of the bHLH protein Atonal suggests a conserved role in neurogenesis. Human Molecular Genetics 5, 1207-1216.

Bermingham, N. A., Hassan, B. A., Price, S. D., Vollrath, M. A., Ben-Arie, N., Eatock, R. A., Bellen, H. J., Lysakowski, A., and Zoghbi, H. Y. (1999). Math1: An essential gene for the generation of inner ear hair cells. Science 284, 1837-41.

Bi, W., Deng, J. M., Zhang, Z., Behringer, R. R., and de Crombrugghe, B. (1999). Sox9 is required for cartilage formation. Nat Genet 22, 85-9.

Bjerknes, M., H. Cheng, Gastroenterology 116, 7 (1999).

Blaugrund, E. et al., Development 122 ,309 (1996).

Bonnerot, C., and Nicolas, J. F. (1993). Application of LacZ gene fusions to postimplantation development. Methods Enzymol 225, 451-69.

Boyan, G. S. (1993). Another look at insect audition: the tympanic receptors as an evolutionary specialization of the chordotonal system. J Insect Physiol 39, 187-200.

Brand, A. H., and Perrimon, N. (1993). Targeted gene expression as a means of altering cell fates and generating dominant phenotypes. Development 118, 401-15.

Brown, N. L., Kanekar, S., Vetter, M. L., Tucker, P. K., Gemza, D. L., and Glaser, T. (1998). Math5 encodes a murine basic helix-loop-helix transcription factor expressed during early stages of retinal neurogenesis. Development 125, 4821-4833.

Bry et al., Proc.Natl.Acad.Sci.U.S.A.91 ,10335 (1994).

Buckwalter, J. A., and Mankin, H. J. (1998). Articular cartilage: tissue design and chondrocyte-matrix interactions. Instr Course Lect 47, 477-86.

Cheng, C. P.Leblond, Am.J.Anat.141, 537 (1974).

Chien, C. T., Hsiao, C. D., Jan, L. Y., and Jan, Y. N. (1996). Neuronal type information encoded in the basic-helix-loop-helix domain of proneural genes. Proc Natl Acad Sci USA 93, 13239-44.

Clatworthy, J. P., V.Subramanian, Mech.Dev.101, 3 (2001).

Darmoul, D. D. Brown, M. E. Selsted, A. J. Ouellette, Am.J.Physiol.272,G197 (1997).

Davis, R. L., Cheng, P. F., Lassar, A. B., and Weintraub, H. (1990). The MyoD DNA binding domain contains a recognition code for muscle-specific gene activation. Cell 60, 733-46.

Dreller, C., and Kirschner, W. H. (1993). Hearing in honeybees: localization of the audiotory sense organ. J. Comp Physio A 173, 275-279.

Eberl, D. F. (1999). Feeling the vibes: chordotonal mechanisms in insect hearing. Curr Opin Neurobiol 9, 389-393.

Ferguson, B. M., Brockdorff, N., Formistone, E., Ngyuen, T., Kronmiller, J. E., and Zonana, J. (1997). Cloning of Tabby, the murine homolog of the human EDA gene: evidence for a membrane-associated protein with a short collagenous domain. Hum Mol Genet 6, 1589-94.

Fode, C., Gradwohl, G., Morin, X., Dierich, A., LeMeur, M., Goridis, C., and Guillemot, F. (1998). The bHLH protein NEUROGENIN 2 is a determination factor for epibranchial placode-derived sensory neurons. Neuron 20, 483-94.

Friedrich, G., and Soriano, P. (1991). Promoter traps in embryonic stem cells: a genetic screen to identify and mutate developmental genes in mice. Genes Dev 5, 1513-23.

Ghysen, A., and Dambly-Chaudiere, C. (1989). Genesis of the Drosophila peripheral nervous system. Trends Genet 5, 251-5.

Gordon, J. I., G. H. Schmidt, K. A. Roth, FASEB J.6, 3039 (1992).

Gruneberg, H. (1971). The tabby syndrome in the mouse. Proc R Soc Lond B Biol Sci 179, 139-156.

Guillemot, F. (1995). Analysis of the role of basic-helix-loop-helix transcription factors in the development of neural lineages in the mouse. Biol Cell 84, 227-241.

Hatten, M. E., and Heintz, N. (1995). Mechanisms of neural patterning and specification in the developing cerebellum. Ann Rev Neurosci 18, 385-408.

Helms, A. W., and Johnson, J. E. (1998). Progenitors of dorsal commissural interneurons are defined by MATH1 expression. Development 125, 919-28.

Horton, W. A., Machado, M. A., Ellard, J., Campbell, D., Putnam, E. A., Aulthouse, A. L., Sun, X., and Sandell, L. J. (1993). An experimental model of human chondrocyte differentiation. Prog Clin Biol Res 383B, 533-40.

Ito, T. et al., Development 127, 3913 (2000).

Jarman, A. P., and Ahmed, I. (1998). The specificity of proneural genes in determining Drosophila sense organ identity. Mech Dev 76, 117-25.

Jarman, A. P., Grau, Y., Jan, L. Y., and Jan, Y. N. (1993). atonal is a proneural gene that directs chordotonal organ formation in the Drosophila peripheral nervous system. Cell 73, 1307-21.

Jensen, J., P. Serup, C. Karlsen, T. F. Nielsen, O. D. Madsen, J.Biol.Chem. 271, 18749 (1996).

Jensen, J. et al., Nature Genet. 24 ,36 (2000).

Kaestner, K. H., D. G. Silberg, P. G. Traber, G. Schutz, Genes Dev.11 ,1583 (1997).

Kania, A., Salzberg, A., Bhat, M., D'Evelyn, D., He, Y., Kiss, I., and Bellen, H. J. (1995). P-element mutations affecting embryonic peripheral nervous system development in Drosophila melanogaster. Genetics 139, 1663-1678.

Karsenty, G. (1998). Genetics of skeletogenesis. Dev Genet 22, 301-13.

Korinek, V. et al., Nature Genet.19, 379 (1998).

Lee, J. E. (1997). Basic helix-loop-helix genes in neural development. Curr. Opin. Neurobiol 7, 13-20.

Lee, K. J., Mendelsohn, M., and Jessell, T. M. (1998). Neuronal patterning by BMPs: a requirement for GDF7 in the generation of a discrete class of commissural interneurons in the mouse spinal cord. Genes Dev 12, 3394-407.

Liu, X. Y., Dangel, A. W., Kelley, R. I., Zhao, W., Denny, P., Botcherby, M., Cattanach, B., Peters, J., Hunsicker, P. R., Mallon, A. M., Strivens, M. A., Bate, R., Miller, W., Rhodes, M., Brown, S. D., and Herman, G. E. (1999). The gene mutated in bare patches and striated mice encodes a novel 3beta-hydroxysteroid dehydrogenase. Nat Genet 22, 182-7.

Ma, Q., Chen, Z., del Barco Barrantes, I., de la Pompa, J. L., and Anderson, D. J. (1998). neurogenin1 is essential for the determination of neuronal precursors for proximal cranial sensory ganglia. Neuron 20, 469-82.

McCormick, M. B., Tamimi, R. M., Snider, L., Asakura, A., Bergstrom, D., and Tapscott, S. J. (1996). NeuroD2 and neuroD3: distinct expression patterns and transcriptional activation potentials within the neuroD gene family. Mol Cell Biol 16, 5792-800.

McIver, S. B. (1985). Mechanoreception. In Comprehensive Insect Physiology, Biochemistry, and Pharmacology (G. A. Kerkut and L. I. Gilbert, Eds.), pp. 71-132. Oxford: Pergamon Press.

Miyata, T., T. Maeda, J. E. Lee, Genes Dev.13 ,1647 (1999).

Moulins, M. (1976). Ultrastructure of chordotonal organs. In Structure and Function of Proprioceptors in the Invertebrates (P. J. Mill, Ed.), pp. 387-426. London: Chapman and Hall.

Munger, B. L. (1991). The Biology of Merkel Cells. In Physiology, Biochemistry, and Molecular Biology of the Skin, second edition (L. A. Goldsmith, Ed.), pp. 836-856. Oxford, UK: Oxford University Press.

Pabst, O., R. Zweigerdt, H. H. Arnold,Development 126, 2215 (1999).

Pinkus, F. (1905). Über Hautsinnesorgane neben dem menschlichen Haar (Haarscheiben) und ihre verglei chend-anatomische Bedeutung. Arch mikr Anat 65, 121-179.

Poole, C. A. (1997). Articular cartilage chondrons: form, function and failure. J Anat 191, 1-13.

Rindi, G. et al., Development 126, 4149 (1999).

Rodriguez, I., Hernandez, R., Modolell, J., and Ruiz-Gomez, M. (1990). Competence to develop sensory organs is temporally and spatially regulated in Drosophila epidermal primordia. Embo J 9, 3583-92.

Sambrook, Fritsch, Maniatis, In: Molecular Cloning: A Laboratory Manual, Vol. 1, Cold Spring Harbor Laboratory Press, Cold Spring Harbor, N.Y., Ch. 7,7.19-17.29, 1989.

Schwarze, S. R., Ho, A., Vocero-Akbani, A. and Dowdy, S. F. (1999). In vivo protein transduction: delivery of a biologically active protein in the mouse. Science 285, 1569-72.

Shimizu, C., Akazawa, C., Nakanishi, S., and Kageyama, R. (1995). MATH-2, a mammalian helix-loop-helix factor structurally related to the product of Drosophila proneural gene atonal, is specifically expressed in the nervous system. Eur J Biochem 229, 239-48.

Stappenbeck, T. S. and J. I. Gordon,Development 127, 2629 (2000).

Sun, Y., Jan, L. Y., and Jan, Y. N. (1998). Transcriptional regulation of atonal during development of the Drosophila peripheral nervous system. Development 125, 3731-40.

Takebayashi, K., Takahashi, S., Yokota, C., Tsuda, H., Nakanishi, S., Asashima, M., and Kageyama, R. (1997). Conversion of ectoderm into a neural fate by ATH-3, a vertebrate basic helix-loop-helix gene homologous to Drosophila proneural gene atonal. Embo J 16, 384-95.

Tautz, D., and Pfeifle, C. (1989). A nonradioactive in situ hybridization method for the localization of specific RNAs in Drosophila embryos reveals translation control of the segmentation gene hunchback. Chromosoma 98, 81-85.

Vaessin, H., Caudy, M., Bier, E., Jan, L. Y., and Jan, Y. N. (1990). Role of helix-loop-helix proteins in Drosophila neurogenesis. Cold Spring Harb Symp Quant Biol 55, 239-45.

van Staaden, M. J., and Römer, H. (1998). Evolutionary transition from stretch to hearing organs in ancient grasshoppers. Nature 384, 773-776.

Vielkind, U., Sebzda, M. K., Gibson, I. R., and Hardy, M. H. (1995). Dynamics of Merkel cell patterns in developing hair follicles in the dorsal skin of mice, demonstrated by a monoclonal antibody to mouse keratin 18. Acta Anat 152, 93-109.

Yamakado, M., and Yohro, T. (1979). Subdivision of mouse vibrissae on an embryological basis, with descriptions of variations in the number and arrangement of sinus hairs and cortical barrels in BALB/c (nu/+; nude, nu/nu) and hairless (hr/hr) strains. Am J Anat 155, 153-173.

Zhao, C., and Emmons, S. W. (1995). A transcription factor controlling development of peripheral sense organs in C. elegans. Nature 373, 74-78.

Zheng, J. L., J. Shou, F. Guillemot, R. Kageyama, W. Gao, Development 127, 4551 (2000).

Patents

U.S. Pat. No. 5,840,873, issued Nov. 24, 1998

U.S. Pat. No. 5,843,640, issued Dec. 1, 1998

U.S. Pat. No. 5,843,650, issued Dec. 1, 1998

U.S. Pat. No. 5,843,651, issued Dec. 1, 1998

U.S. Pat. No. 5,843,663, issued Dec. 1, 1998

U.S. Pat. No. 5,846,708, issued Dec. 8, 1998

U.S. Pat. No. 5,846,709, issued Dec. 8, 1998

U.S. Pat. No. 5,846,717, issued Dec. 8, 1998

U.S. Pat. No. 5,846,726, issued Dec. 8, 1998

U.S. Pat. No. 5,846,729, issued Dec. 8, 1998

U.S. Pat. No. 5,846,783, issued Dec. 8, 1998

U.S. Pat. No. 5,849,481, issued Dec. 15, 1998

U.S. Pat. No. 5,849,483, issued Dec. 15, 1998

U.S. Pat. No. 5,849,486, issued Dec. 15, 1998

U.S. Pat. No. 5,849,487, issued Dec. 15, 1998

U.S. Pat. No. 5,849,497, issued Dec. 15, 1998

U.S. Pat. No. 5,849,546, issued Dec. 15, 1998

U.S. Pat. No. 5,849,547, issued Dec. 15, 1998

U.S. Pat. No. 5,851,770, issued Dec. 22, 1998

U.S. Pat. No. 5,851,772, issued Dec. 22, 1988

U.S. Pat. No. 5,853,990, issued Dec. 29, 1998

U.S. Pat. No. 5,853,993, issued Dec. 29, 1998

U.S. Pat. No. 5,853,992, issued Dec. 29, 1998

U.S. Pat. No. 5,856,092, issued Jan. 5, 1999

U.S. Pat. No. 5,858,652, issued Jan. 12, 1999

U.S. Pat. No. 5,861,244, issued Jan. 19, 1999

U.S. Pat. No. 5,863,732, issued Jan. 26, 1999

U.S. Pat. No. 5,863,753, issued Jan. 26, 1999

U.S. Pat. No. 5,866,331, issued Feb. 2, 1999

U.S. Pat. No. 5,866,336, issued Feb. 2, 1999

U.S. Pat. No. 5,866,337, issued Feb. 2, 1999

U.S. Pat. No. 5,900,481, issued May 4, 1999

U.S. Pat. No. 5,905,024, issued May 18, 1999

U.S. Pat. No. 5,910,407, issued Jun. 8, 1999

U.S. Pat. No. 5,912,124, issued Jun. 15, 1999

U.S. Pat. No. 5,912,145, issued Jun. 15, 1999

U.S. Pat. No. 5,912,148, issued Jun. 15, 1999

U.S. Pat. No. 5,916,776, issued Jun. 29, 1999

U.S. Pat. No. 5,916,779, issued Jun. 29, 1999

U.S. Pat. No. 5,919,626, issued Jul. 6, 1999

U.S. Pat. No. 5,919,630, issued Jul. 6, 1999

U.S. Pat. No. 5,922,574, issued Jul. 13, 1999

U.S. Pat. No. 5,925,517, issued Jul. 20, 1999

U.S. Pat. No. 5,925,525, issued Jul. 20, 1999

U.S. Pat. No. 5,928,862, issued Jul. 27, 1999

U.S. Pat. No. 5,928,869, issued Jul. 27, 1999

U.S. Pat. No. 5,928,870, issued, Jul. 27, 1999

U.S. Pat. No. 5,928,905, issued Jul. 27, 1999

U.S. Pat. No. 5,928,906, issued Jul. 27, 1999

U.S. Pat. No. 5,929,227, issued Jul. 27, 1999

U.S. Pat. No. 5,932,413, issued Aug. 3, 1999

U.S. Pat. No. 5,932,451, issued Aug. 3, 1999

U.S. Pat. No. 5,935,791, issued Aug. 10, 1999

U.S. Pat. No. 5,935,825, issued Aug. 10, 1999

U.S. Pat. No. 5,939,291, issued Aug. 17,1999

U.S. Pat. No. 5,942,391, issued Aug. 24, 1999

European Application No. 320 308

European Application No. 329 822

GB Application No. 2 202 328

PCT Application No. PCT/US87/00880

PCT Application No. PCT/US89/01025

PCT Application WO 88/10315

PCT Application WO 89/06700

PCT Application WO 90/07641

One skilled in the art readily appreciates that the present invention is well adapted to carry out the objectives and obtain the ends and advantages mentioned as well as those inherent therein. Sequences, mutations, complexes, methods, treatments, pharmaceutical compositions, procedures and techniques described herein are presently representative of the preferred embodiments and are intended to be exemplary and are not intended as limitations of the scope. Changes therein and other uses will occur to those skilled in the art which are encompassed within the spirit of the invention or defined by the scope of the pending claims.

What is claimed is:

1. A method of predicting a differentiation state for a stem cell, comprising the steps of:
obtaining the cell;
determining the expression status of an atonal-associated sequence.

2. The method of claim 1, wherein said stem cell is an intestinal stem cell.

3. The method of claim 2, wherein the stem cell is obtained from an intestinal epithelium.

4. The method of claim 1, wherein said expression status of said atonal-associated sequence is an upregulation of expression of said atonal-associated sequence.

5. The method of claim 4, wherein said differentiation state is to a secretory cell of the intestine.

6. The method of claim 5, wherein said secretory cell is at least one of a goblet cell, an enteroendocrine cell, or a Paneth cell.

7. The method of claim 1, wherein said expression status of said atonal-associated sequence is a downregulation of expression of said atonal-associated sequence.

8. The method of claim 7, wherein said differentiation state is to an absorptive cell of the intestine.

9. The method of claim 1, wherein said atonal-associated sequence is a polynucleotide.

10. The method of claim 1, wherein said atonal-associated sequence is a polypeptide.

11. A method for differentiating a stem cell, comprising altering expression of an atonal-associated sequence.

12. The method of claim 11, wherein said stem cell is a gastrointestinal stem cell.

13. The method of claim 11, wherein said stem cell differentiates into a secretory cell.

14. The method of claim 13, wherein said secretory cell is at least one of a goblet cell, an enteroendocrine cell, or a Paneth cell.

15. The method of claim 11, wherein said stem cell differentiates into an absorptive cell.

16. A method of regenerating secretory intestinal cells in an individual, comprising the step of administering to the individual a stem cell and a regulatory factor for said stem cell, wherein the expression of an atonal-associated sequence is upregulated in the stem cell.

17. The method of claim 16, wherein the secretory intestinal cell is at least one of a goblet cell, an enteroendocrine cell, or a Paneth cell.

18. The method of claim 16, wherein the regulatory factor is a bone morphogenetic protein.

19. The method of claim 18, wherein the bone morphogenetic protein is GDF7.

20. A method of regenerating absorptive intestinal cells in an individual, comprising the step of administering to the individual a stem cell and a regulatory factor for said stem cell, wherein the expression of an atonal-associated sequence is downregulated in the stem cell.

21. The method of claim 20, wherein the regulatory factor is a member of the HES family.

22. The method of claim 21, wherein the HES family member is Hes1, Hes2, Hes3, Hes4, Hes5, Hes6, Hes7, HERP1 or HERP2.

23. A method of treating an animal for a gastrointestinal condition, comprising delivering to the animal a gastrointestinal stem cell.

24. The method of claim 23, wherein the method further comprises delivery of a regulatory factor.

25. A method of treating an animal for a gastrointestinal condition comprising delivering a therapeutically effective amount of an atonal-associated amino acid sequence or nucleic acid sequence to a cell of said animal.

26. The method of claim 25, wherein said gastrointestinal condition is cancer, damaged intestinal tissue, inflammatory bowel disease, irritable bowel syndrome, infection or necrotizing entercolitis.

27. The method of claim 25, wherein said atonal-associated amino acid sequence or nucleic acid sequence is Math1.

28. The method of claim 25, wherein said atonal-associated amino acid sequence or nucleic acid sequence is Hath1.

29. The method of claim 25 wherein said amino acid sequence or nucleic acid sequence is administered by a delivery vehicle.

30. The method of claim 29 wherein said delivery vehicle is an adenoviral vector, a retroviral vector, an adeno-associated viral vector, a plasmid, a liposome, a nucleic acid sequence, a peptide, a lipid, a carbohydrate or a combination thereof.

31. The method of claim 29, wherein said delivery vehicle is selected from the group consisting of a viral vector or a non-viral vector.

32. The method of claim 25, wherein said cell contains an alteration in an atonal-associated nucleic acid sequence or amino acid sequence.

33. The method of claim 32, wherein said amino acid sequence has at least about 80% identity to about 20 contiguous amino acid residues of SEQ ID NO:58 (Hath1).

34. The method of claim 32, wherein said nucleic acid sequence encodes a polypeptide which has at least about 80% identity to about 20 contiguous amino acid residues of SEQ ID NO:58 (Hath1).

35. A composition in a pharmaceutical carrier, comprising:
at least one stem cell, wherein the cell is upregulated for expression of an atonal-associated sequence; and
at least one regulatory factor.

36. The composition of claim 35, wherein the stem cell is a gastrointestinal stem cell.

37. A composition in a pharmaceutical carrier, comprising:
at least one stem cell, wherein the cell is downregulated for expression of an atonal-associated sequence; and
at least one regulatory factor.

38. The composition of claim 37, wherein the stem cell is a gastrointestinal stem cell.

39. A method of treating an individual for a gastrointestinal condition, comprising the step of administering to said individual a composition of claim 36 or claim 38.

40. A method for screening for a compound in an animal, wherein said compound affects a detectable gastrointestinal condition in said animal, comprising:
delivering said compound to said animal wherein at least one allele of an atonal-associated nucleic acid sequence in said animal is inactivated by insertion of a heterologous nucleic acid sequence, wherein said heterologous nucleic acid sequence is under the control of an atonal-associated regulatory sequence, and
monitoring said animal for a change in the detectable gastrointestinal condition.

41. The method of claim 40, wherein said delivery of said compound affects expression of said heterologous nucleic acid sequence.

42. The method of claim 40 wherein said compound affects said detectable condition.

43. A kit comprising an intestinal stem cell.

44. The kit of claim 43, further comprising a regulatory protein.

45. A method of treating an animal for a disease that is a result of loss of functional atonal-associated nucleic acid or amino acid sequence comprising delivering a therapeutically effective amount of an atonal-associated amino acid sequence or nucleic acid sequence to a cell of said animal.

46. The method of claim 45, wherein said disease is a gastrointestinal disease.
